(19) United States
(12) Patent Application Publication
(10) Pub. No.: US 2024/0392000 A1
(43) Pub. Date: Nov. 28, 2024

(54) METHODS OF TREATING CANCERS ASSOCIATED WITH IMMUNOSUPPRESSIVE B CELLS

(71) Applicant: Biograph 55, Inc., San Francisco, CA (US)

(72) Inventors: Leonard PRESTA, San Francisco, CA (US); Paul TUMEH, San Francisco, CA (US); Nils LONBERG, Woodside, CA (US); Omar DURAMAD, Berkeley, CA (US)

(21) Appl. No.: 18/685,489

(22) PCT Filed: Aug. 24, 2022
(86) PCT No.: PCT/US2022/041395

§ 371 Date: Feb. 21, 2024

Related U.S. Application Data
(60) Provisional application No. 63/236,953, filed on Aug. 25, 2021.

(51) Int. Cl. C07K 16/28; A61K 39/00; A61P 35/00

(52) U.S. Cl. CPC C07K 16/2803; A61P 35/00; C07K 16/2896; A61K 2039/505; C07K 2317/31; C07K 2317/732; C07K 2317/734; C07K 2317/92

(57) ABSTRACT

Described herein is a method of treating a cancer or tumor associated with CD19 positive, CD38 positive, CD20 negative immunosuppressive B cells in an individual comprising administering to the individual a bispecific antibody that binds CD19 and CD38, thereby treating the cancer or tumor associated with CD19 positive, CD38 positive, CD20 negative immunosuppressive B cells.

### CROSS REFERENCE TO RELATED APPLICATIONS

This application claims the benefit of U.S. Provisional Ser. No. 63/236,953 filed on Aug. 25, 2021, which is incorporated by reference herein in its entirety.

### BACKGROUND

Antibody therapeutics have been used successfully to treat a variety of diseases; however, their application can be limited with respect to clinical efficacy in complex diseases such as cancer. Engineering antibody-based therapeutics to alter target-binding affinities and valences provides a potential pathway towards achieving increased efficacy and improving treatment outcomes. Bispecific or multivalent antibodies thus offer a potential approach to resolving challenges tied to the multifactorial nature of complex diseases. By binding two different antigenic molecules or different epitopes of the same antigen, bispecific antibodies offer greater functionality and offer a wide variety of applications as targeting agents for the treatment of a number of diseases.

### SUMMARY

The dynamic relationship between cancer biology and the immune system is a factor associated with clinical outcomes. The immune response plays a significant role in regulating the tumor microenvironment during cancer development. Immune cells such as T cells and B cells thus act as modulators and effectors of cancer progression or metastasis. Notably, immunosuppressive cells play an important role in the anti-tumor immune response wherein immunosuppression is generally associated with tumor growth and invasion, and correlates with negative outcomes. Although B cells are known to positively modulate the immune response, populations of immunosuppressive B cells function to suppress the anti-tumor immune response thus facilitating tumor growth.

Described herein are methods of treating certain cancers associated with immunosuppressive B-cells. The immunosuppressive B cells according to the methods are B lineage cells, B cells or plasma cells, that are CD38 positive, CD19 positive, CD20 negative. These cells show high expression of CD38 (CD38^{high}) and may be CD20^{low} or CD20^{negative}. These methods comprise administering a bispecific antibody that targets both CD19 and CD38. These method further comprise selecting a patient for such administration based on the presence of CD38high B cells or plasma cells in the patient's circulating lymphocytes or in tumor infiltrating lymphocytes. Such a targeting allows for deletion or inhibition of the function of the immunosuppressive B cells in or around the tumor or in the periphery. The deletion and/or inhibition of function of these B cells removes immunosuppression from the tumor environment and allows for an increased immune response to the tumor, including but not limited to an adaptive CD4 or CD8 T cell response.

Provided herein are certain binding molecules that target immunosuppressive B-cell populations with bispecific or multivalent targeting molecules. Targeting immune suppressive B-cell populations presents a pathway for therapeutic intervention in cancer that effectively modulates the anti-tumor immune response to improve treatment outcomes (e.g. in contrast to selective depletion of an epithelial cancer cell population). The binding molecules provided herein can comprise a bispecific antibody that binds to a B-cell lineage surface marker (e.g., CD19, CD138, IgA, and/or CD20) and a surface marker of immunosuppressive B cells (e.g., IgD, CD1, CD5, CD21, CD24, CD38, HM13, SLAMF7, AQP3, and/or latent TGF-beta (e.g., TGF-beta LAP)). In a certain specific embodiment, the bispecific antibody binds to CD19 and CD38, thus possessing selectivity for a specific immunosuppressive B-cell population.

In certain instances, the bispecific or multivalent targeting molecules target immunosuppressive B-cell populations (e.g., thereby reducing immunosuppression) to promote tumor clearance or inhibit tumor growth, as compared to targeting tumor cells directly. In such instances, when not targeting tumor cells directly, antibody-induced cell death or cell toxicity of target cells is undesirable and, furthermore, antibody-induced cell death or cell toxicity of target cells (e.g., that are not tumor cells) can lead to unwanted side effects (e.g., lymphopenia).

Described herein in a particular aspect is a method of treating a cancer or tumor associated with CD19 positive, CD38 positive, CD20 negative immunosuppressive B cells in an individual comprising administering to the individual a bispecific antibody that binds CD19 and CD38, thereby treating the cancer or tumor associated with CD19 positive, CD38 positive, CD20 negative immunosuppressive B cells. In certain embodiments, the bispecific antibody comprises a variant Fc region comprising one or more mutations relative to a wildtype Fc region, wherein the variant Fc region exhibits reduced effector function compared to the wildtype Fc region. In certain embodiments, the reduced effector function is selected from the list consisting of reduced antibody-dependent cell-mediated cytotoxicity (ADCC), reduced complement mediated cytotoxicity (CDC), reduced affinity for C1q, and any combination thereof. In certain embodiments, the variant Fc region comprises IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ec) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (oo) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (111) A330L, (mmm) P331A or P331S, or (nnn) E233P, (000) L234A, L235E, G237A, A330S, and P331S or (ppp) any combination of (a)-(uu), per Kabat numbering. In certain embodiments, the variant Fc region is selected from Table 1. In certain embodiments, the one or more mutations relative to a wildtype Fc region comprises L234A, L235E, G237A, A330S, and/or P331S by Kabat Numbering. In certain embodiments, the one or more mutations relative to a wildtype Fc region comprises L234A, L235E, G237A, A330S, and P331S by Kabat Numbering. In certain embodiments, the one or more mutations relative to a wildtype Fc region comprises K322A by Kabat Numbering. In certain embodiments, the one or more mutations relative to a wildtype Fc region consists of K322A by Kabat Numbering. In certain embodiments, the bispecific antibody that binds CD19 and CD38 comprises a CD38 antigen binding component that comprises: a) a heavy chain complementarity determining region 1 (HCDR1) comprising an amino acid sequence set forth in any one of SEQ ID NOs: 71-75; b) a heavy chain complementarity determining region 2 (HCDR2) comprising an amino acid sequence set forth in any one of SEQ ID NOs: 81-85, or 151-155; c) a heavy chain complementarity determining region 3 (HCDR3) comprising an amino acid sequence set forth in any one of SEQ ID NOs: 91-95; d) a light chain complementarity determining region 1 (LCDR1) comprising an amino acid sequence set forth in any one of SEQ ID NOs: 101-105; e) a light chain complementarity determining region 2 (LCDR2) comprising an amino acid sequence set forth in any one of SEQ ID NOs: 111-115; and f) a light chain complementarity determining region 3 (LCDR3) comprising an amino acid sequence set forth in any one of SEQ ID NOs: 121-125; and a CD19 antigen binding component that comprises: g) a heavy chain complementarity determining region 1 (HCDR1) comprising an amino acid sequence set forth in any one of SEQ ID NOs: 11-15; h) a heavy chain complementarity determining region 2 (HCDR2) comprising an amino acid sequence set forth in any one of SEQ ID NOs: 21-25; i) a heavy chain complementarity determining region 3 (HCDR3) comprising an amino acid sequence set forth in any one of SEQ ID NOs: 31-35; j) a light chain complementarity determining region 1 (LCDR1) comprising an amino acid sequence set forth in any one of SEQ ID NOs: 101-105; k) a light chain complementarity determining region 2 (LCDR2) comprising an amino acid sequence set forth in any one of SEQ ID NOs: 111-115; and 1) a light chain complementarity determining region 3 (LCDR3) comprising an amino acid sequence set forth in any one of SEQ ID NOs: 121-125. In certain embodiments, the CD38 antigen binding component comprises an HCDR2 amino acid sequence comprising the amino acid sequence set forth in SEQ ID NO: 151 to 155. In certain embodiments, the CD38 antigen binding component comprises an HCDR2 amino acid sequence comprising the amino acid sequence set forth in SEQ ID NO: 154. In certain embodiments, the bispecific antibody that binds CD19 and CD38 comprises a CD38 antigen binding component comprises an HCDR2 amino acid sequence comprising any one of the amino acid sequences set forth in SEQ ID NO: 81 to 85. In certain embodiments, the bispecific antibody that binds CD19 and CD38 comprises an anti-CD38 immunoglobulin heavy chain variable region that comprises an amino acid sequence having at least about 90% identity to SEQ ID NO: 3 or 5; and the anti-CD38 immunoglobulin light chain variable region comprises an amino acid sequence having at least about 90% identity to SEQ ID NO: 4. In certain embodiments, the bispecific antibody that binds CD19 and CD38 comprises a anti-CD38 immunoglobulin heavy chain variable region that comprises an amino acid sequence identical to SEQ ID NO: 3 or 5; and the anti-CD38 immunoglobulin light chain variable region comprises an amino acid sequence identical to SEQ ID NO: 4. In certain embodiments, the bispecific antibody that binds CD19 and CD38 comprises an anti-CD19 immunoglobulin heavy chain variable region that comprises an amino acid sequence having at least about 90% identity to SEQ ID NO: 1, 6 or 7; and the anti-CD19 immunoglobulin light chain variable region comprises an amino acid sequence having at least about 90% identity to SEQ ID NO: 2. In certain embodiments, the bispecific antibody that binds CD19 and CD38 comprises an anti-CD19 immunoglobulin heavy chain variable region that comprises an amino acid sequence identical to SEQ ID NO: 1, 6 or 7; and the anti-CD19 immunoglobulin light chain variable region comprises an amino acid sequence identical to SEQ ID NO: 2. In certain embodiments, the bispecific antibody that binds CD19 and CD38 comprises a anti-CD38 immunoglobulin heavy chain variable region further comprises an immunoglobulin heavy chain constant region, wherein the anti-CD38 immunoglobulin heavy chain constant region comprises one or more amino acid substitutions that disfavors homodimerization of the anti-CD38 immunoglobulin heavy chain constant region and promotes heterodimerization of the anti-CD38 immunoglobulin heavy chain constant region with a non-anti-CD38 immunoglobulin heavy chain constant region. In certain embodiments, the bispecific antibody that binds CD19 and CD38 comprises an anti-CD38 immunoglobulin heavy chain constant region comprises a T366W substitution (EU numbering) or T366S/L368A/Y407V substitution (EU numbering), such that the heterodimerization of the anti-CD38 immunoglobulin heavy chain constant region and the non-anti-CD38 immunoglobulin heavy chain constant region is favored compared to homodimerization of the anti-CD38 immunoglobulin heavy chain. In certain embodiments, the bispecific antibody comprises an anti-CD19 immunoglobulin heavy chain variable region further comprises an immunoglobulin heavy chain constant region, wherein the anti-CD19 immunoglobulin heavy chain constant region comprises one or more amino acid substitutions that disfavors homodimerization of the anti-CD19 immunoglobulin heavy chain constant region and promotes heterodimerization of the second heavy chain constant region with a non-anti-CD19 immunoglobulin heavy chain constant region. In certain embodiments, the anti-CD19 immunoglobulin heavy chain constant region comprises a T366W substitution (EU numbering) or a T366S/L368A/Y407V substitution (EU numbering), such that the heterodimerization of the anti-CD19 immunoglobulin heavy chain constant region and the non-anti-CD19 immunoglobulin heavy chain immunoglobulin heavy chain constant region is favored compared to homodimerization of the anti-CD19 immunoglobulin heavy chain. In certain embodiments, the anti-CD38 immunoglobulin light chain variable region further comprises an immunoglobulin light chain constant region. In certain embodiments, the bispecific antibody that binds CD19 and CD38 comprises a CD19 antigen binding component comprises a heavy chain immunoglobulin sequence set forth in SEQ ID NO: 301 or 304 and a light chain immunoglobulin sequence set forth in SEQ ID NO: 213, and the CD38 binding component comprises a heavy chain immunoglobulin sequence set forth in SEQ ID NO: 302, 303, 305-310 and a light chain immunoglobulin sequence set forth in SEQ ID NO: 213. In certain embodiments, the bispecific antibody that binds CD19 and CD38 comprises an anti-CD19 immunoglobulin heavy chain variable region that comprises an A84S or an A108L substitution according to Kabat numbering In certain embodiments, the bispecific antibody that binds CD19 and CD38 comprises an anti-CD38 immunoglobulin light chain variable region comprises a W32H substitution according to Kabat numbering. In certain embodiments, a single bispecific binding molecule is formed from the CD38 antigen binding component and the CD19 antigen binding component. In certain embodiments, the composite binding molecules is a common light chain bispecific antibody. In certain embodiments, the bispecific antibody that binds CD19 and CD38 is included in a formulation comprising a pharmaceutically acceptable diluent, carrier, or excipient. In certain embodiments, the cancer or tumor is a solid-tissue cancer. In certain embodiments, the solid-tissue cancer comprises breast cancer, prostate cancer, pancreatic cancer, lung cancer, kidney cancer, stomach cancer, esophageal cancer, skin cancer, colorectal cancer, or head and neck cancer. In certain embodiments, the breast cancer is triple negative breast cancer, the lung cancer is non-small cell lung cancer, the head and neck cancer is head and neck squamous cell cancer, the kidney cancer is renal cell carcinoma, the brain cancer is glioblastoma multiforme, or the skin cancer is melanoma. In certain embodiments, the cancer or tumor associated with CD19 positive, CD38 positive, CD20 negative immunosuppressive B cells is a cancer or tumor that comprises CD19 positive, CD38 positive, CD20 negative B cell infiltrates. In certain embodiments, the CD19 positive, CD38 positive, CD20 negative immunosuppressive B cells express a B cell activation marker. In certain embodiments, the B cell activation marker comprises CD30.

Described herein is a method of treating an individual afflicted with a tumor or cancer, the method comprising performing an assay on the B cells of a biological sample of the individual for a CD38 high phenotype; and administering a bispecific antibody that binds CD19 and CD38 to the individual afflicted with the tumor or the cancer based on results of the assay on the B cells of a biological sample from the individual. In certain embodiments, the results of the assay of the B cells of the biological sample of the individual indicate a CD38 high phenotype. In certain embodiments, the biological sample of the individual is a peripheral blood sample. In certain embodiments, the biological sample of the individual is a tumor biopsy. In certain embodiments, the assay the B cells of the individual comprises contacting the biological sample with an anti-CD38 antibody. In certain embodiments, the assay comprises flow cytometry. In certain embodiments, the assay comprises immunohistochemistry. In certain embodiments, the individual is administered a bispecific antibody that binds CD38 and CD19 to the individual afflicted with the tumor or the cancer if greater than about 2% of the B cells of the individual exhibit a CD38 high phenotype. In certain embodiments, the B cells of the biological sample of the individual indicate a CD38 high phenotype if the B cells express greater than about 30,000 cell surface CD38 molecules. In certain embodiments, the B cells of the biological sample of the individual indicate a CD38 high phenotype if the B cells express greater than about 35,000 cell surface CD38 molecules. In certain embodiments, the B cells of the biological sample of the individual indicate a CD38 high phenotype if the B cells express greater than about 40,000 cell surface CD38 molecules. In certain embodiments, the bispecific antibody that binds CD19 and CD38 comprises a CD38 antigen binding component that comprises: a) a heavy chain complementarity determining region 1 (HCDR1) comprising an amino acid sequence set forth in any one of SEQ ID NOs: 71-75; b) a heavy chain complementarity determining region 2 (HCDR2) comprising an amino acid sequence set forth in any one of SEQ ID NOs: 81-85, or 151-155; c) a heavy chain complementarity determining region 3 (HCDR3) comprising an amino acid sequence set forth in any one of SEQ ID NOs: 91-95; d) a light chain complementarity determining region 1 (LCDR1) comprising an amino acid sequence set forth in any one of SEQ ID NOs: 101-105; e) a light chain complementarity determining region 2 (LCDR2) comprising an amino acid sequence set forth in any one of SEQ ID NOs: 111-115; and f) a light chain complementarity determining region 3 (LCDR3) comprising an amino acid sequence set forth in any one of SEQ ID NOs: 121-125; and wherein a CD19 antigen binding component comprises: g) a heavy chain complementarity determining region 1 (HCDR1) comprising an amino acid sequence set forth in any one of SEQ ID NOs: 11-15; h) a heavy chain complementarity determining region 2 (HCDR2) comprising an amino acid sequence set forth in any one of SEQ ID NOs: 21-25; i) a heavy chain complementarity determining region 3 (HCDR3) comprising an amino acid sequence set forth in any one of SEQ ID NOs: 31-35; j) a light chain complementarity determining region 1 (LCDR1) comprising an amino acid sequence set forth in any one of SEQ ID NOs: 101-105; k) a light chain complementarity determining region 2 (LCDR2) comprising an amino acid sequence set forth in any one of SEQ ID NOS: 111-115; and 1) a light chain complementarity determining region 3 (LCDR3) comprising an amino acid sequence set forth in any one of SEQ ID NOs: 121-125. In certain embodiments, the CD38 antigen binding component comprises an HCDR2 amino acid sequence comprising the amino acid sequence set forth in SEQ ID NO: 151 to 155. In certain embodiments, the CD38 antigen binding component comprises an HCDR2 amino acid sequence comprising the amino acid sequence set forth in SEQ ID NO: 154. In certain embodiments, the bispecific antibody that binds CD19 and CD38 comprises a CD38 antigen binding component comprises an HCDR2 amino acid sequence comprising any one of the amino acid sequences set forth in SEQ ID NO: 81 to 85. In certain embodiments, the bispecific antibody that binds CD19 and CD38 comprises an anti-CD38 immunoglobulin heavy chain variable region that comprises an amino acid sequence having at least about 90% identity to SEQ ID NO: 3 or 5; and the anti-CD38 immunoglobulin light chain variable region comprises an amino acid sequence having at least about 90% identity to SEQ ID NO: 4. In certain embodiments, the bispecific antibody that binds CD19 and CD38 comprises a anti-CD38 immunoglobulin heavy chain variable region that comprises an amino acid sequence identical to SEQ ID NO: 3 or 5; and the anti-CD38 immunoglobulin light chain variable region comprises an amino acid sequence identical to SEQ ID NO: 4. In certain embodiments, the bispecific antibody that binds CD19 and CD38 comprises an anti-CD19 immunoglobulin heavy chain variable region that comprises an amino acid sequence having at least about 90% identity to SEQ ID NO: 1 or 6; and the anti-CD19 immunoglobulin light chain variable region comprises an amino acid sequence having at least about 90% identity to SEQ ID NO: 4. In certain embodiments, the bispecific antibody that binds CD19 and CD38 comprises an anti-CD19 immunoglobulin heavy chain variable region that comprises an amino acid sequence identical to SEQ ID NO: 1 or 6; and the anti-CD19 immunoglobulin light chain variable region comprises an amino acid sequence identical to SEQ ID NO: 4. In certain embodiments, the bispecific antibody that binds CD19 and CD38 comprises a anti-CD38 immunoglobulin heavy chain variable region further comprises an immunoglobulin heavy chain constant region, wherein the anti-CD38 immunoglobulin heavy chain constant region comprises one or more amino acid substitutions that disfavors homodimerization of the anti-CD38 immunoglobulin heavy chain constant region and promotes heterodimerization of the anti-CD38 immunoglobulin heavy chain constant region with a non-anti-CD38 immunoglobulin heavy chain constant region. In certain embodiments, the bispecific antibody that binds CD19 and CD38 comprises an anti-CD38 immunoglobulin heavy chain constant region comprises a T366W substitution (EU numbering) or T366S/L368A/Y407V substitution (EU numbering), such that the heterodimerization of the anti-CD38 immunoglobulin heavy chain constant region and the non-anti-CD38 immunoglobulin heavy chain constant region is favored compared to homodimerization of the anti-CD38 immunoglobulin heavy chain. In certain embodiments, the bispecific antibody comprises an anti-CD19 immunoglobulin heavy chain variable region further comprises an immunoglobulin heavy chain constant region, wherein the anti-CD19 immunoglobulin heavy chain constant region comprises one or more amino acid substitutions that disfavors homodimerization of the anti-CD19 immunoglobulin heavy chain constant region and promotes heterodimerization of the second heavy chain constant region with a non-anti-CD19 immunoglobulin heavy chain constant region. In certain embodiments, the anti-CD19 immunoglobulin heavy chain constant region comprises a T366W substitution (EU numbering) or a T366S/L368A/Y407V substitution (EU numbering), such that the heterodimerization of the anti-CD19 immunoglobulin heavy chain constant region and the non-anti-CD19 immunoglobulin heavy chain immunoglobulin heavy chain constant region is favored compared to homodimerization of the anti-CD19 immunoglobulin heavy chain. In certain embodiments, the anti-CD38 immunoglobulin light chain variable region further comprises an immunoglobulin light chain constant region. In certain embodiments, the bispecific antibody that binds CD19 and CD38 comprises a CD19 antigen binding component comprises a heavy chain immunoglobulin sequence set forth in SEQ ID NO: 301 or 304 and a light chain immunoglobulin sequence set forth in SEQ ID NO: 213, and the CD38 binding component comprises a heavy chain immunoglobulin sequence set forth in SEQ ID NO: 302, 303, 305-310 and a light chain immunoglobulin sequence set forth in SEQ ID NO: 213. In certain embodiments, the bispecific antibody that binds CD19 and CD38 comprises an anti-CD19 immunoglobulin heavy chain variable region that comprises an A84S or an A108L substitution according to Kabat numbering In certain embodiments, the bispecific antibody that binds CD19 and CD38 comprises an anti-CD38 immunoglobulin light chain variable region comprises a W32H substitution according to Kabat numbering. In certain embodiments, a single bispecific binding molecule is formed from the CD38 antigen binding component and the CD19 antigen binding component. In certain embodiments, the bispecific antibody that binds CD19 and CD38 is a common light chain bispecific antibody. In certain embodiments, the bispecific antibody that binds CD19 and CD38 is included in a formulation comprising a pharmaceutically acceptable diluent, carrier, or excipient.

Also described herein is a method of treating an individual afflicted with a tumor or cancer, the method comprising administering a bispecific antibody that binds CD19 and CD38 to the individual afflicted with the tumor or the cancer based on results of an assay on B cells of a biological sample of the individual. In certain embodiments, the results of the assay of the B cells of the biological sample of the individual indicate a CD38 high phenotype. In certain embodiments, the biological sample of the individual is a peripheral blood sample. In certain embodiments, the biological sample of the individual is a tumor biopsy. In certain embodiments, the assay the B cells of the individual comprises contacting the biological sample with an anti-CD38 antibody. In certain embodiments, the assay comprises flow cytometry. In certain embodiments, the assay comprises immunohistochemistry. In certain embodiments, the individual is administered a bispecific antibody that binds CD38 and CD19 to the individual afflicted with the tumor or the cancer if greater than about 2% of the B cells of the individual exhibit a CD38 high phenotype. In certain embodiments, the B cells of the biological sample of the individual indicate a CD38 high phenotype if the B cells express greater than about 30,000 cell surface CD38 molecules. In certain embodiments, the B cells of the biological sample of the individual indicate a CD38 high phenotype if the B cells express greater than about 35,000 cell surface CD38 molecules. In certain embodiments, the B cells of the biological sample of the individual indicate a CD38 high phenotype if the B cells express greater than about 40,000 cell surface CD38 molecules. In certain embodiments, the bispecific antibody that binds CD19 and CD38 comprises a CD38 antigen binding component that comprises: a) a heavy chain complementarity determining region 1 (HCDR1) comprising an amino acid sequence set forth in any one of SEQ ID NOs: 71-75; b) a heavy chain complementarity determining region 2 (HCDR2) comprising an amino acid sequence set forth in any one of SEQ ID NOs: 81-85, or 151-155; c) a heavy chain complementarity determining region 3 (HCDR3) comprising an amino acid sequence set forth in any one of SEQ ID NOs: 91-95; d) a light chain complementarity determining region 1 (LCDR1) comprising an amino acid sequence set forth in any one of SEQ ID NOs: 101-105; e) a light chain complementarity determining region 2 (LCDR2) comprising an amino acid sequence set forth in any one of SEQ ID NOs: 111-115; and f) a light chain complementarity determining region 3 (LCDR3) comprising an amino acid sequence set forth in any one of SEQ ID NOs: 121-125; and wherein a CD19 antigen binding component comprises: g) a heavy chain complementarity determining region 1 (HCDR1) comprising an amino acid sequence set forth in any one of SEQ ID NOs: 11-15; h) a heavy chain complementarity determining region 2 (HCDR2) comprising an amino acid sequence set forth in any one of SEQ ID NOs: 21-25; i) a heavy chain complementarity determining region 3 (HCDR3) comprising an amino acid sequence set forth in any one of SEQ ID NOs: 31-35; j) a light chain complementarity determining region 1 (LCDR1) comprising an amino acid sequence set forth in any one of SEQ ID NOs: 101-105; k) a light chain complementarity determining region 2 (LCDR2) comprising an amino acid sequence set forth in any one of SEQ ID NOs: 111-115; and 1) a light chain complementarity determining region 3 (LCDR3) comprising an amino acid sequence set forth in any one of SEQ ID NOs: 121-125. In certain embodiments, the CD38 antigen binding component comprises an HCDR2 amino acid sequence comprising the amino acid sequence set forth in SEQ ID NO: 151 to 155. In certain embodiments, the CD38 antigen binding component comprises an HCDR2 amino acid sequence comprising the amino acid sequence set forth in SEQ ID NO: 154. In certain embodiments, the bispecific antibody that binds CD19 and CD38 comprises a CD38 antigen binding component comprises an HCDR2 amino acid sequence comprising any one of the amino acid sequences set forth in SEQ ID NO: 81 to 85. In certain embodiments, the bispecific antibody that binds CD19 and CD38 comprises an anti-CD38 immunoglobulin heavy chain variable region that comprises an amino acid sequence having at least about 90% identity to SEQ ID NO: 3 or 5; and the anti-CD38 immunoglobulin light chain variable region comprises an amino acid sequence having at least about 90% identity to SEQ ID NO: 4. In certain embodiments, the bispecific antibody that binds CD19 and CD38 comprises a anti-CD38 immunoglobulin heavy chain variable region that comprises an amino acid sequence identical to SEQ ID NO: 3 or 5; and the anti-CD38 immunoglobulin light chain variable region comprises an amino acid sequence identical to SEQ ID NO: 4. In certain embodiments, the bispecific antibody that binds CD19 and CD38 comprises an anti-CD19 immunoglobulin heavy chain variable region that comprises an amino acid sequence having at least about 90% identity to SEQ ID NO: 1 or 6; and the anti-CD19 immunoglobulin light chain variable region comprises an amino acid sequence having at least about 90% identity to SEQ ID NO: 4. In certain embodiments, the bispecific antibody that binds CD19 and CD38 comprises an anti-CD19 immunoglobulin heavy chain variable region that comprises an amino acid sequence identical to SEQ ID NO: 1 or 6; and the anti-CD19 immunoglobulin light chain variable region comprises an amino acid sequence identical to SEQ ID NO: 4. In certain embodiments, the bispecific antibody that binds CD19 and CD38 comprises a anti-CD38 immunoglobulin heavy chain variable region further comprises an immunoglobulin heavy chain constant region, wherein the anti-CD38 immunoglobulin heavy chain constant region comprises one or more amino acid substitutions that disfavors homodimerization of the anti-CD38 immunoglobulin heavy chain constant region and promotes heterodimerization of the anti-CD38 immunoglobulin heavy chain constant region with a non-anti-CD38 immunoglobulin heavy chain constant region. In certain embodiments, the bispecific antibody that binds CD19 and CD38 comprises an anti-CD38 immunoglobulin heavy chain constant region comprises a T366W substitution (EU numbering) or T366S/L368A/Y407V substitution (EU numbering), such that the heterodimerization of the anti-CD38 immunoglobulin heavy chain constant region and the non-anti-CD38 immunoglobulin heavy chain constant region is favored compared to homodimerization of the anti-CD38 immunoglobulin heavy chain. In certain embodiments, the bispecific antibody comprises an anti-CD19 immunoglobulin heavy chain variable region further comprises an immunoglobulin heavy chain constant region, wherein the anti-CD19 immunoglobulin heavy chain constant region comprises one or more amino acid substitutions that disfavors homodimerization of the anti-CD19 immunoglobulin heavy chain constant region and promotes heterodimerization of the second heavy chain constant region with a non-anti-CD19 immunoglobulin heavy chain constant region. In certain embodiments, the anti-CD19 immunoglobulin heavy chain constant region comprises a T366W substitution (EU numbering) or a T366S/L368A/Y407V substitution (EU numbering), such that the heterodimerization of the anti-CD19 immunoglobulin heavy chain constant region and the non-anti-CD19 immunoglobulin heavy chain immunoglobulin heavy chain constant region is favored compared to homodimerization of the anti-CD19 immunoglobulin heavy chain. In certain embodiments, the anti-CD38 immunoglobulin light chain variable region further comprises an immunoglobulin light chain constant region. In certain embodiments, the bispecific antibody that binds CD19 and CD38 comprises a CD19 antigen binding component comprises a heavy chain immunoglobulin sequence set forth in SEQ ID NO: 301 or 304 and a light chain immunoglobulin sequence set forth in SEQ ID NO: 213, and the CD38 binding component comprises a heavy chain immunoglobulin sequence set forth in SEQ ID NO: 302, 303, 305-310 and a light chain immunoglobulin sequence set forth in SEQ ID NO: 213. In certain embodiments, the bispecific antibody that binds CD19 and CD38 comprises an anti-CD19 immunoglobulin heavy chain variable region that comprises an A84S or an A108L substitution according to Kabat numbering. In certain embodiments, the bispecific antibody that binds CD19 and CD38 comprises an anti-CD38 immunoglobulin light chain variable region comprises a W32H substitution according to Kabat numbering. In certain embodiments, a single bispecific binding molecule is formed from the CD38 antigen binding component and the CD19 antigen binding component. In certain embodiments, the bispecific antibody that binds CD19 and CD38 is a common light chain bispecific antibody. In certain embodiments, the bispecific antibody that binds CD19 and CD38 is included in a formulation comprising a pharmaceutically acceptable diluent, carrier, or excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the structure of a common light chain bispecific IgG.

FIG. 2 illustrates the structure of a Fab-Fc:scFv-Fc bispecific IgG.

FIG. 3 illustrates the structure of a Fab-Fc-Fab:Fc bispecific IgG.

FIG. 4 illustrates the structure of a Fab-Fc-scFv:Fab-Fc-scFv bispecific IgG.

FIG. 5 illustrates the structure of a Fab-Fc-scFv:Fc bispecific IgG.

FIG. 6 illustrates the structure of a Fab-Fc-Fab:Fab-Fc bispecific IgG.

FIG. 7 illustrates the structure of an scFv-Fab-Fc:scFv-Fab-Fc bispecific IgG.

FIG. 8 illustrates the structure of a Fab-Fab-Fc:Fab-Fab-Fc bispecific IgG.

FIG. 9 illustrates the structure of a Fab-Fc-Fab:Fab-Fc-Fab bispecific IgG.

FIG. 10 illustrates the structure of a Fab-Fc-scFv:Fab-Fc bispecific IgG.

FIG. 11 illustrates the structure of an scFv-Fab-Fc:Fc Bispecific IgG.

FIG. 12A to 12B shows binding data of antibodies to Daudi cells.

FIG. 13A to 13B shows binding data of antibodies to REH cells.

FIG. 14A to 14B shows binding data of antibodies to CD19 transfected HEK293 cells.

FIG. 15A to 15B shows binding data of antibodies to CD38 transfected HEK293 cells.

FIG. 16A to 16B shows binding data of antibodies to non-transfected CHO cells.

FIG. 17A to 17B shows data for direct apoptosis on Daudi cells for antibody test articles.

FIG. 18A to 18B shows data for cross-linking induced apoptosis on Daudi cells for antibody test articles.

FIG. 19A to 19C shows ADCC data for three donors across antibody test articles.

FIG. 20A to 20C shows ADCC data for three donors across antibody test articles.

FIG. 21A to 21B shows CDC profiles across test articles.

FIG. 22 shows ADCP data across antibody test articles.

FIG. 23 shows RBC binding data across antibody test articles.

FIG. 24A to 24B shows hemagglutination profiles for antibody test articles.

FIG. 25 shows hemolysis data across antibody test articles.

FIG. 26A to 26G shows ADCC data for three donors across antibody test articles, including those with a variant.

FIG. 27A shows flow cytometry analysis of CD20−, CD19+, CD38+ cell compartment in peripheral blood of healthy donors and a non-small cell lung cancer patient.

FIG. 27B shows flow cytometry analysis of CD20−, CD19+, CD38+ cell compartment in peripheral blood of patients with specific tumor-types, with the exception of HCC which is from tumors.

FIG. 28 shows receptor density of CD19 and CD38 on CD20-cells from tumors and peripheral blood of patients with different types of cancer.

FIG. 29 shows a positive correlation between peripheral blood and tumor for CD38 receptor levels in CD20−, CD19+, CD38+ patients.

FIG. 30 shows that CD19 and CD38+ cells in tumors and peripheral blood of cancer patients secrete the immunosuppressive cytokine IL-10.

### DETAILED DESCRIPTION

Immunosuppressive B-cell populations (i.e., regulatory B cells or Bregs) that suppress the anti-tumor immune response can be generally defined by the presence of more than one cell surface biomarker. Therapeutics that effectively and specifically target immunosuppressive B cells can therefore be used to prevent immunosuppression and/or remove immunosuppression in, adjacent to, or surrounding a tumor or within a tumor environment. Provided herein are composite binding molecules that target immunosuppressive B cells. Furthermore, provided are composite binding molecules comprising a first binding component configured to bind a first target and a second binding component configured to bind a second target, wherein the first target comprises a B-cell lineage surface marker, and wherein the second target comprises a suppressive B-cell surface marker. Disclosed herein are multivalent antibodies that specifically bind to B-cell populations associated with negative modulation or immunosuppression of an anti-tumor response. Immunosuppressive B cells can comprise or be defined by cell surface biomarkers CD19 and CD38. The bispecific antibodies provided herein can target both CD19 and CD38 to inhibit the function of immune suppressive B cells. In certain instances, the function of immunosuppressive B cells comprises the release or expression of IL10, IL 35, TGF-beta, or a combination thereof. Multivalent or bispecific antibodies targeting CD19 and CD38 can also be used for treating tumorigenic conditions and/or cancers associated with immunosuppressive B cells and/or immune dysfunction.

The term “immunosuppression” or “immunodepression” or “negative immune modulation”, or “regulatory” in reference to particular cell populations as used herein, refers to processes or cells that are responsible for the reduction or suppression of the immune system function. Immunosuppression generally denotes a state when immune system function is reduced or absent with respect to one or functions such as cellular immunity, antibody-based immunity, or innate immune function. In certain instances, immunosuppression generally denotes a state when immune system function against a tumor or within, surrounding, or adjacent to the tumor microenvironment is reduced or absent. The whole immune response may be depressed, the immune response within a local or specific region may be reduced, or a particular population of immunologically active lymphocytes may be selectively affected. Antigen-specific immunosuppression may be the result of deletion or suppression of a particular population of antigen-specific cells, or the result of enhanced regulation of the immune response by antigen-specific suppressor cells. References to immunosuppressive B cells refer to B cells or B-cell populations that exert negative modulation on the immune response and can be identified by specific surface markers associated with such populations, such as CD38. In certain instances, immunosuppression can be identified by the presence or release of IL-10, IL-35, TGF-beta, or a combination thereof. In certain instances, immunosuppression can be identified by the presence or release by B cells of IL-10, IL-35, TGF-beta, or a combination thereof.

As used herein, the term “cancer” can refer to or describe the physiological condition in mammals that is typically characterized by unregulated cell growth. Cancer can also include, solid tumors. Cancer can refer to diseases of the blood, bones, organs, skin tissues and vascular system, including but not limited to bladder, blood, bones, brain, breast, cervix, chest, colon, endometrium, esophagus, eyes, head, kidneys, liver, lungs, lymph nodes, mouth, neck, ovaries, pancreas, prostate, rectum, kidney, skin, stomach, testes, throat and uterus. Specific cancers include, but are not limited to, gastrointestinal tumor (e.g., gastrointestinal stromal tumor (GIST)), follicular lymphoma, mantle cell lymphoma/leukemia, Diffuse B-cell lymphoma, mediastinal (thymus) large B-cell lymphoma, intravascular large B-cell lymphoma, primary exudative lymphoma, and Burkitt's lymphoma (Burkitt lymphoma), mature T cells and natural killer cell (NK) tumors (pre-lymphocytic leukemia, T-cell large lymphocytic leukemia, invasive NK cell leukemia, adult T-cell leukemia/lymphoma, Extranodal NK/T-cell lymphoma, enteropathic T-cell lymphoma, hepatosplenic T-cell lymphoma, blastic NK cell lymphoma, mycosis fungoides (Sezary syndrome), primary Skin degenerative large cell lymphoma, lymphomatoid papulosis, angioimmunoblastic T-cell lymphoma, unspecified peripheral T-cell lymphoma and degenerative large cell lymphoma, Hodgkin's lymphoma (nodular sclerosis, mixed cell type, lymphocyte rich type, lymphocyte depleted or unreduced type, nodular lymphocyte type), myeloma (multiple myeloma, inert myeloma, smoldering myeloma)), chronic myeloproliferative diseases, myelodysplasia/myeloproliferative diseases, myelodysplastic syndromes, lymphoproliferative disorders associated with immunodeficiency, histiocytic and dendritic cell tumors, Hypercytosis, chondrosarcoma, Ewing sarcoma, fibrosarcoma, malignant giant cell tumor, myeloma bone disease, osteosarcoma, breast cancer (hormone dependent, non-hormone dependent), gynecological cancer (child Cervical, endometrial, fallopian tube, gestational trophoblastic disease, ovary, peritoneum, uterus, vagina and vulva), basal cell carcinoma (BCC), squamous cell carcinoma (SCC), malignant melanoma, protuberous cutaneous fibrosarcoma, Merkel cell carcinoma, Kaposi's sarcoma, astrocytoma, hair cell astrocytoma, embryonic hair growth neuroepithelial neoplasia, oligodendroglioma, Ependymoma, glioblastoma multiforme, mixed glioma, oligodendrocyte astrocytoma, medulloblastoma, retinoblastoma, neuroblastoma, embryonal tissue tumor, teratoma, Malignant mesothelioma (peritoneal mesothelioma, pericardial mesothelioma, pleural mesothelioma), gastric-intestinal-pancreatic or gastrointestinal pancreatic neuroendocrine tumor (GEP-NET), carcinoid tumor, pancreatic endocrine tumor (PET)), colorectal adenocarcinoma, knot Rectal cancer, invasive neuroendocrine tumor, leiomyosarcoma, mucinous adenocarcinoma, signet ring cell adenocarcinoma, hepatocellular carcinoma, hepatobiliary liver cancer, hepatic blastoma, hemangioma, hepatic adenoma, focal nodular hyperplasia (nodular regenerative hyperplasia, hamartoma), non-small cell lung cancer (NSCLC) (squamous cell lung cancer, adenocarcinoma, large cell lung cancer), small cell lung cancer, thyroid cancer, prostate cancer (hormone refractory, non-androgen dependent Sex, androgen-dependent, hormone-insensitive), renal cell carcinoma and soft tissue sarcoma (fibrosarcoma, malignant fibrous histiocytoma, cutaneous fibrosarcoma, liposarcoma, rhabdomyosarcoma, leiomyosarcoma, angiosarcoma, synovial sarcoma, malignant Peripheral nerve sheath tumor/neurofibrosarcoma, extra-osseous osteosarcoma).

The term “CD19” or “Cluster of Differentiation 19” (also known as B4, T-cell surface antigen Leu-12, and CVID3) refers to a B-cell lineage surface biomarker or transmembrane protein that in humans is encoded by the gene CD19. CD19 can function as coreceptor for the B-cell antigen receptor complex (BCR) on B-lymphocytes, which decreases the threshold for activation of downstream signaling pathways and for triggering B cell responses to antigens. Structurally, a CD19 amino acid sequence has at least about 90%, 91%, 92%, 93%, 94%, 95%, 96%, 97%, 98%, 99% or 100% sequence identity with the amino acid sequence, e.g., of GenBank accession no. NM_001178098.2→NP_001171569.1 or NM_001770.6→NP_001761.3 over a sequence length of at least 50,100,150, 200, 250, 300, 350, 400, 450, 500 amino acids or over the full length of the polypeptide. Structurally, a CD19 nucleic acid sequence has at least about 90%, 91%, 92%, 93%, 94%, 95%, 96%, 97%, 98%, 99% or 100% sequence identity with the nucleic acid sequence, e.g., of GenBank accession no. NG_007275.1 or NCBI Gene ID 930, over a sequence length of at least 300, 500, 750, 1000, 1250, 1500 nucleic acids or over the full length of the polynucleotide. The sequence alignments can be performed using any alignment algorithm known in the art, e.g., BLAST, ALIGN, set to default settings.

The term “CD38” or “Cluster of Differentiation 38” (also known as ADPRC1) refers to a B-cell surface biomarker or transmembrane protein that in humans is encoded by the gene CD38. CD38 can function in B-cell signaling that leads to cellular activation and proliferation. Structurally, a CD38 amino acid sequence has at least about 90%, 91%, 92%, 93%, 94%, 95%, 96%, 97%, 98%, 99% or 100% sequence identity with the amino acid sequence, e.g., of GenBank accession no. NM_001775.4→NP_001766.2 over a sequence length of at least 50, 100, 150, 200, 250, amino acids or over the full length of the polypeptide. There is a second isoform of CD38 with a premature stop codon that may be expressed at low levels in some cells. Structurally, an CD19 nucleic acid sequence has at least about 90%, 91%, 92%, 93%, 94%, 95%, 96%, 97%, 98%, 99% or 100% sequence identity with the nucleic acid sequence, e.g., of GenBank accession no. NC_000004.12 or NCBI Gene ID 952, over a sequence length of at least 300, 500, 750 nucleic acids or over the full length of the polynucleotide. The sequence alignments can be performed using any alignment algorithm known in the art, e.g., BLAST, ALIGN, set to default settings.

The term “CD20” or “Cluster of Differentiation 20” (also known as B-lymphocyte surface antigen B1) refers to a B-cell lineage surface biomarker or transmembrane protein that in humans is encoded by the gene CD20. Structurally, a CD20 amino acid sequence has at least about 90%, 91%, 92%, 93%, 94%, 95%, 96%, 97%, 98%, 99% or 100% sequence identity with the amino acid sequence, e.g., of Uniprot entry P11836 over a sequence length of at least 50, 100, 150, 200, 250, amino acids or over the full length of the polypeptide.

As used herein the term “biological sample” refers to any sample comprising one or more biological macromolecules (e.g., polypeptides, nucleic acids, or cells). Biological samples can be derived form individuals and include, without limitation, biopsy samples of diseased tissue (or tissue suspected of being diseased), blood, serum, or plasma samples, fecal samples, saliva samples, urine samples, lavage samples, buccol or nasopharyngeal swabs and the like. Biological samples can be subjected to further processing, including but not limited to, refrigeration, freezing, fixation, filtration, enzyme treatment, centrifugation, washing, extraction (e.g., of cells, polypeptides, or nucleic acids) and still be considered biological samples.

As described herein “assay” refers to any method or procedure used to determine the presence or absence of a specific biological macromolecule including quantitative, qualitative, or comparative amounts of the biological macromolecule (e.g., polypeptides, nucleic acids, cells, tissues, etc.).

As described herein with reference to binding molecules such as antibodies and bispecific antibodies “binding” refers the specific interaction of a target antigen with one or more amino acid residues of a variable region of complementarity determining region. Such specific biding will generally result in a dissociation constant of less than about 1×10⁻⁶ M, such affinity can be determined by the skilled artisan using techniques known in the art, such as by surface plasmon resonance.

The term “antibody” herein is used in the broadest sense and includes multivalent or bispecific antibodies and monoclonal antibodies, including intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

Among the provided antibodies are multispecific or multivalent antibodies (for example, bispecific antibodies and polyreactive antibodies) and antibody fragments thereof. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. A monoclonal antibody can comprise a human IgG1 constant region or a human IgG4 constant region.

The terms “complementarity determining region,” and “CDR,” which are synonymous with “hypervariable region” or “HVR,” are known in the art and refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 December; 13 (12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Among the provided antibodies are antibody fragments. An “antibody fragment” can refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂: diabodies; linear antibodies; single-chain antibody molecules (e.g. scFv or sFv); and multispecific antibodies formed from antibody fragments. In particular embodiments, the antibodies are single-chain antibody fragments comprising a variable heavy chain region and/or a variable light chain region, such as scFvs. Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody.

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding may be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides. For example, two distinct binding moieties or a heavy-chain/light-chain pair. A number of strategies may be used to covalently link molecules together. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length may be used. Useful linkers include glycine-serine polymers, including for example (GS)n, (GSGGS)n (SEQ ID NO: 224), (GGGGS)n (SEQ ID NO: 225), and (GGGS)n (SEQ ID NO: 226), where n is an integer of at least one, glycine-alanine polymers, alanine-serine polymers, and other flexible linkers. Exemplary, linkers for linking antibody fragments or single chain variable fragments can include AAEPKSS (SEQ ID NO: 227), AAEPKSSDKTHTCPPCP (SEQ ID NO: 228), GGGG (SEQ ID NO: 229), or GGGGDKTHTCPPCP (SEQ ID NO: 230). Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers.

“Fragment-based” bispecific antibodies or bispecific antibodies comprising a “single chain variable fragment” or “scFv” of this disclosure can refer to a single chain antibody, or fragment thereof, that comprises two binding moieties and a linker connecting the two binding moieties. The linker may be a polypeptide linker or other linker of suitable flexibility so as not to inhibit binding of either targeting moiety. Fragment based bispecific antibody formats include tandem VHH antibodies, tandem scFvs, scFv-Fabs, F(ab)₂, dual-affinity retargeting antibodies (DARTs). Such fragment-based antibodies can be further manipulated to comprise additional binding moieties with specificity for a given target e.g., A₂:B₁, A₁:B₂ or A₂:B₂, or with fragments of an Fc region to improve pharmacokinetics or promote ADCC, ADCP, or CDC.

A “binding moiety” refers to a portion of a molecule, peptide, polypeptide, antibody, or antibody fragment that mediates specific binding to a recited target or antigen or epitope. By way of example, the binding moiety of an antibody may comprise a heavy-chain/light-chain variable region pair or one or more complementarity determining regions (CDRs).

A “target” as referred to herein refers to the portion of a molecule that participates with a binding moiety of a molecule, peptide, polypeptide, antibody, or antibody fragment. A target can comprise an amino acid sequence and/or a carbohydrate, lipid or other chemical entity. An “antigen” is a target comprising a portion that is able to be bound by an adaptive immune molecule such as an antibody or antibody fragment, B-cell receptor, or T-cell receptor.

The “valency” of a bispecific or multi-specific molecule refers to the number of targets a recited molecule, peptide, polypeptide, antibody, or antibody fragment is able to bind. For instance, a molecule that is monovalent is able to bind to one molecule of a specific target, a bivalent molecule is able to bind to two molecules, and a tetravalent molecule is able to bind four targets. A bispecific, bivalent molecule, for example, is one that can bind to two targets and to two structurally different targets. For example, a bispecific, bivalent molecule when placed into contact with a solution comprising target A and target B may bind A₂, B₂ or A:B.

A “humanized” antibody is an antibody in which all or substantially all CDR amino acid residues are derived from non-human CDRs and all or substantially all FR amino acid residues are derived from human FRs. A humanized antibody optionally can include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

“ADCC” or “antibody dependent cell-mediated cytotoxicity” as used herein, refers to the cell-mediated reaction wherein nonspecific cytotoxic cells that express FcγRs recognize bound antibody on a target cell and subsequently cause lysis of the target cell. ADCC can be correlated with binding to FcγRIIIa wherein increased binding to FcγRIIIa leads to an increase in ADCC activity. “ADCP” or antibody dependent cell-mediated phagocytosis, as used herein, can refer to the cell-mediated reaction wherein nonspecific cytotoxic cells that express FcγRs recognize bound antibody on a target cell and subsequently cause phagocytosis of the target cell.

The terms “polypeptide” and “protein” are used interchangeably and refers to a polymer of amino acid residues, and are not limited to a minimum length. Polypeptides, including the provided antibodies and antibody chains and other peptides, e.g., linkers and binding peptides, can include amino acid residues including natural and/or non-natural amino acid residues. The terms also include post-expression modifications of the polypeptide, for example, glycosylation, sialylation, acetylation, phosphorylation, and the like. In some aspects, the polypeptides can contain modifications with respect to a native or natural sequence, as long as the protein maintains the desired activity. These modifications can be deliberate, as through site-directed mutagenesis, or can be accidental, such as through mutations of hosts which produce the proteins or errors due to PCR amplification.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence variants of the antibodies provided herein can be contemplated and conceived. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Antibody variants having one or more amino acid substitutions can be provided. Sites of interest for mutagenesis by substitution include the CDRs and FRs. Amino acid substitutions can be introduced into an antibody of interest and the products screened for a desired activity, e.g., retained/improved antigen binding, decreased immunogenicity, or improved ADCC or CDC.

This disclosure also provides for “immunoconjugates” or “antibody conjugates” or “antibody-drug conjugates” that refer to an antibody conjugated to one or more heterologous molecule(s). For example, an immunoconjugate can comprise an antibody conjugated to one or more cytotoxic agents, such as chemotherapeutic agents or drugs, growth inhibitory agents, protein domains, toxins (e.g., protein toxins, enzymatically active toxins of bacterial, fungal, plant, or animal origin, or fragments thereof), or radioactive isotopes. In some embodiments, an immunoconjugate can comprise the composite binding molecule disclosed herein, or fragment thereof (e.g., an scFv).

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215:403-410, 1990). Percent homology of sequences can be determined using the most recent version of BLAST, as of the filing date of this application.

The nucleic acids encoding the antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2 (5): 466-477. In certain embodiments, the cell is a Eukaryotic cell. In certain embodiments, the Eukaryotic cell is a mammalian cell. In certain embodiments, the mammalian cell is a cell line useful for producing antibodies is a Chines Hamster Ovary cell (CHO) cell, an NS0 murine myeloma cell, or a PER. C6® cell. In certain embodiments, the nucleic acid encoding the antibody is integrated into a genomic locus of a cell useful for producing antibodies. In certain embodiments, described herein is a method of making an antibody comprising culturing a cell comprising a nucleic acid encoding an antibody under conditions in vitro sufficient to allow production and secretion of said antibody.

As used herein the term “individual,” “patient,” or “subject” refers to individuals diagnosed with, suspected of being afflicted with, or at-risk of developing at least one disease for which the described compositions and method are useful for treating. In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

As used herein, the term “about” used to modify a specific number refers to that number plus or minus 10% of that number. The term “about” modifying a range refers to that range minus 10% of its lowest value and plus 10% of its greatest value.

As used herein, the terms “treatment” or “treating” are used in reference to a pharmaceutical or other intervention regimen used for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made. Skilled artisans will recognize that given a population of potential individuals for treatment not all will respond or respond equally to the treatment. Such individuals are considered treated.

The section headings used herein are for organizational purposes only and are not to be construed as limiting the subject matter described.

### Bispecific Molecules

Provided herein are bispecific or multivalent or composite binding molecules useful for treating cancers associated with CD19 positive, CD38 positive, CD20 negative B cells. Provided herein are bispecific or multivalent or composite binding molecules comprising a first binding component configured to bind a first target and a second binding component configured to bind a second target, wherein the first target comprises a B-cell lineage surface marker, and wherein the second target comprises a suppressive B-cell surface marker. Immunosuppressive B cells or B-cell populations can comprise a B-cell linage surface biomarker and a suppressive B-cell surface biomarker. The B-cell lineage surface markers can comprise CD19, CD20, CD138, IgA, or CD45. Immunosuppressive B-cell surface markers can comprise IgD, CD1, CD5, CD21, CD24, CD38, HM13, SLAMF7, AQP3, or latent TGF-beta (e.g., TGF-beta LAP). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38. In certain embodiments, the composite binding molecule binds to CD38 and CD19. In some embodiments, the suppressive B-cell surface marker comprises CD20. In certain embodiments, the suppressive B-cell surface marker consists of CD20.

A multivalent or bispecific or composite binding molecule possesses the ability to specifically bind to at least two structurally distinct targets. The specific binding may be the result of two distinct binding moieties that are structurally distinct at the molecular level, including but not limited to distinct non-identical amino acid sequences; or a single binding moiety that is able to specifically bind to two structurally distinct targets. A molecule, peptide, polypeptide, antibody, or antibody fragment referred to as “multi-specific” or “multivalent” or “bispecific” can refer to a molecule that possesses the ability to specifically bind to at least two structurally distinct targets. In some embodiments, the first or the second binding component of the composite binding molecule comprises a polypeptide. In certain embodiments, the first or the second binding component consists of a polypeptide. In some embodiments, the first and the second binding component of the composite binding molecule comprises a polypeptide. In certain embodiments, the first and the second binding component consist of a polypeptide. In certain embodiments, the polypeptide of the first or second binding component comprises an amino acid sequence at least 100 amino acid residues in length. In certain embodiments, the polypeptide of the first and second binding component comprise an amino acid sequence at least 100 amino acid residues in length.

A bispecific molecule can be a bispecific antibody that preserves at least one fragment of an antibody able to specifically bind with a target, for example, a variable region, heavy or light chain, or one or more complementarity determining regions from an antibody molecule. In some embodiments, the composite binding molecule described herein is a bispecific antibody and/or dual antigen-binding fragment thereof. Bispecific antibodies possess the ability to bind to two structurally distinct targets or antigens. In some embodiments, the bispecific antibody comprises a first binding component configured to bind a first target and a second binding component configured to bind a second target, wherein the first target comprises a B-cell lineage surface marker (e.g. CD19, CD138, IgA, or CD45), and wherein the second target comprises a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

Immunosuppressive B cells or immunosuppressive B-cell lineage cells can comprise cell surface biomarkers CD19 and CD38. Further disclosed herein are bispecific antibodies that target CD19 and CD38. In some embodiments, the CD19 binding component comprises a variable heavy chain (VH) comprising SEQ ID NO: 1. In certain embodiments, the CD19 binding component comprises a VH CDR1 region comprising any one of SEQ ID NO: 11, SEQ ID NO: 12, SEQ ID NO: 13, SEQ ID NO: 14, or SEQ ID NO: 15. In certain embodiments, the CD19 binding component comprises a VH CDR2 region comprising any one of SEQ ID NO: 21, SEQ ID NO: 22, SEQ ID NO: 23, SEQ ID NO: 24, or SEQ ID NO: 25. In certain embodiments, the CD19 binding component comprises a VH CDR3 region comprising any one of SEQ ID NO: 31, SEQ ID NO: 32, SEQ ID NO: 33, SEQ ID NO: 34, or SEQ ID NO: 35.

In some embodiments, the CD19 binding component comprises a variable light chain (VL) comprising SEQ ID NO: 2. In certain embodiments, the CD19 binding component comprises a VL CDR1 region comprising any one of SEQ ID NO: 41, SEQ ID NO: 42, SEQ ID NO: 43, SEQ ID NO: 45, or SEQ ID NO: 45. In certain embodiments, the CD19 binding component comprises a VL CDR2 region comprising any one of SEQ ID NO: 51, SEQ ID NO: 52, SEQ ID NO: 53, SEQ ID NO: 54, or SEQ ID NO: 55. In certain embodiments, the CD19 binding component comprises a VL CDR3 region comprising any one of SEQ ID NO: 61, SEQ ID NO: 62, SEQ ID NO: 63, SEQ ID NO: 64, or SEQ ID NO: 65.

In some embodiments, the bispecific antibody comprises a first binding component, wherein the first binding component comprises an HCDR1 amino acid sequence set forth in any one of SEQ ID NOs: 11-15, an HCDR2 amino acid sequence set forth in any one of SEQ ID NOs: 21-25, an HCDR3 amino acid sequence set forth in any one of SEQ ID NOs: 31-35, an LCDR1 amino acid sequence set forth in any one of SEQ ID NOs: 41-45, an LCDR2 amino acid sequence set forth in any one of SEQ ID NOs: 51-55, and/or an LCDR3 amino acid sequence set forth in any one of SEQ ID NOs: 61-65.

In some embodiments, the bispecific antibody comprises a CD19 binding component, wherein the CD19 binding component comprises an HCDR1 amino acid sequence set forth in SEQ ID NO: 11, an HCDR2 amino acid sequence set forth in SEQ ID NO: 21, an HCDR3 amino acid sequence set forth in SEQ ID NO: 31, an LCDR1 amino acid sequence set forth in SEQ ID NO: 41, an LCDR2 amino acid sequence set forth in SEQ ID NO: 51, and/or an LCDR3 amino acid sequence set forth in SEQ ID NO: 61.

In some embodiments, the bispecific antibody comprises a CD19 binding component, wherein CD19 first binding component comprises an HCDR1 amino acid sequence set forth in SEQ ID NO: 12, an HCDR2 amino acid sequence set forth in SEQ ID NO: 22, an HCDR3 amino acid sequence set forth in SEQ ID NO: 32, an LCDR1 amino acid sequence set forth in SEQ ID NO: 42, an LCDR2 amino acid sequence set forth in SEQ ID NO: 52, and/or an LCDR3 amino acid sequence set forth in SEQ ID NO: 62.

In some embodiments, the bispecific antibody comprises a CD19 binding component, wherein the CD19 binding component comprises an HCDR1 amino acid sequence set forth in SEQ ID NO: 15, an HCDR2 amino acid sequence set forth in SEQ ID NO: 25, an HCDR3 amino acid sequence set forth in SEQ ID NO: 35, an LCDR1 amino acid sequence set forth in SEQ ID NO: 45, an LCDR2 amino acid sequence set forth in SEQ ID NO: 55, and/or an LCDR3 amino acid sequence set forth in SEQ ID NO: 65.

In some embodiments, the CD19 binding comprises a variable heavy chain and light chain or CDRs corresponding to or derived from Inebilizumab, Tafasitamab, Taplitumomab, Obexelimab, Blinatumomab, Coltuximab, Denintuzumab, or Loncastuximab, MOR208, MEDI-551, XmAb 5871, MDX-1342, or AFM11.

In some embodiments, the CD38 binding component comprises a variable heavy chain (VH) comprising SEQ ID NO: 3. In certain embodiments, the CD19 binding component comprises a VH CDR1 region comprising any one of SEQ ID NO: 71, SEQ ID NO: 72, SEQ ID NO: 73, SEQ ID NO: 75, or SEQ ID NO: 75. In certain embodiments, the CD19 binding component comprises a VH CDR2 region comprising any one of SEQ ID NO: 81, SEQ ID NO: 82, SEQ ID NO: 83, SEQ ID NO: 84, or SEQ ID NO: 85. In certain embodiments, the CD19 binding component comprises a VH CDR3 region comprising any one of SEQ ID NO: 91, SEQ ID NO: 92, SEQ ID NO: 93, SEQ ID NO: 94, or SEQ ID NO: 95.

In some embodiments, the CD38 binding component comprises a variable light chain (VL) comprising SEQ ID NO: 4. In certain embodiments, the CD19 binding component comprises a VL CDR1 region comprising any one of SEQ ID NO: 101, SEQ ID NO: 102, SEQ ID NO: 103, SEQ ID NO: 105, or SEQ ID NO: 105. In certain embodiments, the CD19 binding component comprises a VL CDR2 region comprising any one of SEQ ID NO: 111, SEQ ID NO: 112, SEQ ID NO: 113, SEQ ID NO: 114, or SEQ ID NO: 115. In certain embodiments, the CD19 binding component comprises a VL CDR3 region comprising any one of SEQ ID NO: 121, SEQ ID NO: 122, SEQ ID NO: 123, SEQ ID NO: 124, or SEQ ID NO: 125.

In some embodiments, the bispecific antibody comprises a CD38 binding component, wherein the CD38 binding component comprises an HCDR1 amino acid sequence set forth in SEQ ID NO: 71, an HCDR2 amino acid sequence set forth in SEQ ID NO: 81, an HCDR3 amino acid sequence set forth in SEQ ID NO: 91, an LCDR1 amino acid sequence set forth in SEQ ID NO: 101, an LCDR2 amino acid sequence set forth in SEQ ID NO: 111, and/or an LCDR3 amino acid sequence set forth in SEQ ID NO: 121.

In some embodiments, the bispecific antibody comprises a CD38 binding component, wherein the CD38 binding component comprises an HCDR1 amino acid sequence set forth in SEQ ID NO: 72, an HCDR2 amino acid sequence set forth in SEQ ID NO: 82, an HCDR3 amino acid sequence set forth in SEQ ID NO: 92, an LCDR1 amino acid sequence set forth in SEQ ID NO: 102, an LCDR2 amino acid sequence set forth in SEQ ID NO: 112, and/or an LCDR3 amino acid sequence set forth in SEQ ID NO: 122.

In some embodiments, the bispecific antibody comprises a CD38 binding component, wherein the CD38 binding component comprises an HCDR1 amino acid sequence set forth in SEQ ID NO: 75, an HCDR2 amino acid sequence set forth in SEQ ID NO: 85, an HCDR3 amino acid sequence set forth in SEQ ID NO: 95, an LCDR1 amino acid sequence set forth in SEQ ID NO: 105, an LCDR2 amino acid sequence set forth in SEQ ID NO: 115, and/or an LCDR3 amino acid sequence set forth in SEQ ID NO: 125.

In some embodiments (e.g., any of the preceding embodiments), the CDR-H2 of the CD38 binding component comprises the amino acid residues P(X1)LG(X2)A (SEQ ID NO: 150), wherein X1 and X2 tolerate amino acid substitutions while maintaining binding to CD38. In certain embodiments, X1 and X2 are selected from amino acids that reduce the hydrophobicity of the CDRH2 amino acid sequence. In certain embodiments, the amino acids that reduce the hydrophobicity include H, Q, T, N, S, G, A, R, K, D, or E. In certain embodiments, the X1 is H and X2 is T.

In some embodiments, the bispecific antibody comprises a CD38 binding component and a CD19 binding component, wherein the CD38 binding component comprises a VH amino acid sequence and a VL amino acid sequence and, wherein the VH amino acid sequence comprises an amino acid sequence at least about 90%, 95%, 97%, 98%, or 99% identical to SEQ ID NO: 3, and the VL comprises an amino acid sequence at least about 90%, 95%, 97%, 98%, or 99% identical to SEQ ID NO: 4; and the CD19 binding component comprises a VH amino acid sequence and a VL amino acid sequence, wherein the VH amino acid sequence comprises an amino acid sequence at least about 90%, 95%, 97%, 98%, or 99% identical to SEQ ID NO: 1, and the VL comprises an amino acid sequence at least about 90%, 95%, 97%, 98%, or 99% identical to SEQ ID NO: 2.

In some embodiments, the bispecific antibody comprises a CD38 binding component and a CD19 binding component, wherein the CD38 binding component comprises a VH amino acid sequence and a VL amino acid sequence and, wherein the VH amino acid sequence comprises an amino acid sequence identical to SEQ ID NO: 3, and the VL comprises an amino acid sequence identical to SEQ ID NO: 4; and the CD19 binding component comprises a VH amino acid sequence and a VL amino acid sequence, wherein the VH amino acid sequence comprises an amino acid sequence identical to SEQ ID NO: 1, and the VL comprises an amino acid sequence identical to SEQ ID NO: 2.

In some embodiments, the bispecific antibody comprises a CD38 binding component and a CD19 binding component, wherein the CD38 binding component comprises a VH amino acid sequence and a VL amino acid sequence and, wherein the VH amino acid sequence comprises an amino acid sequence at least about 90%, 95%, 97%, 98%, or 99% identical to SEQ ID NOs: 3, 215, or 218-223, and the VL comprises an amino acid sequence at least about 90%, 95%, 97%, 98%, or 99% identical to SEQ ID NO:s 4 or 223; and the CD19 binding component comprises a VH amino acid sequence and a VL amino acid sequence, wherein the VH amino acid sequence comprises an amino acid sequence at least about 90%, 95%, 97%, 98%, or 99% identical to SEQ ID NOs: 1, 201, or 216-217, and the VL comprises an amino acid sequence at least about 90%, 95%, 97%, 98%, or 99% identical to SEQ ID NO: 2. In some embodiments, the CD19 binding component comprises a VH amino acid sequence comprising a substitution at A84 and A108. In some embodiments, the substitution comprises A84S and A108L.

In some embodiments, the bispecific antibody comprises a CD38 binding component and a CD19 binding component, wherein the CD38 binding component comprises a VH amino acid sequence and a VL amino acid sequence and, wherein the VH amino acid sequence comprises an amino acid sequence identical to SEQ ID NO: 3, 215, or 218-223, and the VL comprises an amino acid sequence identical to SEQ ID NO: 4 or 223; and the CD19 binding component comprises a VH amino acid sequence and a VL amino acid sequence, wherein the VH amino acid sequence comprises an amino acid sequence identical to SEQ ID NO: 1, 201, 216-217 and the VL comprises an amino acid sequence identical to SEQ ID NO: 2. In some embodiments, the CD19 binding component comprises a VH amino acid sequence comprising a substitution at A84 and A108. In some embodiments, the substitution comprises A84S and A108L.

In some embodiments, the bispecific antibody comprises a CD38 binding component and a CD19 binding component, wherein the CD38 binding component comprises an HCDR1 amino acid sequence set forth in SEQ ID NO: 71, an HCDR2 amino acid sequence set forth in SEQ ID NO: 81, an HCDR3 amino acid sequence set forth in SEQ ID NO: 91, an LCDR1 amino acid sequence set forth in SEQ ID NO: 101, an LCDR2 amino acid sequence set forth in SEQ ID NO: 111, and/or an LCDR3 amino acid sequence set forth in SEQ ID NO: 121; and the CD19 binding component comprises an HCDR1 amino acid sequence set forth in SEQ ID NO: 11, an HCDR2 amino acid sequence set forth in SEQ ID NO: 21, an HCDR3 amino acid sequence set forth in SEQ ID NO: 31, an LCDR1 amino acid sequence set forth in SEQ ID NO: 41, an LCDR2 amino acid sequence set forth in SEQ ID NO: 51, and/or an LCDR3 amino acid sequence set forth in SEQ ID NO: 61.

In some embodiments, the bispecific antibody comprises a CD38 binding component and a CD19 binding component, wherein the CD38 binding component comprises an HCDR1 amino acid sequence set forth in SEQ ID NO: 72, an HCDR2 amino acid sequence set forth in SEQ ID NO: 82, an HCDR3 amino acid sequence set forth in SEQ ID NO: 92, an LCDR1 amino acid sequence set forth in SEQ ID NO: 102, an LCDR2 amino acid sequence set forth in SEQ ID NO: 112, and/or an LCDR3 amino acid sequence set forth in SEQ ID NO: 122; and the CD19 binding component comprises an HCDR1 amino acid sequence set forth in SEQ ID NO: 12, an HCDR2 amino acid sequence set forth in SEQ ID NO: 22, an HCDR3 amino acid sequence set forth in SEQ ID NO: 32, an LCDR1 amino acid sequence set forth in SEQ ID NO: 42, an LCDR2 amino acid sequence set forth in SEQ ID NO: 52, and/or an LCDR3 amino acid sequence set forth in SEQ ID NO: 62.

In some embodiments, the bispecific antibody comprises a CD38 binding component and a CD19 binding component, wherein the CD38 binding component comprises an HCDR1 amino acid sequence set forth in SEQ ID NO: 75, an HCDR2 amino acid sequence set forth in SEQ ID NO: 85, an HCDR3 amino acid sequence set forth in SEQ ID NO: 95, an LCDR1 amino acid sequence set forth in SEQ ID NO: 105, an LCDR2 amino acid sequence set forth in SEQ ID NO: 115, and/or an LCDR3 amino acid sequence set forth in SEQ ID NO: 125; and the CD19 binding component comprises an HCDR1 amino acid sequence set forth in SEQ ID NO: 15, an HCDR2 amino acid sequence set forth in SEQ ID NO: 25, an HCDR3 amino acid sequence set forth in SEQ ID NO: 35, an LCDR1 amino acid sequence set forth in SEQ ID NO: 45, an LCDR2 amino acid sequence set forth in SEQ ID NO: 55, and/or an LCDR3 amino acid sequence set forth in SEQ ID NO: 65.

In some embodiments, the CD38 binding comprises a variable heavy chain and light chain or CDRs corresponding to or derived from Daratumumab or Isatuximab.

Substitutions, insertions, or deletions may occur within one or more CDRs, wherein the substitutions, insertions, or deletions do not substantially reduce antibody binding to antigen. For example, conservative substitutions that do not substantially reduce binding affinity may be made in CDRs. Such alterations may be outside of CDR “hotspots”. In some embodiments, of the variant VH and VL sequences, each CDR is unaltered. Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Antibodies can be altered to increase or decrease their glycosylation (e.g., by altering the amino acid sequence such that one or more glycosylation sites are created or removed). A carbohydrate attached to an Fc region of an antibody may be altered. Native antibodies from mammalian cells typically comprise a branched, biantennary oligosaccharide attached by an N-linkage to Asn₂₉₇ of the CH2 domain of the Fc region (See e.g., Wright et al. TIBTECH 15:26-32 (1997)). The oligosaccharide can be various carbohydrates, e.g., mannose, N-acetyl glucosamine (GlcNAc), galactose, sialic acid, fucose attached to a GlcNAc in the stem of the biantennar oligosaccharide structure. Modifications of the oligosaccharide in an antibody can be made, for example, to create antibody variants with certain improved properties. Antibody glycosylation variants can have improved ADCC and/or CDC function. In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn₂₉₇, relative to the sum of all glycostructures attached to Asn297 (See e.g., WO 08/077546). Asn₂₉₇ refers to the asparagine residue located at about position 297 in the Fc region (EU numbering of Fc region residues; See e.g., Edelman et al. Proc Natl Acad Sci USA. 1969 May; 63 (1): 78-85). However, Asn₂₉₇ may also be located about ±3 amino acids upstream or downstream of position 297, i.e., between positions 294 and 300, due to minor sequence variations in antibodies. Such fucosylation variants can have improved ADCC function (See e.g., Okazaki et al. J. Mol. Biol. 336:1239-1249 (2004); and Yamane-Ohnuki et al. Biotech. Bioeng. 87:614 (2004)). Cell lines, e.g., knockout cell lines and methods of their use can be used to produce defucosylated antibodies, e.g., Lec13 CHO cells deficient in protein fucosylation and alpha-1,6-fucosyltransferase gene (FUT8) knockout CHO cells (See e.g., Ripka et al. Arch. Biochem. Biophys. 249:533-545 (1986); Yamane-Ohnuki et al. Biotech. Bioeng. 87:614 (2004); Kanda, Y. et al., Biotechnol. Bioeng., 94 (4): 680-688 (2006)). Other antibody glycosylation variants are also included (See e.g., U.S. Pat. No. 6,602,684).

In some embodiments, the composite binding molecule provided herein has a dissociation constant (K_{D}) of about 10 μM, 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or less (e.g., 10⁻⁸ M or less, e.g., from 10⁻⁸ M to 10⁻¹³ M, e.g., from 10⁻⁹ M to 10⁻¹³ M) for the antibody target. The antibody target can be a CD19 target, a CD38 target, or a target comprising both CD19 and CD38. K_{D} can be measured by any suitable assay. In certain embodiments, KD can be measured using surface plasmon resonance assays (e.g., using a BIACORE®-2000 or a BIACORE®-3000 or Octet).

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody provided herein may be further modified to contain additional non-proteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

Composite binding molecules or bispecific antibodies can differ based on the binding moieties associated with these molecules, wherein there are also several different formats that can be deployed and are envisioned herein. Composite binding molecules or bispecific antibodies can comprise on antibody fragments, substantially intact antibodies, or a combination thereof. In some embodiments, the first or second binding component comprises an immunoglobulin heavy and light chain pair, an scFv, a F(ab), a F(ab′)₂, a single domain antibody, a variable region fragment from an immunoglobulin new antigen receptor (VNAR), or a variable region derived from a heavy chain antibody (VHH). In certain embodiments, the first and second binding component comprise an immunoglobulin heavy and light chain pair, an scFv, a F(ab), a F(ab′)₂, a single domain antibody, a variable region fragment from an immunoglobulin new antigen receptor (VNAR), or a variable region derived from a heavy chain antibody (VHH). In some embodiments, the first or second binding component comprises an immunoglobulin heavy and light chain pair. In certain embodiments, the first and second binding component comprise an immunoglobulin heavy and light chain pair. In some embodiments, the first or second binding component comprises an scFv. In certain embodiments, the first and second binding component comprise an scFv.

Bispecific antibodies according to this disclosure comprise intact antibody molecules or substantially fully intact antibody molecules, and may be asymmetric or symmetric.

Asymmetric bispecific antibodies generally comprise a heavy chain/light chain (HC/LC) pair from an antibody specific for target A and an HC/LC pair from an antibody specific for target B, creating a hetero-bifunctional antibody. Hetero-bifunctional antibodies such as these face the problem of unproductive formation of the molecule when it is being produced. HC/LC-A: HC/LC-B is desired, but is usually thermodynamically or statistically unfavorable from all the possible combinations possible. Multiple schemes have been introduced to circumvent this problem. In some instances, the HC/LC pair from an antibody with specificity for A and the HC/LC pair from an antibody with specificity for B further comprise mutations to the FC region to increase the probability of formation of an antibody with HC/LC-A: HC/LC-B. This can be achieved by engineering structural features such as “knobs” into the FC region for HC-A, and “holes” into HC-B, or vice versa, that promote formation of heterodimers between HC-A and HC-B. Another scheme to promote HC-A: HC-B heterodimers is to engineer amino acid residues in the FC portion of HC-A and HC-B to comprise charge pairs that favor electrostatic interactions between HC-B and HC-A. Another scheme to address the problem of chain association is to replace the variable regions of one of the HC/LC pairs with a single-chain binding molecules (e.g., VHH or an scFv). Such that one-half of the molecule comprises a classical HC/LC pair and the other comprises a HC constant region fused or otherwise connected to the single-chain binding molecule. Further modifications can be made to promote proper HC/LC paring and include engineering mutations to the HC and LC for either A or B to favor formation of the proper HC/LC pair; CrossMab technology, which entails swapping the corresponding constant regions of the HC/LC pair. Symmetric bispecific antibodies circumvent the chain association problem by not relying on formation of a hetero-bifunctional molecule. Such examples include: the dual-variable domain molecule, which comprises stacked variable regions of differing specificity; the IgG-scFv molecule, which comprises an scFv of a differing specificity fused to the c-terminus of heavy chain of a classical antibody molecule; the (scFV)₄-FC, which comprises two scFvs connected by an Fc region of an Ig (the Fcs dimerize creating a bispecific, tetravalent molecule); the DART-Fc and the two-in-one, amongst others.

The structure of composite binding molecules or bispecific antibodies can be conceived and designed to alter functionality or binding properties of the composite binding molecules or bispecific antibodies (see e.g., “Bispecific antibodies: a mechanistic review of the pipeline.” Nat Rev Drug Discovery. 2019 August; 18 (8): 585-608) (see e.g., “The making of bispecific antibodies” MAbs. 2017 February-March; 9 (2): 182-212). For example, the bispecific antibody can be selected from one of the following formats: a common light chain bispecific IgG, a Fab-Fc:scFv-Fc bispecific IgG, a Fab-Fc-Fab:Fc bispecific IgG, a Fab-Fc-scFv:Fab-Fc-scFv bispecific IgG, a Fab-Fc-scFv:Fc bispecific IgG, a Fab-Fc-Fab:Fab-Fc bispecific IgG, an scFv-Fab-Fc:scFv-Fab-Fc bispecific IgG, a Fab-Fab-Fc:Fab-Fab-Fc bispecific IgG, a Fab-Fc-Fab:Fab-Fc-Fab bispecific IgG, and a Fab-Fc-scFv:Fab-Fc bispecific IgG.

### Common Light Chain Bispecific IgG

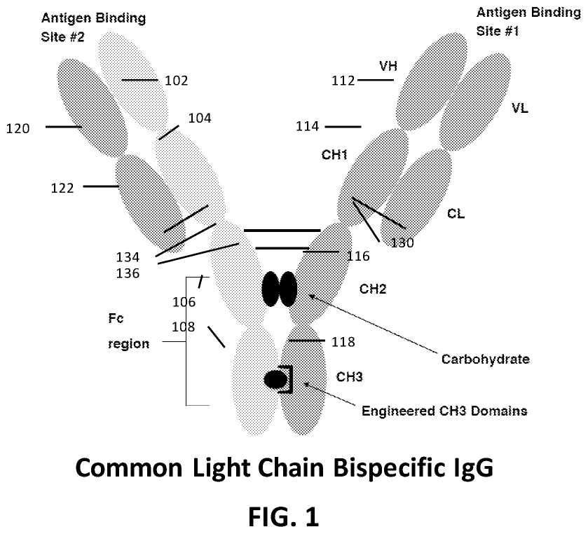

A bispecific antibody having a common light chain bispecific IgG structure can be used herein. FIG. 1 illustrates a bispecific antibody having a common light chain bispecific IgG structure. The structure comprises a first and a second IgG heavy chain. Each heavy chain comprises a VH, CH1, CH2, and CH3 domain. The first heavy chain comprises VH 102, CH1 104, CH2 106, and CH3 108. The second heavy chain comprises VH 112, CH1 114, CH2 116, and CH3 118. The common light chain bispecific IgG structure also comprises a light chain comprising a VL domain 120 and a CL domain 122. Generally, the first heavy chain will comprise a sequence derived from the heavy chain of an antibody with a first specificity; and the second heavy chain will comprise a heavy chain from an antibody with a second specificity. The light chain that pairs with the first and the second heavy chain will be identical, and can be derived from the light chain of an antibody with either specificity, or a separate specificity. A heavy chain can be covalently coupled to a light chain molecule via a covalent bond (e.g. disulfide bond 130). A heavy chain can be coupled to another heavy chain via one or more covalent bonds (e.g. disulfide bond 134 and/or 136). The common light chain bispecific IgG structure can comprise a first and a second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance, “knobs” into “holes”) or biochemically (e.g. electrostatic interactions) prevent coupling of the two first heavy chain molecules or two second heavy chain molecules. Exemplary knob into hole mutations can comprise T366W (EU numbering) in one heavy chain and T366S/L368A/Y407V (EU numbering) in a second heavy chain. Exemplary mutations that facilitate coupling of a first and a second heavy chain molecule are disclosed, for example in WO2009089004, U.S. Pat. No. 8,642,745, US PG-PUB: US20140322756 and “The making of bispecific antibodies” MAbs. 2017 February-March; 9 (2): 182-212. The common light chain bispecific IgG structure can also comprise carbohydrate molecules 140 coupled thereto or additional modifications thereof.

A bispecific antibody having a common light chain bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the first heavy chain is configured to bind B-cell lineage surface marker and the second heavy is configured to bind a suppressive B-cell surface marker. In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

In some embodiments, the first heavy chain comprises a VH sequence comprising a CD19 binding component and the second heavy chain comprises a VH sequence comprising CD38 binding component. In certain embodiments, the heavy chain CD19 binding component comprises SEQ ID NO: 201, 1, or a variant comprising a mutation at one or both of A84 and A108 of SEQ ID NO: 201 and the heavy chain CD38 binding component comprises SEQ ID NOs: 202, 215, 218-221. In certain embodiments, the variant comprises the mutation A84S and A108L. In some embodiments, the bispecific antibody comprises a common light chain. In certain embodiments, the common light chain sequence comprises a CD19 binding component (e.g. SEQ ID NO: 2). In certain embodiments, the common light chain sequence comprises CD38 binding component (e.g. SEQ ID NO: 4 or SEQ ID NO: 222).

Described herein BS1 comprises a common light chain format with a CD19 binding component configured to bind CD19 and a CD38 binding component configured to bind CD38, wherein the CD19 binding component comprises an antibody or antigen binding fragment thereof and the CD38 binding component comprises an antibody or antigen binding fragment thereof, wherein the CD38 antibody or antigen binding fragment comprises an anti-CD38 immunoglobulin heavy chain variable region paired with an anti-CD38 immunoglobulin light chain variable region and the CD19 antibody or antigen binding fragment comprises an anti-CD19 immunoglobulin heavy chain variable region paired with an anti-CD38 immunoglobulin light chain variable region, wherein the CD38 antibody or antigen binding component comprises: a) a heavy chain complementarity determining region 1 (HCDR1) comprising an amino acid sequence set forth in any one of SEQ ID NOs: 71-75, b) a heavy chain complementarity determining region 2 (HCDR2) comprising an amino acid sequence set forth in any one of SEQ ID NOs: 81-85, or 150-155; c) a heavy chain complementarity determining region 3 (HCDR3) comprising an amino acid sequence set forth in any one of SEQ ID NOs: 91-95; d) a light chain complementarity determining region 1 (LCDR1) comprising an amino acid sequence set forth in any one of SEQ ID NOs: 101-105; e) a light chain complementarity determining region 2 (LCDR2) comprising an amino acid sequence set forth in any one of SEQ ID NOs: 111-115; and/or) a light chain complementarity determining region 3 (LCDR3) comprising an amino acid sequence set forth in any one of SEQ ID NOs: 121-125; and wherein the CD19 antigen binding component comprises: g) a heavy chain complementarity determining region 1 (HCDR1) comprising an amino acid sequence set forth in any one of SEQ ID NOs: 11-15, h) a heavy chain complementarity determining region 2 (HCDR2) comprising an amino acid sequence set forth in any one of SEQ ID NOs: 21-25, i) a heavy chain complementarity determining region 3 (HCDR3) comprising an amino acid sequence set forth in any one of SEQ ID NOs: 31-35; j) a light chain complementarity determining region 1 (LCDR1) comprising an amino acid sequence set forth in any one of SEQ ID NOs: 101-105; k) a light chain complementarity determining region 2 (LCDR2) comprising an amino acid sequence set forth in any one of SEQ ID NOs: 111-115; and/or 1) a light chain complementarity determining region 3 (LCDR3) comprising an amino acid sequence set forth in any one of SEQ ID NOs: 121-125. In some embodiments, the CD38 antigen binding component comprises a HCDR2 amino acid sequence comprising the sequence P-X1-L-G-X2-A (SEQ ID NO: 156), wherein X1 and X2 are each selected from the group consisting of H, Q, T, N, S, G, A, R, K, D, or E. In certain embodiments, the X1 is H and X2 is T. In some embodiments, the CD19 heavy chain sequence comprises a A84S and/or A108L substitution. In some embodiments, the CD38 light chain comprises a W32H substitution.

Fab-Fc:scFv-Fc Bispecific IgG

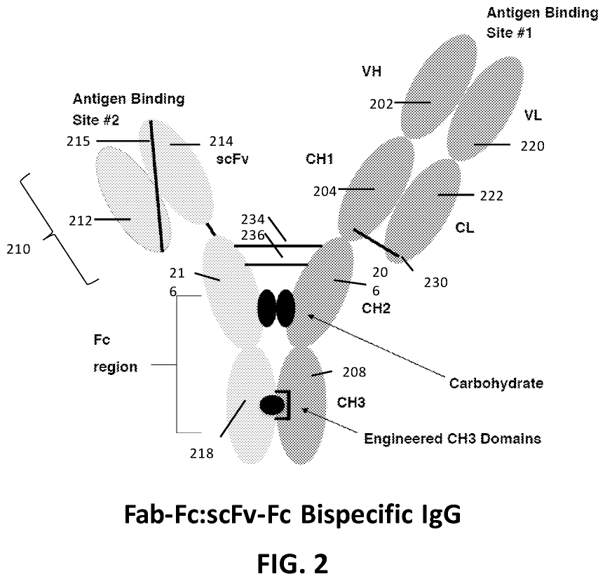

A bispecific antibody having a Fab-Fc:scFv-Fc Bispecific IgG structure can be used herein. FIG. 2 illustrates a bispecific antibody having a Fab-Fc:scFv-Fc Bispecific IgG structure. The structure comprises a first heavy chain molecule and a modified second IgG heavy chain molecule comprising a single chain variable fragment. The first heavy chain comprises VH 202, CH1 204, CH2 206, and CH3 208, N-terminus to C-terminus respectively. The modified second heavy chain comprises a single chain variable fragment (scFv) 210, CH2 216, and CH3 218, N-terminus to C-terminus respectively. The single chain variable fragment (scFv) can comprises a first domain 212 corresponding to a variable light chain domain, or fragment thereof, a second domain 214 corresponding to a variable heavy chain, or a fragment thereof, and a linker polypeptide 215. The Fab-Fc:scFv-Fc Bispecific IgG structure also comprises a light chain comprising a VL domain 220 and a CL domain 222. The first heavy chain can be covalently coupled to a light chain molecule via a covalent bond (e.g. disulfide bond 230). A first heavy chain can be coupled to the modified second heavy chain via one or more covalent bonds (e.g. disulfide bond 234 and/or 236). The Fab-Fc:scFv-Fc Bispecific IgG structure can comprise a first and a modified second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance) or biochemically (e.g. electrostatic interactions) prevent coupling of the two first heavy chain molecules or two second heavy chain molecules. Exemplary mutations that facilitate coupling of a first and a second heavy chain molecule are disclosed, for example in US PG-PUB: US20140322756 and “The making of bispecific antibodies” MAbs. 2017 February-March; 9 (2): 182-212. The Fab-Fc:scFv-Fc Bispecific IgG structure can also comprise carbohydrate molecules 240 coupled thereto or additional modifications thereof.

A bispecific antibody having a Fab-Fc:scFv-Fc Bispecific IgG structure can target a B-cell lineage surface marker (e.g. CD19, CD138, IgA, or CD45e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc:scFv-Fc Bispecific IgG structure can be engineered so that a first antigen binding site targets CD19 and a second antigen binding site targets CD38. In some embodiments, the first heavy chain comprises a VH sequence comprising CD19 binding component and the second heavy chain comprises a single chain variable fragment (scFv) sequence comprising a CD38 binding component. In certain embodiments, the heavy chain comprising the CD38 single chain variable fragment comprises SEQ ID NO: 205 or SEQ ID NO: 206. In certain embodiments, the VL sequence comprises a CD19 binding component. In certain embodiments, the single chain variable fragment (scFv) sequence comprising a CD38 binding component comprises a CD38 binding component corresponding to an antibody heavy chain and light variable sequence, or CD38 binding fragment thereof. In some embodiments, the first heavy chain comprises a VH sequence comprising CD38 binding component and the second heavy chain comprises a single chain variable fragment (scFv) sequence comprising a CD19 binding component. In certain embodiments, the heavy chain comprising the CD19 single chain variable fragment comprises SEQ ID NO: 203 or SEQ ID NO: 204 or SEQ ID NO: 217. In certain embodiments, the single chain variable fragment (scFv) sequence comprising a CD19 binding component comprises a CD19 binding component corresponding to an antibody heavy chain and light variable sequence, or CD19 binding fragment thereof.

The Fab-Fc:scFv-Fc Bispecific IgG structure can be engineered so that a first antigen binding site targets CD38 and a second antigen binding site targets CD19. In some embodiments, the first heavy chain comprises a VH sequence comprising CD38 binding component and the second heavy chain comprises a single chain variable fragment (scFv) sequence comprising a CD19 binding component. In certain embodiments, the VL sequence comprises a CD38 binding component. In certain embodiments, the single chain variable fragment (scFv) sequence comprising a CD19 binding component comprises a CD19 binding component corresponding to an antibody heavy chain and light variable sequence, or CD19 binding fragment thereof.

Described herein BS2 comprises a CD19 binding component configured to bind CD19 and a CD38 binding component configured to bind CD38, wherein the CD19 binding component comprises an antibody or antigen binding fragment thereof and the CD38 binding component comprises an antibody or antigen binding fragment thereof, wherein the CD38 antigen binding component comprises a Fab that binds CD38 comprising an anti-CD38 immunoglobulin heavy chain variable region paired with an anti-CD38 immunoglobulin light chain variable region and the CD19 antigen binding component comprises an scFv that binds CD19 comprising an anti-CD19 immunoglobulin heavy chain variable region paired with an anti-CD38 immunoglobulin light chain variable region, wherein the CD38 binding component comprises an immunoglobulin heavy chain comprising an HCDR1 amino acid sequence set forth in any one of SEQ ID NOs: 71-75, an HCDR2 amino acid sequence set forth in any one of SEQ ID NOs: 81-85, or 150-155, an HCDR3 amino acid sequence set forth in any one of SEQ ID NOs: 91-95; and the immunoglobulin light chain comprises an LCDR1 amino acid sequence set forth in any one of SEQ ID NOs: 101-105, an LCDR2 amino acid sequence set forth in any one of SEQ ID NOs: 111-115, and/or an LCDR3 amino acid sequence set forth in any one of SEQ ID NOs: 121-125; and wherein the CD19 binding component comprises an immunoglobulin heavy chain comprising an HCDR1 amino acid sequence set forth in any one of SEQ ID NOs: 11-15, an HCDR2 amino acid sequence set forth in any one of SEQ ID NOs: 21-25, an HCDR3 amino acid sequence set forth in any one of SEQ ID NOs: 31-35; and the immunoglobulin light chain comprises an LCDR1 amino acid sequence set forth in any one of SEQ ID NOs: 41-45, an LCDR2 amino acid sequence set forth in any one of SEQ ID NOs: 51-55, and/or an LCDR3 amino acid sequence set forth in any one of SEQ ID NOs: 61-65. In some embodiments, the CD 38 antigen binding component comprises a HCDR2 amino acid sequence comprising the sequence P-X1-L-G-X2-A (SEQ ID NO: 156), wherein X1 and X2 are selected from the group consisting of H, Q, T, N, S, G, A, R, K, D, or E. In certain embodiments, the X1 is Hand X2 is T. In some embodiments, the CD19 heavy chain sequence comprises a A84S and/or A108L substitution. In some embodiments, the CD38 light chain comprises a W32H substitution.

### Fab-Fc-Fab:Fc Bispecific IgG

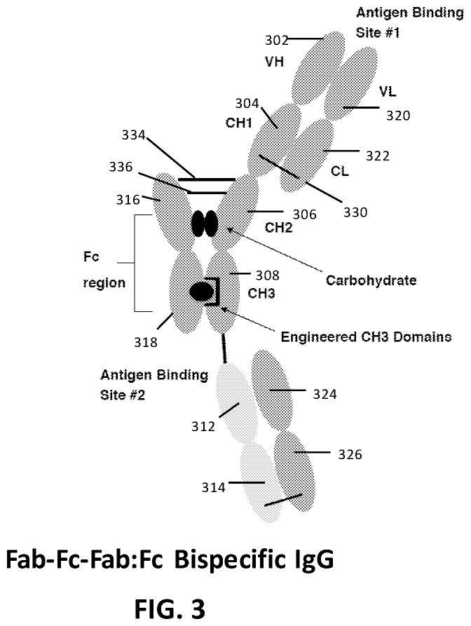

An engineered bispecific antibody having a Fab-Fc-Fab:Fc Bispecific IgG structure can be used herein. FIG. 3 illustrates a bispecific antibody having a Fab-Fc-Fab:Fc Bispecific IgG structure. The structure comprises a first heavy chain molecule and a modified IgG heavy chain molecule. The first heavy chain comprises VH domain 302, CH1 domain 304, CH2 domain 306, CH3 domain 308, a linker 310, a second VH domain 312, and a second CH1 domain 314, N-terminus to C-terminus respectively. The modified heavy chain comprises a CH2 domain 316, and CH3 domain 318, N-terminus to C-terminus respectively. The Fab-Fc-Fab:Fc Bispecific IgG structure also comprises a first light chain comprising a VL domain 320 and a CL domain 322. The Fab-Fc-Fab:Fc Bispecific IgG structure also comprises a second light chain comprising a VL domain 324 and a CL domain 326. A heavy chain can be covalently coupled to a light chain molecule via a covalent bond (e.g. disulfide bond 330). The first heavy chain can also be covalently coupled to the first second chain molecule via a covalent bond (e.g. disulfide bond 332). A heavy chain and a light chain can be coupled in a manner that the VH domain and CH1 domain of the first heavy chain pair with the VL domain and CL domain of the first light chain. The first heavy chain and second light chain can be coupled in a manner that the second VH domain and second CH1 domain of the first heavy chain pair with the VL domain and CL domain of the second light chain. The first heavy chain can be coupled to the modified second heavy chain via one or more covalent bonds (e.g. disulfide bond 334 and/or 336). The Fab-Fc-Fab:Fc Bispecific IgG structure can comprise a first and a modified second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance) or biochemically (e.g. electrostatic interactions) prevent coupling of the two first heavy chain molecules or two second heavy chain molecules. Exemplary mutations that facilitate coupling of a first and a second heavy chain molecule are disclosed, for example in US PG-PUB: US20140322756 and “The making of bispecific antibodies” MAbs. 2017 February-March; 9 (2): 182-212. The Fab-Fc-Fab:Fc Bispecific IgG structure can also comprise carbohydrate molecules 340 coupled thereto or additional modifications thereof.

A bispecific antibody having a Fab-Fc-Fab:Fc Bispecific IgG structure can target a B-cell lineage surface marker (e.g. CD19, CD138, IgA, or CD45e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc-Fab:Fc Bispecific IgG structure can be engineered so that a first antigen binding site targets CD19 and a second antigen binding site targets CD38. In some embodiments, the first heavy chain VH domain (e.g. 302) and VL domain (e.g. 320) comprises a CD19 binding component, wherein the second VH domain (e.g. 312) and VL domain (e.g. 324) comprises a CD38 binding component. In some embodiments, the Fab-Fc-Fab heavy chain comprises SEQ ID NO: 207 and the Fc heavy chain comprises SEQ ID NO: 208.

The Fab-Fc-Fab:Fc Bispecific IgG structure can also be engineered so that a first antigen binding site targets CD38 and a second antigen binding site targets CD19. In some embodiments, the first heavy chain V_{H} domain (e.g. 302) and V_{L} domain (e.g. 320) comprises a CD38 binding component, wherein the second V_{H} domain (e.g. 312) and V_{L} domain (e.g. 324) comprises a CD19 binding component.

### Fab-Fc-scFv:Fab-Fc-scFv Bispecific IgG

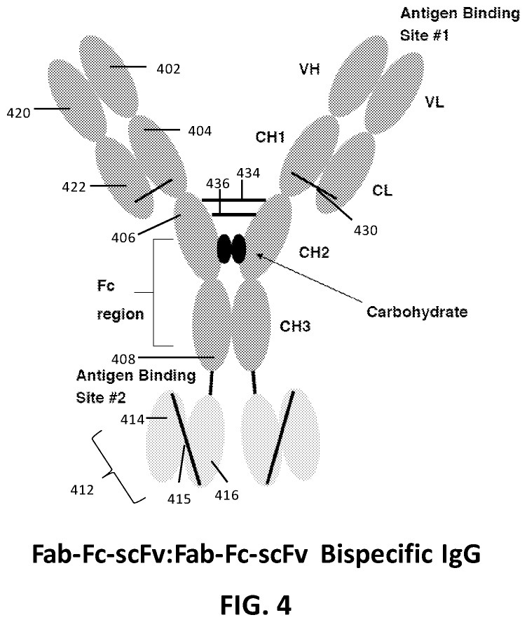

An engineered bispecific antibody having a Fab-Fc-scFv:Fab-Fc-scFv Bispecific IgG structure can be used herein. FIG. 4 illustrates a bispecific antibody having a Fab-Fc-scFv:Fab-Fc-scFv Bispecific IgG structure. The structure comprises a two first heavy chain molecules. The first heavy chain comprises VH domain 402, CH1 domain 404, CH2 domain 406, CH3 domain 408, a linker 410, and a single chain variable fragment (scFv) 412, N-terminus to C-terminus respectively. The single chain variable fragment (scFv) can comprises a first domain 414 corresponding to a variable light chain domain, or fragment thereof, a second domain 416 corresponding to a variable heavy chain, or a fragment thereof, and a second linker polypeptide 415. The Fab-Fc-scFv:Fab-Fc-scFv Bispecific IgG structure also comprises a first light chain comprising a VL domain 420 and a CL domain 422. A heavy chain can be covalently coupled to a light chain molecule via a covalent bond (e.g. disulfide bond 430). A heavy chain can be coupled to another heavy chain via one or more covalent bonds (e.g. disulfide bond 434 and/or 436). The Fab-Fc-scFv:Fab-Fc-scFv Bispecific IgG structure can also comprise carbohydrate molecules 440 coupled thereto or additional modifications thereof.

A bispecific antibody having a Fab-Fc-scFv:Fab-Fc-scFv Bispecific IgG structure can target a B-cell lineage surface marker (e.g. CD19, CD138, IgA, or CD45e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc-scFv:Fab-Fc-scFv Bispecific IgG structure can be engineered so that a first antigen binding site targets CD19 and a second antigen binding site targets CD38. In some embodiments, the first heavy chain VH domain (e.g. 402) and VL domain (e.g. 420) comprises a CD19 binding component, wherein the single chain variable fragment (scFv) (e.g. 412) sequence comprises a CD38 binding component. In certain embodiments, the single chain variable fragment (scFv) sequence comprising a CD38 binding component comprises a CD38 binding component corresponding to an antibody heavy chain and light variable sequence, or CD38 binding fragments thereof

The Fab-Fc-scFv:Fab-Fc-scFv Bispecific IgG structure can also be engineered so that a first antigen binding site targets CD38 and a second antigen binding site targets CD19. In some embodiments, the first heavy chain VH domain (e.g. 402) and VL domain (e.g. 420) comprises a CD38 binding component, wherein the single chain variable fragment (scFv) (e.g. 412) sequence comprises a CD19 binding component. In certain embodiments, the single chain variable fragment (scFv) sequence comprising a CD19 binding component comprises a CD19 binding component corresponding to an antibody heavy chain and light variable sequence, or CD19 binding fragments thereof. In some embodiments, the Fab-Fc-scFv heavy chain comprises SEQ ID NO: 209.

### Fab-Fc-scFv:Fc Bispecific IgG

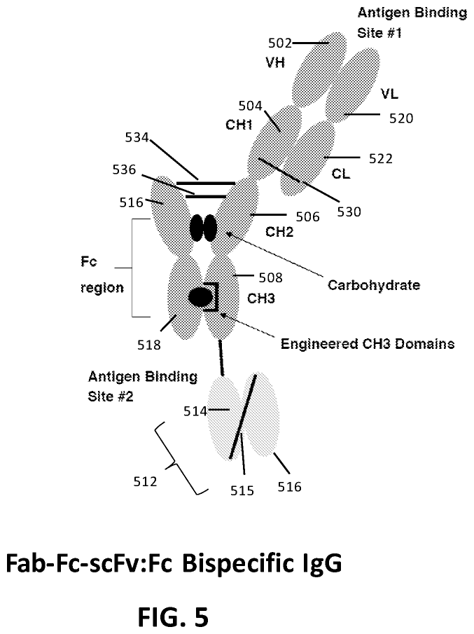

An engineered bispecific antibody having a Fab-Fc-scFv:Fc Bispecific IgG structure can be used herein. FIG. 5 illustrates a bispecific antibody having a Fab-Fc-scFv:Fc Bispecific IgG structure. The structure comprises a first heavy chain molecule and a second IgG heavy chain molecule. The first heavy chain comprises VH domain 502, CH1 domain 504, CH2 domain 506, CH3 domain 508, a linker 510, and a single chain variable fragment (scFv) 512, N-terminus to C-terminus respectively. The single chain variable fragment (scFv) can comprises a first domain 514 corresponding to a variable light chain domain, or fragment thereof, a second domain 516 corresponding to a variable heavy chain, or a fragment thereof, and a second linker polypeptide 515. The Fab-Fc-scFv:Fc Bispecific IgG structure also comprises a first light chain comprising a VL domain 520 and a CL domain 522. The Fab-Fc-scFv:Fc Bispecific IgG structure also comprises a second light chain comprising a VL domain 524 and a CL domain 526. A heavy chain can be covalently coupled to a light chain molecule via a covalent bond (e.g. disulfide bond 530). A heavy chain can be coupled to another heavy chain via one or more covalent bonds (e.g. disulfide bond 534 and/or 536). The Fab-Fc-scFv:Fc Bispecific IgG structure can comprise a first and a modified second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance) or biochemically (e.g. electrostatic interactions) prevent coupling of the two heavy chain molecules or two second heavy chain molecules. Exemplary mutations that facilitate coupling of a first and a second heavy chain molecule are disclosed, for example in US PG-PUB: US20140322756 and “The making of bispecific antibodies” MAbs. 2017 February-March; 9 (2): 182-212. The Fab-Fc-scFv:Fc Bispecific IgG structure can also comprise carbohydrate molecules 540 coupled thereto or additional modifications thereof.

A bispecific antibody having a Fab-Fc-scFv:Fc Bispecific IgG structure can target a B-cell lineage surface marker (e.g. CD19, CD138, IgA, or CD45e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc-scFv:Fc Bispecific IgG structure can be engineered so that a first antigen binding site targets CD19 and a second antigen binding site targets CD38. In some embodiments, the first heavy chain VH domain (e.g. 502) and VL domain (e.g. 520) comprises a CD19 binding component, wherein the single chain variable fragment (scFv) (e.g. 512) sequence comprises a CD38 binding component. In certain embodiments, the single chain variable fragment (scFv) sequence comprising a CD38 binding component comprises a CD38 binding component corresponding to an antibody heavy chain and light variable sequence, or CD38 binding fragments thereof.

The Fab-Fc-scFv:Fc Bispecific IgG structure can also be engineered so that a first antigen binding site targets CD38 and a second antigen binding site targets CD19. In some embodiments, the first heavy chain VH domain (e.g. 502) and VL domain (e.g. 520) comprises a CD38 binding component, wherein the single chain variable fragment (scFv) (e.g. 512) sequence comprises a CD19 binding component. In certain embodiments, the single chain variable fragment (scFv) sequence comprising a CD19 binding component comprises a CD19 binding component corresponding to an antibody heavy chain and light variable sequence, or CD19 binding fragments thereof.

### Fab-Fc-Fab:Fab-Fc Bispecific IgG

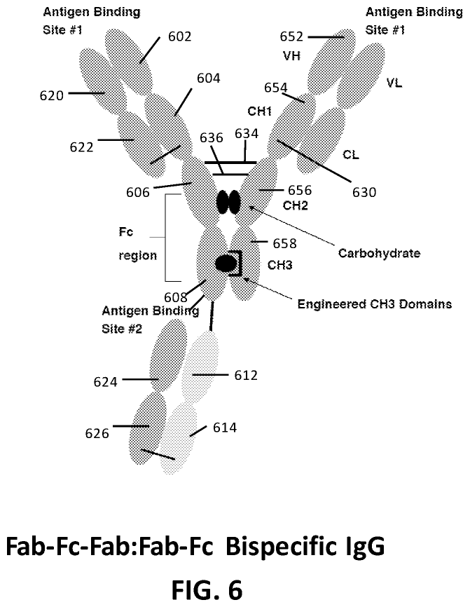

An engineered bispecific antibody having a Fab-Fc-Fab:Fab-Fc Bispecific IgG structure can be used herein. FIG. 6 illustrates a bispecific antibody having a Fab-Fc-Fab:Fab-Fc Bispecific IgG structure. The structure comprises a first heavy chain molecule and a second IgG heavy chain molecule. The first heavy chain comprises VH domain 602, CH1 domain 604, CH2 domain 606, CH3 domain 608, a linker 610 a second VH domain 612, and a second CH1 domain 614, N-terminus to C-terminus respectively. The second heavy chain comprises a VH domain 652, a CH1 domain 654, a CH2 domain 656, and CH3 domain 658, N-terminus to C-terminus respectively, as in that of the first heavy chain. The Fab-Fc-Fab:Fab-Fc Bispecific IgG structure also comprises a first light chain comprising a VL domain 620 and a CL domain 622. The Fab-Fc-Fab:Fab-Fc Bispecific IgG structure also comprises a second light chain comprising a VL domain 624 and a CL domain 626. A heavy chain can be covalently coupled to a light chain molecule via a covalent bond (e.g. disulfide bond 630). The first heavy chain and first light chain can be coupled in a manner that the VH domain and CH1 domain of the first heavy chain pair with the VL domain and CL domain of the first light chain. The first heavy chain and second light chain can be coupled in a manner that the second VH domain and second CH1 domain of the first heavy chain pair with the VL domain and CL domain of the second light chain. A heavy chain can be coupled to another heavy chain via one or more covalent bonds (e.g. disulfide bond 634 and/or 636). The Fab-Fc-Fab:Fab-Fc Bispecific IgG structure can comprise a first and a second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance) or biochemically (e.g. electrostatic interactions) prevent coupling of the two first heavy chain molecules or two second heavy chain molecules. Exemplary mutations that facilitate coupling of a first and a second heavy chain molecule are disclosed, for example in US PG-PUB: US20140322756 and “The making of bispecific antibodies” MAbs. 2017 February-March; 9 (2): 182-212. The Fab-Fc-Fab:Fab-Fc Bispecific IgG structure can also comprise carbohydrate molecules coupled thereto or additional modifications thereof.

A bispecific antibody having a Fab-Fc-Fab:Fab-Fc Bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc-Fab:Fab-Fc Bispecific IgG structure can be engineered so that a first antigen binding site targets CD19 and a second antigen binding site targets CD38. In some embodiments, the first heavy chain VH domain (e.g. 602) and VL domain (e.g. 620) comprises a CD19 binding component, wherein the second VH domain (e.g. 612) and VL domain (e.g. 624) comprises a CD38 binding component.

The Fab-Fc-Fab:Fab-Fc Bispecific IgG structure can also be engineered so that a first antigen binding site targets CD38 and a second antigen binding site targets CD19. In some embodiments, the first heavy chain VH domain (e.g. 602) and VL domain (e.g. 620) comprises a CD38 binding component, wherein the second VH domain (e.g. 612) and VL domain (e.g. 624) comprises a CD19 binding component.

sc Fv-Fab-Fc:scFv-Fab-Fc Bispecific IgG

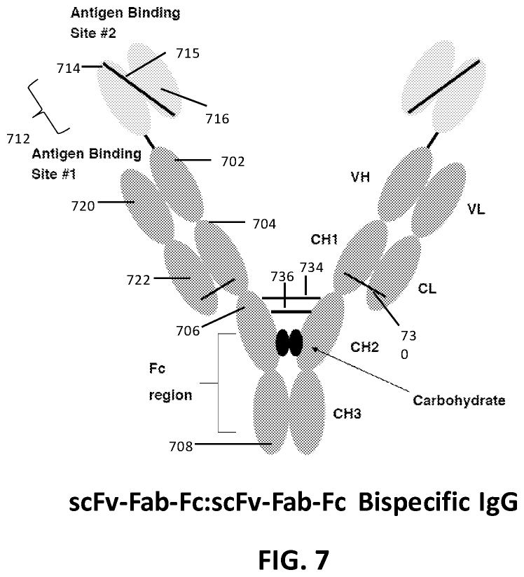

An engineered bispecific antibody having an scFv-Fab-Fc:scFv-Fab-Fc Bispecific IgG structure can be used herein. FIG. 7 illustrates a bispecific antibody having an scFv-Fab-Fc:scFv-Fab-Fc Bispecific IgG structure. The structure comprises a two first heavy chain molecules. The first heavy chain comprises a single chain variable fragment (scFv) 712, a linker 710, VH domain 702, CH1 domain 704, CH2 domain 706, and a CH3 domain 708, N-terminus to C-terminus respectively. The single chain variable fragment (scFv) can comprises a first domain 714 corresponding to a variable light chain domain, or fragment thereof, a second domain 716 corresponding to a variable heavy chain, or a fragment thereof, and a second linker polypeptide 715. The ScFv-Fab-Fc:scFv-Fab-Fc Bispecific IgG structure also comprises a first light chain comprising a VL domain 720 and a CL domain 722. A heavy chain can be covalently coupled to a light chain molecule via a covalent bond (e.g. disulfide bond 730). A heavy chain can be coupled to another heavy chain via one or more covalent bonds (e.g. disulfide bond 734 and/or 736). The ScFv-Fab-Fc:scFv-Fab-Fc Bispecific IgG structure can also comprise carbohydrate molecules 740 coupled thereto or additional modifications thereof.

A bispecific antibody having an scFv-Fab-Fc:scFv-Fab-Fc Bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The scFv-Fab-Fc:scFv-Fab-Fc Bispecific IgG structure can be engineered so that a first antigen binding site targets CD19 and a second antigen binding site targets CD38. In some embodiments, the first heavy chain VH domain (e.g. 702) and VL domain (e.g. 720) comprises a CD19 binding component, wherein the single chain variable fragment (scFv) (e.g. 712) sequence comprises a CD38 binding component. In certain embodiments, the single chain variable fragment (scFv) sequence comprising a CD38 binding component comprises a CD38 binding component corresponding to an antibody heavy chain and light variable sequence, or CD38 binding fragments thereof.

The scFv-Fab-Fc:scFv-Fab-Fc Bispecific IgG structure can also be engineered so that a first antigen binding site targets CD38 and a second antigen binding site targets CD19. In some embodiments, the first heavy chain VH domain (e.g. 702) and VL domain (e.g. 720) comprises a CD38 binding component, wherein the single chain variable fragment (scFv) (e.g. 712) sequence comprises a CD19 binding component. In certain embodiments, the single chain variable fragment (scFv) sequence comprising a CD19 binding component comprises a CD19 binding component corresponding to an antibody heavy chain and light variable sequence, or CD19 binding fragments thereof.

### Fab-Fab-Fc:Fab-Fab-Fc Bispecific IgG

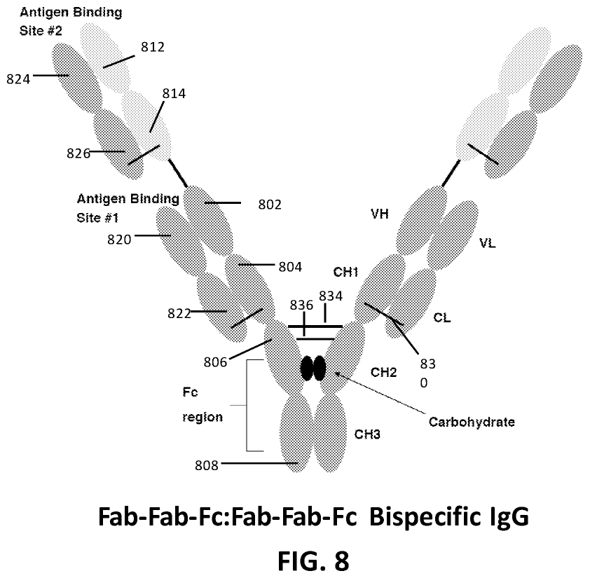

An engineered bispecific antibody having a Fab-Fab-Fc:Fab-Fab-Fc Bispecific IgG structure can be used herein. FIG. 8 illustrates a bispecific antibody having a Fab-Fab-Fc:Fab-Fab-Fc Bispecific IgG structure. The structure comprises two heavy chain molecules. The heavy chain comprises an additional VH domain 812, and an additional CH1 domain 814, a linker 810, VH domain 802, CH1 domain 804, CH2 domain 806, and a CH3 domain 808, N-terminus to C-terminus respectively. The Fab-Fab-Fc:Fab-Fab-Fc Bispecific IgG structure also comprises a first light chain comprising a VL domain 820 and a CL domain 822. The Fab-Fab-Fc:Fab-Fab-Fc Bispecific IgG structure also comprises a second light chain comprising a VL domain 824 and a CL domain 826. A heavy chain molecule can be covalently coupled to a light chain molecule via a covalent bond (e.g. disulfide bond 830). The heavy chain and first light chain can be coupled in a manner that the VH domain and CH1 domain of the heavy chain pair with the VL domain and CL domain of the first light chain. The heavy chain and second light chain can be coupled in a manner that the additional VH domain and additional CH1 domain of the heavy chain pair with the VL domain and CL domain of the second light chain. A heavy chain can be coupled to the modified second heavy chain via one or more covalent bonds (e.g. disulfide bond 834 and/or 836). The Fab-Fab-Fc:Fab-Fab-Fc Bispecific IgG structure can also comprise carbohydrate molecules 840 coupled thereto or additional modifications thereof.

A bispecific antibody having a Fab-Fab-Fc:Fab-Fab-Fc Bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fab-Fc:Fab-Fab-Fc Bispecific IgG structure can be engineered so that a first antigen binding site targets CD19 and a second antigen binding site targets CD38. In some embodiments, the first VH domain (e.g. 802) and VL domain (e.g. 820) comprise a CD19 binding component, wherein the second VH domain (e.g. 812) and VL domain (e.g. 824) comprises a CD38 binding component.

The Fab-Fab-Fc:Fab-Fab-Fc Bispecific IgG structure can also be engineered so that a first antigen binding site targets CD38 and a second antigen binding site targets CD19. In some embodiments, the VH domain (e.g. 802) and VL domain (e.g. 820) comprises a CD38 binding component, wherein the second VH domain (e.g. 812) and VL domain (e.g. 824) comprises a CD19 binding component.

### Fab-Fc-Fab:Fab-Fc-Fab Bispecific IgG

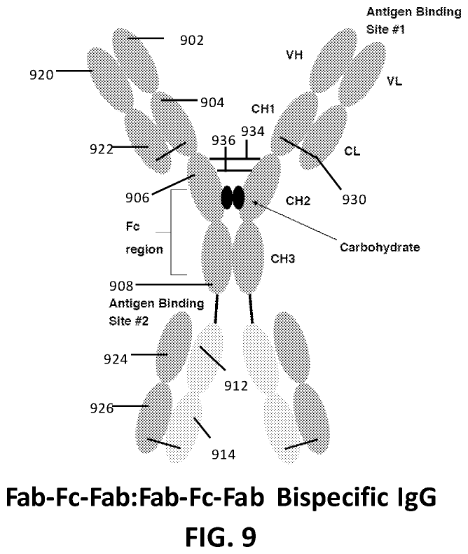

An engineered bispecific antibody having a Fab-Fc-Fab:Fab-Fc-Fab Bispecific IgG structure can be used herein. FIG. 9 illustrates a bispecific antibody having a Fab-Fc-Fab:Fab-Fc-Fab Bispecific IgG structure. The structure comprises two heavy chain molecules and two light chain molecules. The heavy chain comprises VH domain 902, CH1 domain 904, CH2 domain 906, CH3 domain 908, a linker 910 a second VH domain 912, and a second CH1 domain 914, N-terminus to C-terminus respectively. The Fab-Fc-Fab:Fab-Fc-Fab Bispecific IgG structure also comprises a first light chain comprising a VL domain 920 and a CL domain 922. The Fab-Fc-Fab:Fab-Fc-Fab Bispecific IgG structure also comprises a second light chain comprising a VL domain 924 and a CL domain 926. A heavy chain can be covalently coupled to a light chain molecule via a covalent bond (e.g. disulfide bond 930). The heavy chain and first light chain can be coupled in a manner that the VH domain and CH1 domain of the heavy chain pair with the VL domain and CL domain of the first light chain. The heavy chain and second light chain can be coupled in a manner that the second VH domain and second CH1 domain of the heavy chain pair with the VL domain and CL domain of the second light chain. A heavy chain can also be covalently coupled to another heavy chain molecule via a covalent bond (e.g. disulfide bond 934 and 936). The Fab-Fc-Fab Bispecific IgG structure can also comprise carbohydrate molecules 940 coupled thereto or additional modifications thereof.

A bispecific antibody having a Fab-Fc-Fab:Fab-Fc-Fab Bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc-Fab:Fab-Fc-Fab Bispecific IgG structure can be engineered so that a first antigen binding site targets CD19 and a second antigen binding site targets CD38. In some embodiments, the first VH domain (e.g. 902) and VL domain (e.g. 920) comprise a CD19 binding component, wherein the second VH domain (e.g. 912) and VL domain (e.g. 924) comprises a CD38 binding component.

The Fab-Fc-Fab:Fab-Fc-Fab Bispecific IgG structure can also be engineered so that a first antigen binding site targets CD38 and a second antigen binding site targets CD19. In some embodiments, the VH domain (e.g. 902) and VL domain (e.g. 920) comprises a CD38 binding component, wherein the second VH domain (e.g. 912) and VL domain (e.g. 924) comprises a CD19 binding component.

### Fab-Fc-scFv:Fab-Fc Bispecific IgG

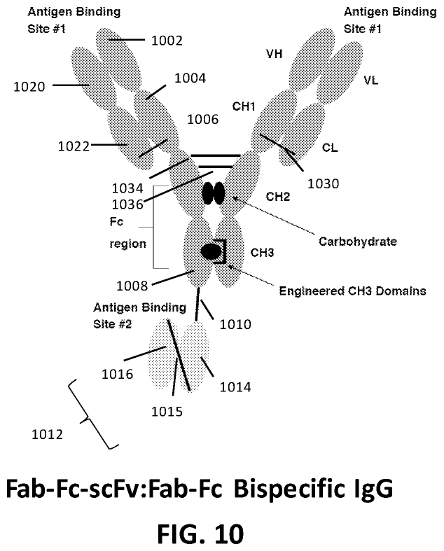

An engineered bispecific antibody having a Fab-Fc-scFv:Fab-Fc Bispecific IgG structure can be used herein. FIG. 10 demonstrates a bispecific antibody having a Fab-Fc-scFv:Fab-Fc Bispecific IgG structure. The structure comprises a first heavy chain molecule and a second IgG heavy chain molecule. The first heavy chain comprises VH domain 1002, CH1 domain 1004, CH2 domain 1006, CH3 domain 1008, a linker 1010 and a single chain variable fragment (scFv) 1012, N-terminus to C-terminus respectively. The single chain variable fragment (scFv) can comprises a first domain 1014 corresponding to a variable light chain domain, or fragment thereof, a second domain 1016 corresponding to a variable heavy chain, or a fragment thereof, and a second linker polypeptide 1015. The second heavy chain comprises a VH domain 1002, a CH1 domain 1004, a CH2 domain 1004, and CH3 domain 1008, N-terminus to C-terminus respectively, as in that of the first heavy chain. The Fab-Fc-scFv:Fab-Fc Bispecific IgG structure also comprises a first light chain comprising a VL domain 1020 and a CL domain 1022. A heavy chain can be covalently coupled to a light chain molecule via a covalent bond (e.g. disulfide bond 1030). A heavy chain can be coupled to another heavy chain via one or more covalent bonds (e.g. disulfide bond 1034 and/or 1036). The Fab-Fc-scFv:Fab-Fc Bispecific IgG structure can comprise a first and a second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. steric hinderance) or biochemically (e.g. electrostatic interactions) prevent coupling of the two first heavy chain molecules or two second heavy chain molecules. Exemplary mutations that facilitate coupling of a first and a second heavy chain molecule are disclosed, for example in US PG-PUB: US20140322756 and “The making of bispecific antibodies” MAbs. 2017 February-March; 9 (2): 182-212. The Fab-Fc-scFv:Fab-Fc Bispecific IgG structure can also comprise carbohydrate molecules 1040 coupled thereto or additional modifications thereof.

A bispecific antibody having a Fab-Fc-scFv:Fab-Fc Bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The Fab-Fc-scFv:Fab-Fc Bispecific IgG structure can be engineered so that a first antigen binding site targets CD19 and a second antigen binding site targets CD38. In some embodiments, the first heavy chain VH domain (e.g. 1002) and VL domain (e.g. 1020) comprises a CD19 binding component, wherein the single chain variable fragment (scFv) (e.g. 1012) sequence comprises a CD38 binding component. In certain embodiments, the single chain variable fragment (scFv) sequence comprising a CD38 binding component comprises a CD38 binding component corresponding to an antibody heavy chain and light variable sequence, or CD38 binding fragments thereof.

The Fab-Fc-scFv:Fab-Fc Bispecific IgG structure can also be engineered so that a first antigen binding site targets CD38 and a second antigen binding site targets CD19. In some embodiments, the first heavy chain VH domain (e.g. 1002) and VL domain (e.g. 1020) comprises a CD38 binding component, wherein the single chain variable fragment (scFv) (e.g. 1012) sequence comprises a CD19 binding component. In certain embodiments, the single chain variable fragment (scFv) sequence comprising a CD19 binding component comprises a CD19 binding component corresponding to an antibody heavy chain and light variable sequence, or CD19 binding fragments thereof.

scFv-Fab-Fc:Fc Bispecific IgG

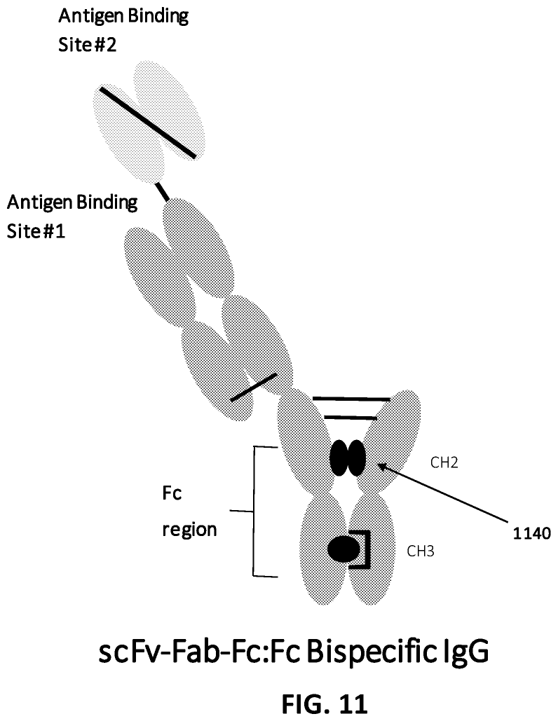

An engineered bispecific antibody having a scFv-Fab-Fc:Fc Bispecific IgG structure can be used herein. FIG. 11 demonstrates a bispecific antibody having a scFv-Fab-Fc:Fc Bispecific IgG structure. The structure comprises a first heavy chain molecule comprising an scFv, VH, and an Fc region and a second heavy chain molecule comprising an Fc. The scFv-Fab-Fc:Fc Bispecific IgG structure can comprise a first and a second heavy chain molecule that further comprises mutations within the CH3 domain that promote coupling of the first and the second heavy chain and/or prevent coupling of a first heavy chain to another first heavy chain or a second heavy chain to another second heavy chain. The mutations can physically (e.g. Knob-in hole architecture) or biochemically (e.g. electrostatic interactions) promote association of the first heavy chain molecule to the second heavy chain molecule. The scFv-Fab-Fc:Fc Bispecific IgG structure comprises a light chain molecule associated with the first heavy chain molecule that creates a first antigen binding site. A second antigen binding site is provided by an scFv fragment coupled to the N-terminal endo of the first heavy chain. Exemplary mutations that facilitate coupling of a first and a second heavy chain molecule are disclosed, for example in US PG-PUB: US20140322756 and “The making of bispecific antibodies” MAbs. 2017 February-March; 9 (2): 182-212. The scFv-Fab-Fc:Fc Bispecific IgG structure can also comprise carbohydrate molecules 1140 coupled thereto or additional modifications thereof.

A bispecific antibody having an scFv-Fab-Fc:Fc Bispecific IgG structure can target a B-cell lineage surface marker (e.g. CD19, CD138, IgA, or CD45), and a suppressive B-cell surface marker (e.g. IgD, CD1, CD5, CD21, CD24, CD38, HM13, SLAMF7, AQP3, or latent TGF-beta (e.g., TGF-beta LAP)). In some embodiments, the B-cell lineage surface marker comprises CD19. In certain embodiments, the B-cell lineage surface marker consists of CD19. In some embodiments, the suppressive B-cell surface marker comprises CD38. In certain embodiments, the suppressive B-cell surface marker consists of CD38.

The scFv-Fab-Fc:Fc Bispecific IgG structure can be engineered so that a first antigen binding site targets CD19 and a second antigen binding site targets CD38. In some embodiments, the first heavy chain VH domain and VL domain comprises a CD19 binding component, wherein the single chain variable fragment (scFv) sequence comprises a CD38 binding component. In certain embodiments, the single chain variable fragment (scFv) sequence comprises a CD38 binding component corresponding to an antibody heavy chain and light variable sequence, or CD38 binding fragments thereof.

The scFv-Fab-Fc:Fc Bispecific IgG structure can also be engineered so that a first antigen binding site targets CD38 and a second antigen binding site targets CD19. In some embodiments, the heavy chain VH domain and VL domain comprises a CD38 binding component, wherein the single chain variable fragment (scFv) sequence comprises a CD19 binding component. In certain embodiments, the single chain variable fragment (scFv) sequence comprising a CD19 binding component comprises a CD19 binding component corresponding to an antibody heavy chain and light variable sequence, or CD19 binding fragments thereof.

In certain embodiments, the first heavy chain molecule comprises an amino acid sequence at least about 90%, 95%, 97%, 98%, or 99% identical to the amino acid sequence set forth in SEQ ID NO: 212. In certain embodiments, the first heavy chain molecule comprises an amino acid sequence identical to the amino acid sequence set forth in SEQ ID NO: 212.

In certain embodiments, the light chain molecule comprises an amino acid sequence at least about 90%, 95%, 97%, 98%, or 99% identical to the amino acid sequence set forth in SEQ ID NO: 213. In certain embodiments, the light chain molecule comprises an amino acid sequence identical to the amino acid sequence set forth in SEQ ID NO: 213.

In certain embodiments, the second heavy chain molecule comprises an amino acid sequence at least about 90%, 95%, 97%, 98%, or 99% identical to the amino acid sequence set forth in SEQ ID NO: 214. In certain embodiments, the first heavy chain molecule comprises an amino acid sequence identical to the amino acid sequence set forth in SEQ ID NO: 214.

### Fc Variants

In some embodiments, one or more amino acid modifications are introduced into the Fragment crystallizable (Fc) region of a human or humanized antibody, thereby generating an Fc region variant. An Fc region may comprise a C-terminal region of an immunoglobulin heavy chain that comprises a hinge region, CH2 domain, CH3 domain, or any combination thereof. As used herein, an Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution, addition, or deletion) at one or more amino acid positions.

In some embodiments, a variant Fc region comprises at least one amino acid modification in the Fc region. Combining amino acid modifications are also useful. For example, the variant Fc region may include two, three, four, five, etc. substitutions therein, e.g. of the specific Fc region positions identified herein.

In some embodiments, the antibodies described herein have a reduced effector function as compared to a human IgG. Effector functions generally refer to a biological event resulting from the interaction of an antibody Fc region with an Fc receptor or ligand. Non-limiting effector functions include C1q binding, complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation. In some cases, antibody-dependent cell-mediated cytotoxicity (ADCC) refers to a cell-mediated reaction in which nonspecific cytotoxic cells expressing Fc receptors (e.g., natural killer cells, neutrophils, macrophages) recognize bound antibody on a target cell, subsequently causing lysis of the target cell. In some cases, complement dependent cytotoxicity (CDC) refers to lysing of a target cells in the presence of complement, where the complement action pathway is initiated by the binding of C1q to antibody bound with the target.

In certain cases, it is beneficial to reduce the effector function of the antibodies described herein. In some instances, modifications in the Fc region generate an Fc variant with (a) decreased antibody-dependent cell-mediated cytotoxicity ADCC), (b) decreased complement mediated cytotoxicity (CDC), and/or (c) decreased affinity for C1q. In some embodiments, the Fc region is modified to decrease antibody dependent cellular cytotoxicity (ADCC), decrease antibody-dependent cell-mediated phagocytosis (ADCP), decrease complement mediated cytotoxicity (CDC), and/or decrease affinity for C1q by modifying one or more amino acids at the following positions: 234, 235, 236, 238, 239, 240, 241, 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439 (Kabat numbering). In some embodiments, the variant Fc region is selected from Table 1. In some embodiments, the variant Fc region comprises one or more of the mutations of Table 1.

TABLE 1

| Variant Fc Regions | |
|---|---|
| and Mutation(s) | Exemplary Effect |
| E233P | Decreases binding to FcγRI, II, III |
| S228P, L235E | Decreases binding to FcγRI |
| SPLE in IgG4 | |
| L235E | Decreases binding to FcγRs |
| L234A, L235A | Decreases binding to FcγRI, II, III |
| L234A, L235A, G237A | Decreases binding to FcγRI, II, III, C1q |
| L234A, L235A, P329G | Decreases binding to FcγRI, II, III, C1q |
| L234F, L235E, P331S | Decreases binding to FcγRI, II, III, C1q |
| L234A, L235E, G237A | Decreases binding to FcγRI, II, III, C1q |
| L234A, L235E, | Decreases binding to FcγRI, II, III, C1q |
| G237A, P331S | |
| L234A, L235E, G237A, | Decreases binding to FcγRI, II, III, C1q |
| A330S, P331S | |
| L234A, L235A, | Decreases binding to FcγRI, |
| G237A, P238S, | IIa, IIb, IIIa |
| H268A, A330S, | |
| P331S (IgG1σ) | |
| L234A, L235A, P329A | Decreases binding to FcγRI, II, III, C1q |
| G236R, L328R | Decreases binding to FcγRI, II, III |
| G237A | Decreases binding to FcγRII |
| F241A | Decreases binding to C1q |
| V264A | Decreases binding to C1q |
| D265A | Decreases binding to FcγRI, II, III |
| D265A, N297A | Decreases binding to FcγRI, II, III, C1q |
| D265A, N297G | Decreases binding to FcγRI, II, III, C1q |
| D270A | Decreases binding to C1q |
| N297A, G, D, Q | Elimination of N-linked glycosylation |
| | Decreases binding to FcγRI, II, III, C1q |
| P329A, G, R | Decreases binding to C1q |
| A330L | Decreases binding to C1q |
| P331A or P331S | Diminished C1q binding |
| S228P | Prevent IgG4 Fab arm exchange |
| S228P, F234A, | Decreases binding to FcγRI, IIa, IIIa |
| L235A (IgG4) | |
| H268Q, V309L, A330S, | Decreases binding to FcγRI, II, III, C1q |
| P331S (IgG2m4) | |
| V234A, G237A, P238S, | Decreases binding to FcγRI, IIa, |
| H268A, V309L, A330S, | |
| P331S (IgG2σ) | IIb, IIIa, C1q |
| K322A | Decreases binding to C1q |
| N297A or N297Q | Reduced FcγR and C1q binding |
| or N297G | |
| S239D, I332E | Reduced FcγR and C1q binding |
| H268Q, V309L, | Reduced FcγR and C1q binding |
| A330S, P331S | |
| V234A, G237A, | Reduced FcγR and C1q binding |
| P238S, H268A, | |
| V309L, A330S, P331S | |
| High mannose | Decreases binding to C1q |
| glycosylation | |

Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

In some embodiments, variant Fc regions exhibit ADCC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease ADCC (such as human IgG1). In some embodiments, variant Fc regions exhibit CDC that is reduced by at least about 30%, at least about 40%, at least about 50%, at least about 60%, at least about 70% or more as compared to an antibody comprising a non-variant Fc region, i.e., an antibody with the same sequence identity but for the substitution(s) that decrease CDC (such as human IgG1).

In certain embodiments, variant Fc regions exhibit ADCC that is reduced by about 10% to about 100%. In certain embodiments, variant Fc regions exhibit ADCC that is reduced by about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In certain embodiments, variant Fc regions exhibit ADCC that is reduced by about 10%, about 20%, about 30%, about 40%, about 50%, about 60%, about 70%, about 80%, about 90%, or about 100%. In certain embodiments, variant Fc regions exhibit ADCC that is reduced by at least about 10%, about 20%, about 30%, about 40%, about 50%, about 60%, about 70%, about 80%, or about 90%.

In certain embodiments, variant Fc regions exhibit CDC that is reduced by about 10% to about 100%. In certain embodiments, variant Fc regions exhibit CDC that is reduced by about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In certain embodiments, variant Fc regions exhibit CDC that is reduced by about 10%, about 20%, about 30%, about 40%, about 50%, about 60%, about 70%, about 80%, about 90%, or about 100%. In certain embodiments, variant Fc regions exhibit CDC that is reduced by at least about 10%, about 20%, about 30%, about 40%, about 50%, about 60%, about 70%, about 80%, or about 90%.

In some embodiments, variant Fc regions exhibit reduced effector function as compared with wild-type human IgG1. Non-limiting examples of Fc mutations in IgG1 that, in certain instances, reduce ADCC and/or CDC include substitutions at one or more of positions: 231, 232, 234, 235, 236, 237, 238, 239, 264, 265, 267, 269, 270, 297, 299, 318, 320, 322, 325, 327, 328, 329, 330, and 331 in IgG1, where the numbering system of the constant region is that of the EU index as set forth by Kabat.

In some embodiments, the variant Fc region comprises an IgG1 Fc region comprising an N297A substitution, according to the Kabat numbering system. In some embodiments, the variant Fc region comprises an IgG1 Fc region comprising an N297Q substitution, according to the Kabat numbering system. In some embodiments, the variant Fc region comprises an IgG1 Fc region comprising an N297D substitution, according to the Kabat numbering system. In some embodiments, the variant Fc region comprises an IgG1 Fc region comprising an D265A substitution, according to the Kabat numbering system. In some embodiments, the variant Fc region comprises an IgG1 Fc region comprising an S228P substitution, according to the Kabat numbering system. In some embodiments, the variant Fc region comprises an IgG1 Fc region comprising an L235A substitution, according to the Kabat numbering system. In some embodiments, the variant Fc region comprises an IgG1 Fc region comprising an L237A substitution, according to the Kabat numbering system. In some embodiments, the variant Fc region comprises an IgG1 Fc region comprising an L234A substitution, according to the Kabat numbering system. In some embodiments, the variant Fc region comprises an IgG1 Fc region comprising an E233P substitution, according to the Kabat numbering system. In some embodiments, the variant Fc region comprises an IgG1 Fc region comprising an L234V substitution, according to the Kabat numbering system. In some embodiments, the variant Fc region comprises an IgG1 Fc region comprising an C236 deletion, according to the Kabat numbering system. In some embodiments, the variant Fc region comprises an IgG1 Fc region comprising a P238A substitution, according to the Kabat numbering system. In some embodiments, the variant Fc region comprises an IgG1 Fc region comprising an A327Q substitution, according to the Kabat numbering system. In some embodiments, the variant Fc region comprises an IgG1 Fc region comprising a P329A substitution, according to the Kabat numbering system. In some embodiments, the variant Fc region comprises an IgG1 Fc region comprising an P329G substitution, according to the Kabat numbering system. In some embodiments, the variant Fc region comprises an IgG1 Fc region comprising an L235E substitution, according to the Kabat numbering system. In some embodiments, the variant Fc region comprises an IgG1 Fc region comprising an P331S substitution, according to the Kabat numbering system. In some embodiments, the variant Fc region comprises an IgG1 Fc region comprising an L234F substitution, according to the Kabat numbering system. In some embodiments, the variant Fc region comprises an IgG1 Fc region comprising a 235G substitution, according to the Kabat numbering system. In some embodiments, the variant Fc region comprises an IgG1 Fc region comprising an 235Q substitution, according to the Kabat numbering system. In some embodiments, the variant Fc region comprises an IgG1 Fc region comprising an 235R substitution, according to the Kabat numbering system. In some embodiments, the variant Fc region comprises an IgG1 Fc region comprising an 235S substitution, according to the Kabat numbering system. In some embodiments, the variant Fc region comprises an IgG1 Fc region comprising an 236F substitution, according to the Kabat numbering system. In some embodiments, the variant Fc region comprises an IgG1 Fc region comprising an 236R substitution, according to the Kabat numbering system. In some embodiments, the variant Fc region comprises an IgG1 Fc region comprising an 237E substitution, according to the Kabat numbering system. In some embodiments, the variant Fc region comprises an IgG1 Fc region comprising an 237K substitution, according to the Kabat numbering system. In some embodiments, the variant Fc region comprises an IgG1 Fc region comprising an 237N substitution, according to the Kabat numbering system. In some embodiments, the variant Fc region comprises an IgG1 Fc region comprising an 237R substitution, according to the Kabat numbering system. In some embodiments, the variant Fc region comprises an IgG1 Fc region comprising an 238A substitution, according to the Kabat numbering system. In some embodiments, the variant Fc region comprises an IgG1 Fc region comprising an 238E substitution, according to the Kabat numbering system. In some embodiments, the variant Fc region comprises an IgG1 Fc region comprising an 238G substitution, according to the Kabat numbering system. In some embodiments, the variant Fc region comprises an IgG1 Fc region comprising an 238H substitution, according to the Kabat numbering system. In some embodiments, the variant Fc region comprises an IgG1 Fc region comprising an 238I substitution, according to the Kabat numbering system. In some embodiments, the variant Fc region comprises an IgG1 Fc region comprising an 238V substitution, according to the Kabat numbering system. In some embodiments, the variant Fc region comprises an IgG1 Fc region comprising an 238W substitution, according to the Kabat numbering system. In some embodiments, the variant Fc region comprises an IgG1 Fc region comprising an 238Y substitution, according to the Kabat numbering system. In some embodiments, the variant Fc region comprises an IgG1 Fc region comprising an 248A substitution, according to the Kabat numbering system. In some embodiments, the variant Fc region comprises an IgG1 Fc region comprising an 254D substitution, according to the Kabat numbering system. In some embodiments, the variant Fc region comprises an IgG1 Fc region comprising an 254E substitution, according to the Kabat numbering system. In some embodiments, the variant Fc region comprises an IgG1 Fc region comprising an 254G substitution, according to the Kabat numbering system. In some embodiments, the variant Fc region comprises an IgG1 Fc region comprising an 254H substitution, according to the Kabat numbering system. In some embodiments, the variant Fc region comprises an IgG1 Fc region comprising an 254I substitution, according to the Kabat numbering system. In some embodiments, the variant Fc region comprises an IgG1 Fc region comprising an 254N substitution, according to the Kabat numbering system. In some embodiments, the variant Fc region comprises an IgG1 Fc region comprising an 254P substitution, according to the Kabat numbering system. In some embodiments, the variant Fc region comprises an IgG1 Fc region comprising an 254Q substitution, according to the Kabat numbering system. In some embodiments, the variant Fc region comprises an IgG1 Fc region comprising an 254T substitution, according to the Kabat numbering system. In some embodiments, the variant Fc region comprises an IgG1 Fc region comprising an 254V substitution, according to the Kabat numbering system. In some embodiments, the variant Fc region comprises an IgG1 Fc region comprising an 255N substitution, according to the Kabat numbering system. In some embodiments, the variant Fc region comprises an IgG1 Fc region comprising an 256H substitution, according to the Kabat numbering system. In some embodiments, the variant Fc region comprises an IgG1 Fc region comprising an 256K substitution, according to the Kabat numbering system. In some embodiments, the variant Fc region comprises an IgG1 Fc region comprising an 256R substitution, according to the Kabat numbering system. In some embodiments, the variant Fc region comprises an IgG1 Fc region comprising an 256V substitution, according to the Kabat numbering system. In some embodiments, the variant Fc region comprises an IgG1 Fc region comprising an 264S substitution, according to the Kabat numbering system. In some embodiments, the variant Fc region comprises an IgG1 Fc region comprising an 265H substitution, according to the Kabat numbering system. In some embodiments, the variant Fc region comprises an IgG1 Fc region comprising an 265K substitution, according to the Kabat numbering system. In some embodiments, the variant Fc region comprises an IgG1 Fc region comprising an 265S substitution, according to the Kabat numbering system. In some embodiments, the variant Fc region comprises an IgG1 Fc region comprising an 265Y substitution, according to the Kabat numbering system. In some embodiments, the variant Fc region comprises an IgG1 Fc region comprising an 267G substitution, according to the Kabat numbering system. In some embodiments, the variant Fc region comprises an IgG1 Fc region comprising an 267H substitution, according to the Kabat numbering system. In some embodiments, the variant Fc region comprises an IgG1 Fc region comprising an 267I substitution, according to the Kabat numbering system. In some embodiments, the variant Fc region comprises an IgG1 Fc region comprising an 267K substitution, according to the Kabat numbering system. In some embodiments, the variant Fc region comprises an IgG1 Fc region comprising an 268K substitution, according to the Kabat numbering system. In some embodiments, the variant Fc region comprises an IgG1 Fc region comprising an 269N substitution, according to the Kabat numbering system. In some embodiments, the variant Fc region comprises an IgG1 Fc region comprising an 269Q substitution, according to the Kabat numbering system. In some embodiments, the variant Fc region comprises an IgG1 Fc region comprising an 270A substitution, according to the Kabat numbering system. In some embodiments, the variant Fc region comprises an IgG1 Fc region comprising an 270G substitution, according to the Kabat numbering system. In some embodiments, the variant Fc region comprises an IgG1 Fc region comprising an 270M substitution, according to the Kabat numbering system. In some embodiments, the variant Fc region comprises an IgG1 Fc region comprising an 270N substitution, according to the Kabat numbering system. In some embodiments, the variant Fc region comprises an IgG1 Fc region comprising an 271T substitution, according to the Kabat numbering system. In some embodiments, the variant Fc region comprises an IgG1 Fc region comprising an 272N substitution, according to the Kabat numbering system. In some embodiments, the variant Fc region comprises an IgG1 Fc region comprising an 279F substitution, according to the Kabat numbering system. In some embodiments, the variant Fc region comprises an IgG1 Fc region comprising an 279K substitution, according to the Kabat numbering system. In some embodiments, the variant Fc region comprises an IgG1 Fc region comprising an 279L substitution, according to the Kabat numbering system. In some embodiments, the variant Fc region comprises an IgG1 Fc region comprising an 292E substitution, according to the Kabat numbering system. In some embodiments, the variant Fc region comprises an IgG1 Fc region comprising an 292F substitution, according to the Kabat numbering system. In some embodiments, the variant Fc region comprises an IgG1 Fc region comprising an 292G substitution, according to the Kabat numbering system. In some embodiments, the variant Fc region comprises an IgG1 Fc region comprising an 292I substitution, according to the Kabat numbering system. In some embodiments, the variant Fc region comprises an IgG1 Fc region comprising an 293S substitution, according to the Kabat numbering system. In some embodiments, the variant Fc region comprises an IgG1 Fc region comprising an 301W substitution, according to the Kabat numbering system. In some embodiments, the variant Fc region comprises an IgG1 Fc region comprising an 304E substitution, according to the Kabat numbering system. In some embodiments, the variant Fc region comprises an IgG1 Fc region comprising an 311E substitution, according to the Kabat numbering system. In some embodiments, the variant Fc region comprises an IgG1 Fc region comprising an 311G substitution, according to the Kabat numbering system. In some embodiments, the variant Fc region comprises an IgG1 Fc region comprising an 311S substitution, according to the Kabat numbering system. In some embodiments, the variant Fc region comprises an IgG1 Fc region comprising an 316F substitution, according to the Kabat numbering system. In some embodiments, the variant Fc region comprises an IgG1 Fc region comprising an 327T substitution, according to the Kabat numbering system. In some embodiments, the variant Fc region comprises an IgG1 Fc region comprising an 328V substitution, according to the Kabat numbering system. In some embodiments, the variant Fc region comprises an IgG1 Fc region comprising an 329Y substitution, according to the Kabat numbering system. In some embodiments, the variant Fc region comprises an IgG1 Fc region comprising an 330R substitution, according to the Kabat numbering system. In some embodiments, the variant Fc region comprises an IgG1 Fc region comprising an 339E substitution, according to the Kabat numbering system. In some embodiments, the variant Fc region comprises an IgG1 Fc region comprising an 339L substitution, according to the Kabat numbering system. In some embodiments, the variant Fc region comprises an IgG1 Fc region comprising an 343I substitution, according to the Kabat numbering system. In some embodiments, the variant Fc region comprises an IgG1 Fc region comprising an 343V substitution, according to the Kabat numbering system. In some embodiments, the variant Fc region comprises an IgG1 Fc region comprising an 373A substitution, according to the Kabat numbering system. In some embodiments, the variant Fc region comprises an IgG1 Fc region comprising an 373G substitution, according to the Kabat numbering system. In some embodiments, the variant Fc region comprises an IgG1 Fc region comprising an 373 S substitution, according to the Kabat numbering system. In some embodiments, the variant Fc region comprises an IgG1 Fc region comprising an 376E substitution, according to the Kabat numbering system. In some embodiments, the variant Fc region comprises an IgG1 Fc region comprising an 376W substitution, according to the Kabat numbering system. In some embodiments, the variant Fc region comprises an IgG1 Fc region comprising an 376Y substitution, according to the Kabat numbering system. In some embodiments, the variant Fc region comprises an IgG1 Fc region comprising an 380D substitution, according to the Kabat numbering system. In some embodiments, the variant Fc region comprises an IgG1 Fc region comprising an 382D substitution, according to the Kabat numbering system. In some embodiments, the variant Fc region comprises an IgG1 Fc region comprising an 382P substitution, according to the Kabat numbering system. In some embodiments, the variant Fc region comprises an IgG1 Fc region comprising an 385P substitution, according to the Kabat numbering system. In some embodiments, the variant Fc region comprises an IgG1 Fc region comprising an 424H substitution, according to the Kabat numbering system. In some embodiments, the variant Fc region comprises an IgG1 Fc region comprising an 424M substitution, according to the Kabat numbering system. In some embodiments, the variant Fc region comprises an IgG1 Fc region comprising an 424V substitution, according to the Kabat numbering system. In some embodiments, the variant Fc region comprises an IgG1 Fc region comprising an 434I substitution, according to the Kabat numbering system. In some embodiments, the variant Fc region comprises an IgG1 Fc region comprising an 438G substitution, according to the Kabat numbering system. In some embodiments, the variant Fc region comprises an IgG1 Fc region comprising an 439E substitution, according to the Kabat numbering system. In some embodiments, the variant Fc region comprises an IgG1 Fc region comprising an 439H substitution, according to the Kabat numbering system. In some embodiments, the variant Fc region comprises an IgG1 Fc region comprising an 439Q substitution, according to the Kabat numbering system. In some embodiments, the variant Fc region comprises an IgG1 Fc region comprising an 440A substitution, according to the Kabat numbering system. In some embodiments, the variant Fc region comprises an IgG1 Fc region comprising an 440D substitution, according to the Kabat numbering system. In some embodiments, the variant Fc region comprises an IgG1 Fc region comprising an 440E substitution, according to the Kabat numbering system. In some embodiments, the variant Fc region comprises an IgG1 Fc region comprising an 440F substitution, according to the Kabat numbering system. In some embodiments, the variant Fc region comprises an IgG1 Fc region comprising an 440M substitution, according to the Kabat numbering system. In some embodiments, the variant Fc region comprises an IgG1 Fc region comprising an 440T Fc region substitution, according to the Kabat numbering system. In some embodiments, the variant Fc region comprises an IgG1 Fc region comprising an 440V substitution, according to the Kabat numbering system.

In some embodiments, the variant Fc region comprises an IgG1 Fc region L234A, L235E, G237A, A330S, and/or P331S by Kabat Numbering. In some embodiments, the variant Fc region comprises an IgG1 Fc region comprising E233P, according to the Kabat numbering system. In some embodiments, the variant Fc region comprises an IgG4 Fc region comprising S228P and L235E. In some embodiments, the variant Fc region comprises an IgG1 Fc region comprising L235E, according to the Kabat numbering system. In some embodiments, the variant Fc region comprises an IgG1 Fc region comprising L234A and L235A, according to the Kabat numbering system. In some embodiments, the variant Fc region comprises an IgG1 Fc region comprising L234A, L235A, and G237A, according to the Kabat numbering system. In some embodiments, the variant Fc region comprises an IgG1 Fc region comprising L234A, L235A, P329G, according to the Kabat numbering system. In some embodiments, the variant Fc region comprises an IgG1 Fc region comprising L234F, L235E, and P33 IS, according to the Kabat numbering system. In some embodiments, the variant Fc region comprises an IgG1 Fc region comprising L234A, L235E, and G237A, according to the Kabat numbering system. In some embodiments, the variant Fc region comprises an IgG1 Fc region comprising L234A, L235E, G237A, and P331S, according to the Kabat numbering system. In some embodiments, the variant Fc region comprises an IgG1 Fc region comprising L234A, L235A, G237A, P238S, H268A, A330S, and P331S (IgG1), according to the Kabat numbering system. In some embodiments, the variant Fc region comprises an IgG1 Fc region comprising L234A, L235A, and P329A, according to the Kabat numbering system. In some embodiments, the variant Fc region comprises an IgG1 Fc region comprising G236R and L328R, according to the Kabat numbering system. In some embodiments, the variant Fc region comprises an IgG1 Fc region comprising G237A, according to the Kabat numbering system. In some embodiments, the variant Fc region comprises an IgG1 Fc region comprising F241A, according to the Kabat numbering system. In some embodiments, the variant Fc region comprises an IgG1 Fc region comprising V264A, according to the Kabat numbering system. In some embodiments, the variant Fc region comprises an IgG1 Fc region comprising D265A, according to the Kabat numbering system. In some embodiments, the variant Fc region comprises an IgG1 Fc region comprising D265A and N297A, according to the Kabat numbering system. In some embodiments, the variant Fc region comprises an IgG1 Fc region comprising D265A and N297G, according to the Kabat numbering system. In some embodiments, the variant Fc region comprises an IgG1 Fc region comprising D270A, according to the Kabat numbering system. In some embodiments, the variant Fc region comprises an IgG1 Fc region comprising N297A, according to the Kabat numbering system. In some embodiments, the variant Fc region comprises an IgG1 Fc region comprising N297G, according to the Kabat numbering system. In some embodiments, the variant Fc region comprises an IgG1 Fc region comprising N297D, according to the Kabat numbering system. In some embodiments, the variant Fc region comprises an IgG1 Fc region comprising N297Q, according to the Kabat numbering system. In some embodiments, the variant Fc region comprises an IgG1 Fc region comprising P329A, according to the Kabat numbering system. In some embodiments, the variant Fc region comprises an IgG1 Fc region comprising P329G, according to the Kabat numbering system. In some embodiments, the variant Fc region comprises an IgG1 Fc region comprising P329R, according to the Kabat numbering system. In some embodiments, the variant Fc region comprises an IgG1 Fc region comprising A330L, according to the Kabat numbering system. In some embodiments, the variant Fc region comprises an IgG1 Fc region comprising P331A, according to the Kabat numbering system. In some embodiments, the variant Fc region comprises an IgG1 Fc region comprising P331S, according to the Kabat numbering system. In some embodiments, the variant Fc region comprises an IgG2 Fc region. In some embodiments, the variant Fc region comprises an IgG4 Fc region. In some embodiments, the variant Fc region comprises an IgG4 Fc region comprising S228P, according to the Kabat numbering system. In some embodiments, the variant Fc region comprises an IgG4 Fc region comprising S228P, F234A, and L235A, according to the Kabat numbering system. In some embodiments, the variant Fc region comprises an IgG2-IgG4 cross-subclass (IgG2/G4) Fc region. In some embodiments, the variant Fc region comprises an IgG2-IgG3 cross-subclass Fc region. In some embodiments, the variant Fc region comprises an IgG2 Fc region comprising H268Q, V309L, A330S, and P331S, according to the Kabat numbering system. In some embodiments, the variant Fc region comprises an IgG2 Fc region comprising V234A, G237A, P238S, H268A, V309L, A330S, and P331S, according to the Kabat numbering system. In some embodiments, an antibody comprises a Fc region comprising high mannose glycosylation.

In some embodiments, the variant Fc region comprises an IgG4 Fc region comprising a S228P substitution, according to the Kabat numbering system. In some embodiments, the variant Fc region comprises an IgG4 Fc region comprising an A330S substitution, according to the Kabat numbering system. In some embodiments, the variant Fc region comprises an IgG4 Fc region comprising a P331S substitution, according to the Kabat numbering system.

In some embodiments, the variant Fc region comprises an IgG2 Fc region comprising an A330S substitution, according to the Kabat numbering system. In some embodiments, the variant Fc region comprises an IgG2 Fc region comprising an P331S substitution, according to the Kabat numbering system. In some embodiments, the variant Fc region comprises an IgG2 Fc region comprising an 234A substitution, according to the Kabat numbering system. In some embodiments, the variant Fc region comprises an IgG2 Fc region comprising an 237A substitution, according to the Kabat numbering system.

In some embodiments, the variant Fc region comprises IgG1 Fc region, and wherein the one or more mutations comprises (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (l) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (11) 380D, (mm) 382D or 382P, (nn) 385P, (00) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (111) A330L, (mmm) P331A or P331S, or (nnn) E233P, (000) L234A, L235E, G237A, A330S, and P331S or (ppp) any combination of (a)-(uu), per Kabat numbering.

In some embodiments, the variant Fc region comprises an amino acid sequence as set forth in SEQ ID NO: 311. In some embodiments, the composite binding molecule the CD19 antigen binding component comprises a heavy chain immunoglobulin sequence set forth in SEQ ID NO: 301 or 304, and the CD38 binding component comprises a heavy chain immunoglobulin sequence set forth in SEQ ID NO: 302, 303, 305-310.

### Framework Region

Mutations or reversions to a germline sequence made within the framework regions of the heavy and light chains can be advantageous for improving the pharmacokinetic and pharmacodynamic properties of the CD19 and CD38 binding molecules described herein. In certain instances, mutations or reversions to a germline sequence made within a of the heavy and/or light chain improve stability of the CD19 and CD38 binding molecules (e.g. the bispecific antibodies described herein). In certain instances, mutations or reversions to a germline sequence made within a of the heavy and/or light chain reduce immunogenicity of the CD19 and CD38 binding molecules (e.g. the bispecific antibodies described herein). Accordingly, in some embodiments, a Framework Region of a heavy chain and/or light chain comprises 1, 2, 3, 4 5, 8, or 10 mutations or reversions back to a germline sequence. In some embodiments, the Framework Region of a heavy chain and/or light chain comprises 1 mutation or reversion back to a germline sequence to 10 mutations or reversions back to a germline sequence. In some embodiments, the Framework Region of a heavy chain and/or light chain comprises at least 1 mutation or reversion back to a germline sequence. In some embodiments, the Framework Region of a heavy chain and/or light chain comprises at most 10 mutations or reversions back to a germline sequence. In some embodiments, the Framework Region of a heavy chain and/or light chain comprises 1 mutation or reversion back to a germline sequence to 2 mutations or reversions back to a germline sequence, 1 mutation or reversion back to a germline sequence to 3 mutations or reversions back to a germline sequence, 1 mutation or reversion back to a germline sequence to 4 mutations or reversions back to a germline sequence, 1 mutation or reversion back to a germline sequence to 5 mutations or reversions back to a germline sequence, 1 mutation or reversion back to a germline sequence to 8 mutations or reversions back to a germline sequence, 1 mutation or reversion back to a germline sequence to 10 mutations or reversions back to a germline sequence, 2 mutations or reversions back to a germline sequence to 3 mutations or reversions back to a germline sequence, 2 mutations or reversions back to a germline sequence to 4 mutations or reversions back to a germline sequence, 2 mutations or reversions back to a germline sequence to 5 mutations or reversions back to a germline sequence, 2 mutations or reversions back to a germline sequence to 8 mutations or reversions back to a germline sequence, 2 mutations or reversions back to a germline sequence to 10 mutations or reversions back to a germline sequence, 3 mutations or reversions back to a germline sequence to 4 mutations or reversions back to a germline sequence, 3 mutations or reversions back to a germline sequence to 5 mutations or reversions back to a germline sequence, 3 mutations or reversions back to a germline sequence to 8 mutations or reversions back to a germline sequence, 3 mutations or reversions back to a germline sequence to 10 mutations or reversions back to a germline sequence, 4 mutations or reversions back to a germline sequence to 5 mutations or reversions back to a germline sequence, 4 mutations or reversions back to a germline sequence to 8 mutations or reversions back to a germline sequence, 4 mutations or reversions back to a germline sequence to 10 mutations or reversions back to a germline sequence, 5 mutations or reversions back to a germline sequence to 8 mutations or reversions back to a germline sequence, 5 mutations or reversions back to a germline sequence to 10 mutations or reversions back to a germline sequence, or 8 mutations or reversions back to a germline sequence to 10 mutations or reversions back to a germline sequence. In some embodiments, the Framework Region of a heavy chain and/or light chain comprises 1 mutation or reversion back to a germline sequence, 2 mutations or reversions back to a germline sequence, 3 mutations or reversions back to a germline sequence, 4 mutations or reversions back to a germline sequence, 5 mutations or reversions back to a germline sequence, 8 mutations or reversions back to a germline sequence, or 10 mutations or reversions back to a germline sequence. In some embodiments, the CD38 binding moiety comprises a heavy chain framework region as set forth in SEQ ID NO: 5. In some embodiments, the CD binding moiety comprises a heavy chain framework region as set forth in SEQ ID NO: 6 or 7.

### Pharmaceutically Acceptable Excipients, Carriers, and Diluents

Compositions comprising the composite binding molecules of the current disclosure are included in a pharmaceutical composition comprising one or more pharmaceutically acceptable excipients, carriers, and diluents. In certain embodiments, the antibodies of the current disclosure are administered suspended in a sterile and/or isotonic solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

Subcutaneous formulations for administration of antibodies can comprise one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA. Additionally, a compound or molecule that relieves pain at the injection site can be included, such as hyaluronidase, for example at a concentration of from about 2,000 U/ml to about 12,000 U/ml.

In certain embodiments, the composite binding molecules of the current disclosure are shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

Also described herein are kits comprising one or more of the composite binding molecules described herein in a suitable container and one or more additional components selected from: instructions for use; a diluent, an excipient, a carrier, and a device for administration.

In certain embodiments, described herein is a method of preparing a cancer treatment comprising admixing one or more pharmaceutically acceptable excipients, carriers, or diluents and a composite binding molecule of the current disclosure. In certain embodiments, described herein is a method of preparing a cancer treatment for storage or shipping comprising lyophilizing one or more antibodies of the current disclosure.

### Production and Manufacture

The nucleic acids encoding the composite binding molecules (e.g. bispecific antibodies) described herein can be used to infect, transfect, transform, or otherwise render a suitable cell transgenic for the nucleic acid, thus enabling the production of composite binding molecules for commercial or therapeutic uses. Standard cell lines and methods for the production of antibodies from a large-scale cell culture are known in the art. See e.g., Li et al., “Cell culture processes for monoclonal antibody production.” Mabs. 2010 September-October; 2 (5): 466-477.

In certain embodiments, a nucleic acid sequence encodes the composite binding molecule or bispecific antibodies disclosed herein. In certain embodiments, the polynucleotide sequence encoding the composite binding molecule is operatively coupled to a eukaryotic regulatory sequence. In some embodiments, a cell comprises the nucleic acid sequence.

In some embodiments, a cell comprises a nucleic acid encoding the composite binding molecules disclosed herein. In certain embodiments, the cell comprises a prokaryotic cell. In certain embodiments, the prokaryotic cell is an Escherichia coli cell. In certain embodiments, the cell comprises a eukaryotic cell. In certain embodiments, the eukaryotic cell is a Chines Hamster Ovary (CHO) cell, an NS0 murine myeloma cell, or a human PER. C6 cell

In certain embodiments, described herein is a method of making a composite binding molecule comprising culturing a cell comprising a nucleic acid encoding a composite binding molecule under conditions in vitro sufficient to allow production and secretion of the composite binding molecules.

In certain embodiments, described herein, is a master cell bank comprising: (a) a mammalian cell line comprising a nucleic acid encoding an antibody described herein integrated at a genomic location; and (b) a cryoprotectant. In certain embodiments, the cryoprotectant comprises glycerol. In certain embodiments, the master cell bank comprises: (a) a CHO cell line comprising a nucleic acid encoding a composite binding molecule integrated at a genomic location; and (b) a cryoprotectant. In certain embodiments, the cryoprotectant comprises glycerol. In certain embodiments, the master cell bank is contained in a suitable vial or container able to withstand freezing by liquid nitrogen.

Also described herein are methods of making composite binding molecules described herein. Such methods comprise incubating a cell or cell-line comprising a nucleic acid encoding the composite binding molecules in a cell culture medium under conditions sufficient to allow for expression and secretion of the composite binding molecules, and further harvesting the composite binding molecules from the cell culture medium. The harvesting can further comprise one or more purification steps to remove live cells, cellular debris, non-composite binding molecules proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

### Methods of Use

Suppression of the immune response by immunoregulatory cells can facilitate tumor growth, migration, and metastasis. Immunosuppression or negative immune modulation can include processes or pathways that result in the full or partial reduction of the immune response. Immunosuppression can be systemic or localized to a specific site (e.g. the tumor microenvironment), tissue, or region of a subject's or patient's body. Although B cells are primarily known as a positive immune modulator through the production of antibodies that facilitate neutralization of a pathogen, certain populations of B cells can function to suppress or negatively regulate the immune response. Such populations of B cells can be defined by the expression of more than one cell surface biomarkers. Immunosuppressive B cells or B-cell populations can comprise a B-cell linage surface biomarker and a suppressive B-cell surface biomarker. The B-cell lineage surface markers can comprise CD19, CD138, IgA, or CD45. B-cell surface markers can comprise IgD, CD1, CD5, CD21, CD24, CD38, HM13, SLAMF7, AQP3, or latent TGF-beta (e.g., TGF-beta LAP). Immunosuppressive B cells or immunosuppressive B-cell populations can function to suppress the immune response by suppressing a diverse set of cell subtypes, including T cells, through the secretion of anti-inflammatory mediators, such as cytokines. Immunosuppressive B cells can also function in attenuating the immune response by negatively modulating lymphoid structures and/or facilitating the conversion of T cells to regulatory T cells. Thus, disclosed herein are methods for targeting immunosuppressive B-cell populations to effectively modulate a response.

Targeting immunosuppressive B cells or B-cell populations can result in the immune activation or positive modulation of the immune response against a tumor or tumorigenic cell. Provided herein are methods of treating an individual afflicted with a cancer or a tumor comprising administering to the individual afflicted with the cancer or the tumor the composite binding molecules disclosed herein. Also provided herein are methods of reducing immunosuppressive B cells in, adjacent to, or surrounding a tumor of an individual afflicted with a tumor or cancer comprising administering to the individual afflicted with the tumor or the cancer the composite binding molecules disclosed herein, thereby reducing immunosuppressive B cells in, adjacent to, or surrounding the tumor. Further disclosed are methods of contacting an immunosuppressive B cell in a subject with a composite binding molecule, wherein the method comprises administering the composite binding molecule to the subject. In certain embodiments, the subject has a tumor or cancer.

The type, subtype, or form of a tumor or cancer can be an important factor in treatment strategies and methods. In some embodiments, the cancer or tumor is a solid-tissue cancer. In some embodiments, the cancer comprises breast cancer, prostate cancer, pancreatic cancer, lung cancer, kidney cancer, stomach cancer, esophageal cancer, skin cancer, colorectal cancer, or head and neck cancer.

Immunosuppressive B cells can suppress the anti-tumor immune response. In some embodiments, the tumor or cancer comprise B cells comprising a B-cell linage surface biomarker and a suppressive B-cell surface biomarker. The B-cell lineage surface markers can comprise CD19, CD138, IgA, or CD45. B-cell surface markers can comprise IgD, CD1, CD5, CD21, CD24, CD38, HM13, SLAMF7, AQP3, or TGFB. In some embodiments, the B-cell surface markers comprise CD19 (e.g. CD19+) and CD38 (e.g. CD CD38+). In Some embodiments, the tumor infiltrating B cells or the immunosuppressive B cells comprise CD19+, CD38+B cells.

In certain embodiments, disclosed herein, are bispecific antibodies useful for the treatment of a cancer or tumor associated with CD19, CD38, CD20 negative cancer or tumors. Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

Cancers associated with CD19 positive, CD38 positive, CD20 negative immunosuppressive B cells are those cancers or tumors that have a CD19 positive, CD38 positive population (e.g., tumor infiltrating or adjacent leukocytes) that are at least 10%, 20%, 30%, 40%, 50%, 60%, 70%, or 80% CD20 negative. CD20 negativity can be determined for example by flow cytometry (e.g., no increase in CD20 staining compared to unstained or isotype control stained cells). Conversely the CD19 positive, CD38 positive population can be determined, for example, by flow cytometry (e.g., does show increase in CD19 and CD38 staining compared to unstained or isotype control stained cells). In certain embodiments, the CD19 positive, CD38 positive, CD20 negative B cells express CD30.

In certain embodiments, the CD19 positive, CD38 positive, CD20 negative cancer or tumor is a solid cancer or tumor. In certain embodiments, the cancer or tumor is a blood cancer or tumor. In certain embodiments, the tumor/cancer to be treated with one or more antibodies of the invention comprise brain cancer, head and neck cancer, colorectal carcinoma, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed. In a certain embodiment, the cancer treated is refractory to one or more standard treatments. In a certain embodiment, the cancer is a relapsed/refractory glioblastoma, pancreatic cancer, ovarian cancer, colon cancer, prostate cancer, or lung cancer. In certain embodiments, the cancer or tumor is a blood cancer In certain embodiments, the blood cancer is diffuse large B cell lymphoma. In certain embodiments, the blood cancer is myeloma. In certain embodiments, the blood cancer is Burkitt's lymphoma. In certain embodiments, the blood cancer is aggressive B cell lymphoma. In certain embodiments, the aggressive B cell lymphoma comprises double hit lymphoma, double expressor lymphoma, or triple hit lymphoma. In certain embodiments, the cancer or tumor is one that is PD-L1 or PD-L2 positive. In certain embodiments, the cancer or tumor is one that is PD-L1 positive.

Also described herein are methods of identifying certain cancers associated with CD19 positive, CD38 positive, CD20 negative immunosuppressive B cells for treatment. Such cancers are those associated CD19 positive, CD38 positive, CD20 negative B cells that display a CD38 high phenotype. Such methods involve: a) obtaining a biological sample from an individual (e.g., peripheral blood or tumor); b) performing an assay on the B cells of the biological sample (e.g., peripheral circulating B cells or tumor infiltrating or tumor adjacent B cells); and c) administering a bispecific antibody that binds to CD19 and CD38 to the individual if the B cells exhibit a CD38 high phenotype. In certain embodiments, the method involves: a) obtaining a biological sample from an individual (e.g., peripheral blood or tumor); b) performing an assay on the B cells of the biological sample (e.g., peripheral circulating B cells or tumor infiltrating or tumor adjacent B cells); and c) administering separate antibodies that bind CD19 and CD38 to the individual.

In certain embodiments, described herein is a method of treating an individual afflicted with a tumor or cancer, the method comprising performing an assay on the B cells of a biological sample of the individual for a CD38 high phenotype; and administering a bispecific antibody that binds CD19 and CD38 to the individual afflicted with the tumor or the cancer based on results of the assay on the B cells of a biological sample from the individual. In certain embodiments, the results indicate a CD38 high phenotype in the B cells from the biological sample.

In certain embodiments, described herein is a method of treating an individual afflicted with a tumor or cancer, the method comprising administering a bispecific antibody that binds CD19 and CD38 to the individual afflicted with the tumor or the cancer based on results of an assay on B cells of a biological sample of the individual. In certain embodiments, the results indicate a CD38 high phenotype in the B cells from the biological sample.

A CD38 high phenotype can be suitably determined by the skilled artisan. In certain embodiments, a CD38 high phenotype is determined by an assay on the cell surface expression of CD38 (e.g., flow cytometry, plate assays read by fluorescence plate readers, or microscopy). However, other methods can in certain instances, to the extent that such methods correlate with high surface level expression CD38, be used to determine a CD38 high phenotype (e.g., analyzing levels of mRNA or intracellular or total CD38 protein in a biological sample).

A CD38 high phenotype can be indicated by a percentage of CD38 positive B Cells in the peripheral blood. In certain embodiments, assay results indicate a CD38 high phenotype if greater than about 1%, 1.5%, 2.0%, 2.5%, 3.0%, 3.5%, or 4.0% of CD19+CD20-cells in the peripheral blood are CD38 positive. Such positivity can be determined by flow cytometry or microscopy by comparison to a control (e.g., isotype matched control antibody of fluorescent bead controls). In certain embodiments, the CD38 high phenotype is indicated in a patient with a solid tumor.

A CD38 high phenotype can be indicated by a percentage of CD38 positive B Cells in a biopsy sample of a tumor. In certain embodiments, assay results indicate a CD38 high phenotype if greater than about 10%, 15%, 20%, 25%, 30%, 35%, 40%, 45%, or 50% of CD19+CD20-cells in the peripheral blood are CD38 positive. Such positivity can be determined by flow cytometry or microscopy by comparison to a control (e.g., isotype matched control antibody of fluorescent bead controls). In certain embodiments, the CD38 high phenotype is indicated in a patient with a solid tumor.

A CD38 high phenotype can be indicated by making a determination of absolute numbers of CD38 molecules on the surface of a B cell In certain embodiments, assay results indicate a CD38 high phenotype if greater than about 30,000, 35,000, 40,000, 45,000, 50,000, 55,000, 60,000, 65,000, on average are present on B cells with that are positive for CD19

A CD20 negative phenotype can be identified by lack of detectable expression of CD20 (when compared to isotype control) by a standard assay such as flow cytometry. A CD20 low phenotype can be identified by low levels of expression of CD20 (e.g., less than a mature non-immunosuppressive or regulatory B CD19 positive, CD20 positive B cell). In certain embodiments, the CD20low B cell expresses 2-fold, 3-fold, or 4-fold less cell surface CD20 than a non-regulatory or immunosuppressive B cell.

In embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 5 mg/kg and about 30 mg/kg. Therapeutically effective amounts include amounts are those sufficient to ameliorate one or more symptoms associated with the disease or affliction to be treated.

### EXAMPLES

The following examples are included for illustrative purposes only and are not intended to limit the scope of the invention.

### Example 1: Octet Binding Data

The binding affinities of parental and bispecific antibodies were determined using bio-layer interferometry. Binding experiments were performed on Octet Red96 at 25° C. using an assay Buffer consisting of 0.1% BSA, 1×PBS, 0.02% Tween-20, 0.05% NaN3. The antibodies were loaded onto Anti-hIgG Fc Capture biosensors for 300 seconds. The ligand-loaded sensors were dipped into a series dilution (starting at 300 nM: two-fold series dilution for CD19 and three-fold series dilution for CD38) of the antigens for association (200 seconds for CD19 and 150 seconds for CD38) followed by dissociation (600 seconds for CD19 and 400 seconds for CD38). Kinetic constants were calculated using a monovalent (1:1) binding model.

Parental test articles included:
851A=anti-CD19 3C10
851B=anti-CD19 3C10 heavy chain & anti-CD38 003 light chain
851C=anti-CD38 003 heavy chain & anti-CD19 3C10 light chain
851D=anti-CD38 003
851E=anti-CD19 3C10 (scFv-Fc) 2
851F=anti-CD38 003 (scFv-Fc) 2

The two parental antibodies with anti-CD19 3C10 VH and VL (851A/851E) bound CD19 with a similar KD. Substituting the anti-CD19 3C10 VL with the anti-CD38 VL (851B) resulted in a reduction of binding to CD19 of about 5-fold. The parental antibodies with anti-CD38 003 VH and VL (851D/851F) did not bind to CD19, as expected.

Table 2 shows binding data. The two parental antibodies with anti-CD38 003 VH and VL (851D/851F) bound CD38 with a similar KD. Substituting the anti-CD38 003 VL with the anti-CD19 VL (851C) resulted in a large reduction of binding to CD38. The parental antibodies with anti-CD19 VH and VL (851A/851E) did not bind to CD38, as expected; 851B also did not bind to CD38. This data shows that only the anti-CD38 003 VL can function as a common light chain for the anti-CD193C10 VH.

TABLE 2

| | KD (nM) | |
|---|---|---|
| Sample | CD19 | CD38 |
| 851A | 1.53 | NB |
| 851B | 7.07 | NB |
| 851C | NB | 285 |
| 851D | NB | 0.98 |
| 851E | 1.21 | NB |
| 851F | NB | 2.22 |

NB = no binding

Bispecific antibody (format) test articles included:
BS1=1:1:2 ratio 003HC: 3C10HC: 003LC (common light chain)
BS1b=2:1:2 ratio 003HC: 3C10HC: 003LC (common light chain)
BS2=1:1:1 ratio 003Knob: 3C10scFvHole: 003LC (Fab-Fc:scFv-Fc bispecific IgG1)
BS2b=4:1:4 ratio 003Knob: 3C10scFvHole: 003LC (Fab-Fc:scFv-Fc bispecific IgG1)
BS3=1:1:1 ratio 3C10scFv-003Fab-FcKnob:FcHole:003LC) (scFv-Fab-Fc:Fc bispecific IgG1)
BS4=1:1:1 ratio 003Fab-FcKnob-3C10scFv:FcHole (Fab-Fc-scFv:Fc bispecific IgG1)
BS4b=4:1:4 ratio 003Fab-FcKnob-3C10scFv:FcHole (Fab-Fc-scFv:Fc bispecific IgG1)
CM1=1:1:2 ratio 3C10Hole:VZVKnob: 003LC anti-CD19 control antibody
CM1b=1:3:3 ratio 3C10Hole:VZVKnob: 003LC
CM2=1:1:2 ratio 003Knob:VZVHole: 003LC anti-CD38 control antibody
CM2b=3:1:3 ratio 003Knob:VZVHole: 003LC

Table 3 shows binding data for bispecific test articles in a single antigen format. Bispecific antibodies BS1/BS2/BS4 bound to both target antigens with a KD within 4-fold of parental antibodies (shown with gray shading). BS3 bound only to CD19 but not CD38 suggesting that either the anti-CD38 Fab binding site was blocked by the anti-CD19 scFv N-terminal fusion or the anti-CD38 requires a free VH N-terminus for binding. One-arm control antibodies (CM1, CM2) bound only to their intended target antigen.

TABLE 3

| | KD (nM) | |
|---|---|---|
| Sample | CD19 | CD38 |
| BS1 | 13.8 | 1.32 |
| BS1b | 13.0 | 1.23 |
| BS2 | 1.53 | 1.33 |
| BS2b | 1.58 | 1.12 |
| BS3 | 3.32 | NB |
| BS4 | 1.31 | 1.20 |
| BS4b | 4.78 | 1.21 |
| CM1 | 18.2 | NB |
| CM1b | 15.9 | 2210 |
| CM2 | NB | 1.67 |
| CM2b | 21300 | 0.59 |

NB = no binding

For a two-antigen format, the antibodies were loaded onto Anti-hIgG Fc Capture biosensors for 300 seconds. The ligand-loaded sensors were saturated with 500 nM of first antigen for 500 seconds followed by 300 nM of second antigen for 240 seconds. Kinetic constants were calculated using a monovalent (1:1) binding model. Table 5 shows bispecific antibodies BS1/BS2/BS4 could simultaneously bind to both target antigens with a ka (1/Ms) within 2-fold of parental antibodies (851B, 851D, and 851E). As with the one-antigen format, BS3 bound only to CD19 but not CD38.

TABLE 4

| | | | Second |
|---|---|---|---|
| | | | Antigen |
| | KD (nM) | | Ka |
| Sample | Antigen 1 | Antigen 2 | (1/Ms)E + 04 |
| 851B | — | CD19 | 4.24 |
| 851D | — | CD38 | 43.1 |
| 851E | — | CD19 | 4.89 |
| BS1 | CD19 | CD38 | 27.0 |
| BS1 | Buffer | CD38 | 40.9 |
| BS1b | CD19 | CD38 | 26.3 |
| BS1b | Buffer | CD38 | 31.6 |
| BS2 | CD19 | CD38 | 25.4 |
| BS2 | Buffer | CD38 | 44.7 |
| BS2b | CD19 | CD38 | 22 |
| BS2b | Buffer | CD38 | 32.8 |
| BS3 | CD19 | CD38 | NB |
| BS3 | Buffer | CD38 | NB |
| BS4 | CD19 | CD38 | 37.2 |
| BS4 | Buffer | CD38 | 44.5 |
| BS4b | CD19 | CD38 | 26.7 |
| BS4b | Buffer | CD38 | 39.0 |
| CM1 | CD19 | CD38 | NB |
| CM1 | Buffer | CD38 | NB |
| CM2 | CD19 | CD38 | 30.0 |
| CM2 | Buffer | CD38 | 29.4 |
| BS1 | CD38 | CD19 | 4.44 |
| BS1 | Buffer | CD19 | 5.34 |
| BS1b | CD38 | CD19 | 6.87 |
| BS1b | Buffer | CD19 | 8.98 |
| BS2 | CD38 | CD19 | 5.74 |
| BS2 | Buffer | CD19 | 5.31 |
| BS2b | CD38 | CD19 | 6.66 |
| BS2b | Buffer | CD19 | 8.20 |
| BS3 | CD38 | CD19 | 8.84 |
| BS3 | Buffer | CD19 | 8.93 |
| BS4 | CD38 | CD19 | 2.65 |
| BS4 | Buffer | CD19 | 4.55 |
| BS4b | CD38 | CD19 | 3.29 |
| BS4b | Buffer | CD19 | 4.64 |
| CM1 | CD38 | CD19 | 5.07 |
| CM1 | Buffer | CD19 | 4.73 |
| CM2 | CD38 | CD19 | NB |
| CM2 | Buffer | CD19 | NB |

NB = no binding

Variants were further tested for the ability to bind CD19 and/or CD38. Binding experiments were performed on Octet Red at 25° C. The antibodies were loaded onto anti-hIgG Fc Capture (AHC) biosensors for 300 seconds. The ligand-loaded sensors were dipped into a two-fold series dilution (starting at 300 nM) of the antigens (CD19 and CD38) for 240 seconds of CD19 and 150 seconds of CD38 for association followed by dissociation for 600 seconds of CD19 and 130 seconds of CD38. Kinetic constants were calculated using a monovalent (1:1) binding model. TABLE 5 shows binding of anti-CD38 CDRH2 variants. TABLE 6 shows binding of the CD38 light chain W32H variant. TABLE 7 shows binding of CD19 heavy chain framework mutant A84S A108L.

TABLE 5
Bispecific BS1 anti-CD38 arm CDR-H2 variants
(“RVIPFLGIAN” disclosed as SEQ ID NO: 85)

| | | | | | | | | | | | CD38 |
|---|---|---|---|---|---|---|---|---|---|---|---|
| | | | | | | | | | | | Binding |
| | SEQUENCE | | | | | | | | | | KD (nM) |
| BS1 | R | V | I | P | F | L | G | I | A | N | 1.2 |
| BS1M-1 | . | . | . | . | . | . | . | T | . | . | 13.2 |
| BS1M-3 | . | . | . | . | H | . | . | | . | . | 6.9 |
| BS1M-4 | . | . | . | . | H | . | . | T | . | . | 94.4 |
| BS1M-6 | . | . | . | . | Q | . | . | | . | . | 4.4 |
| BS1M-7 | . | . | . | . | Q | . | . | T | . | . | 60.5 |
| BS1M-2 | . | . | T | . | . | . | . | T | . | . | No binding |
| BS1M-5 | . | . | T | . | H | . | . | T | . | . | No binding |
| BS1M-8 | . | . | . | . | H | Q | . | T | . | . | No binding |
| BS1M-9 | . | . | . | . | Q | Q | . | T | . | . | No binding |

TABLE 6
Bispecific BS1 common light chain variant

| | CD38 Binding | CD19 Binding |
|---|---|---|
| | KD (nM) | KD (nM) |
| BSM-10 (W32H) | 77.3 | 39.4 |
| BS1 | 1.2 | 13 |

TABLE 7
Bispecific BS1 anti-CD19 arm framework
variant

| | CD19 Binding |
|---|---|
| | KD (nM) |
| BSM-14 (A84S A108L) | 9.5 |
| BS1 | 13 |

### Example 2: Cell Binding Studies

Cell Binding Studies Protocol: Five cell lines (HEK293-CD19, HEK293-CD38, HEK293-CD19/CD38, Daudi, and REH) were incubated with test articles at 133 nM followed by a 3-fold dilution series (7 points total), in addition to a no treatment control, in triplicate. The HEK293 cell lines were transiently transfected.

A study was performed to evaluate the cell surface expression of CD19 and CD38 on Daudi, Raji and REH cell lines. Cells were stained, in triplicate, with commercially available antibody conjugated to PE, washed, and acquired via flow cytometry. To quantify the molecule expression on the surface of the cells, a Quantum Simply Cellular anti-mouse IgG kit from Bangs Laboratories (Catalog #815-A) was used to generate a standard curve for interpolating MFI to a molecule number per cell value (Table 8).

TABLE 8

| | Number of Molecules | |
|---|---|---|
| Cell Line | CD19 | CD38 |
| Daudi | 200,000 | 1,000,000 |
| Raji | 200,000 | 1,000,000 |
| REH | 50,000 | 300,000 |

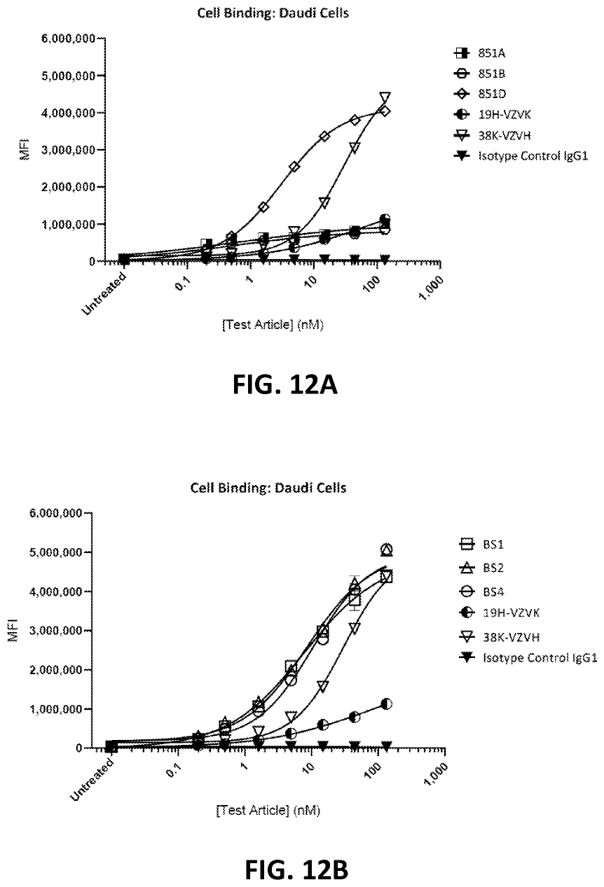

FIG. 12A shows binding to Daudi cells of the parental antibodies (851A, 851B, 851D) and the two control bispecific antibodies (each with one arm against CD19 or CD38 and the other arm against varicella zoster virus). Given that the Daudi cells have ˜1 million copies of CD38 on their surface but only ˜200,000 copies of CD19, FIG. 12A shows efficient binding of anti-CD38 851D and 38K-VZVH but only moderate binding of the anti-CD19 851A, 851B, 19H-VZVK. Note that 851D with two CD38 binding Fabs binds about 5-fold better than 38K-VZVH, which has only one binding Fab for CD38.

FIG. 12B shows binding to Daudi cells of bispecific antibodies BS1, BS2 and BS4. The avidity of the bispecific antibodies, binding to both CD38 and CD19, is apparent by comparing their binding to the 38K-VZVH, which binds only to CD38.

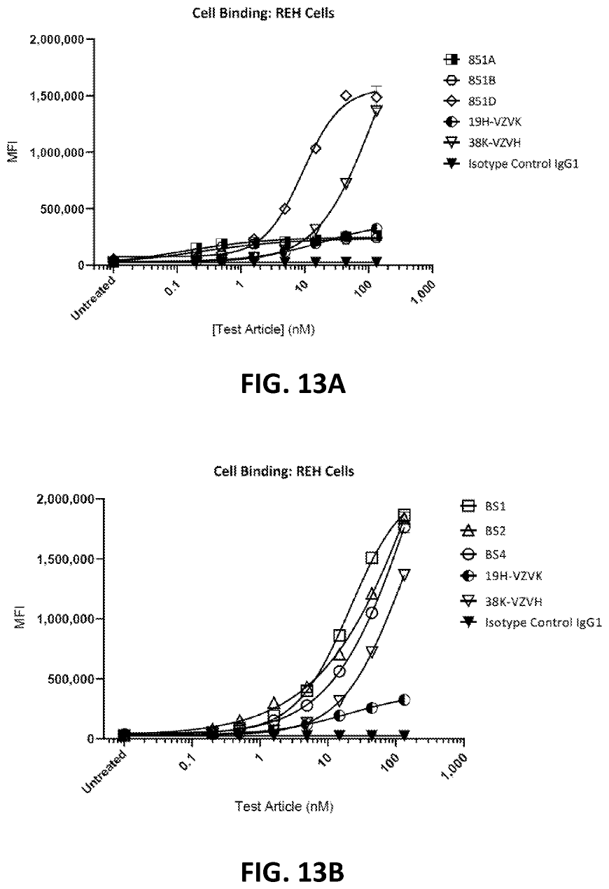

FIG. 13A shows binding to REH cells of the parental antibodies (851A, 851B, 851D) and the two control bispecific antibodies (each with one arm against CD19 or CD38 and the other arm against varicella zoster virus). Given that the REH cells have ˜300,000 copies of CD38 on their surface but only ˜50,000 copies of CD19, FIG. 13A shows efficient binding of anti-CD38 851D and 38K-VZVH but only moderate binding of the anti-CD19 851A, 851B, 19H-VZVK. The magnitude of MFI is significantly less compared to Daudi cells (FIGS. 2A, 2B) due to the lower expression level of both CD38 and CD19 on REH cells. Note that 851D with two CD38 binding Fabs binds about 5-fold better than 38K-VZVH, which has only one binding Fab for CD38.

FIG. 13B shows binding to REH cells of bispecific antibodies BS1, BS2 and BS4. The avidity of the bispecific antibodies, binding to both CD38 and CD19, is apparent by comparing their binding to the 38K-VZVH, which binds only to CD38.

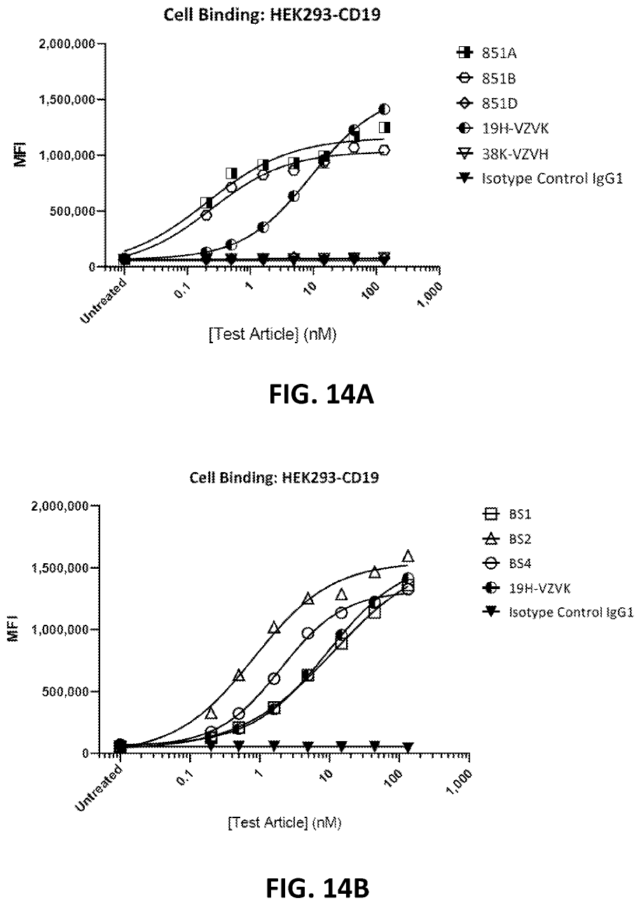

FIG. 14A shows binding to CD19-transfected HEK293 cells of the parental antibodies (851A, 851B, 851D) and two control bispecific antibodies (38K-VZVH, 19H-VZVK). As expected, the two anti-CD38 antibodies do not bind to these cells. Note that 851A and 851B, each with two CD19 binding Fabs, bind significantly better than 19H-VZVK, which has only one binding Fab for CD19.

FIG. 14B shows binding to CD19-transfected HEK293 cells of bispecific antibodies BS1, BS2 and BS4. BS2 and BS$ bind slightly better than BS1; BS2 and BS4 bind CD19 about 10-fold better than BS1 since BS1 has the anti-CD38 light chain (see Table Octet data).

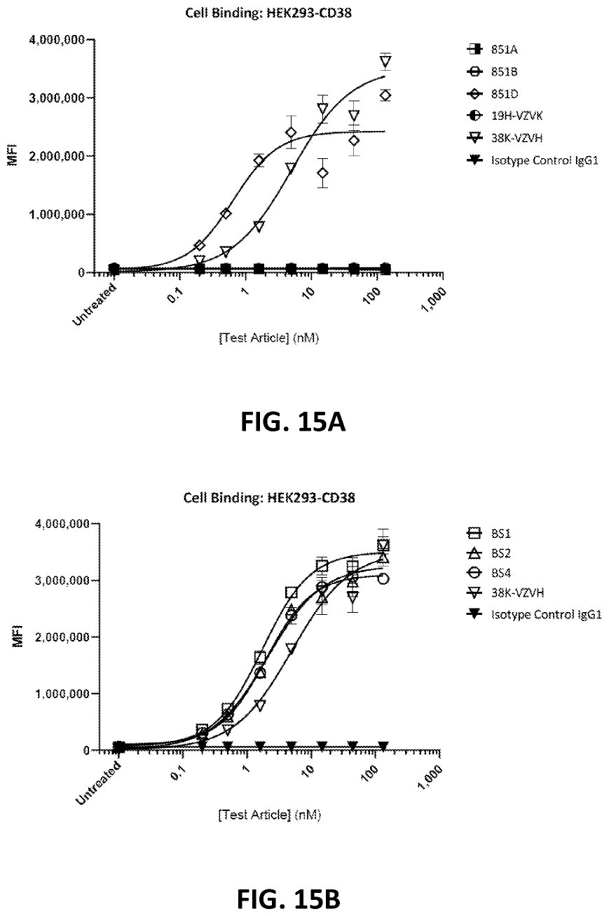

FIG. 15A shows binding to CD38-transfected HEK293 cells of the parental antibodies (851A, 851B, 851D) and two control bispecific antibodies (38K-VZVH, 19H-VZVK). As expected, the three anti-CD19 antibodies do not bind to these cells. Note that 851D, with two CD38 binding Fabs, binds better than 38K-VZVH, which has only one binding Fab for CD38.

FIG. 15B shows binding to CD38-transfected HEK293 cells of bispecific antibodies BS1, BS2 and BS4.

Cell Binding Studies Protocol-Non-Specific Background Binding: A study was performed to evaluate the binding of three parental monoclonal antibodies (anti-CD19 clones 851A and 851B and anti-CD38 clone 851D), a human IgG1 isotype control, and daratumumab to CHO-S and Expi293T cell lines. The two cell lines were stained with a viability dye, then incubated with test articles at a top concentration of 1,250 nM followed by a 5-fold dilution series (4 points total), in addition to a no treatment control, as well as a no treatment, no secondary control, in triplicate.

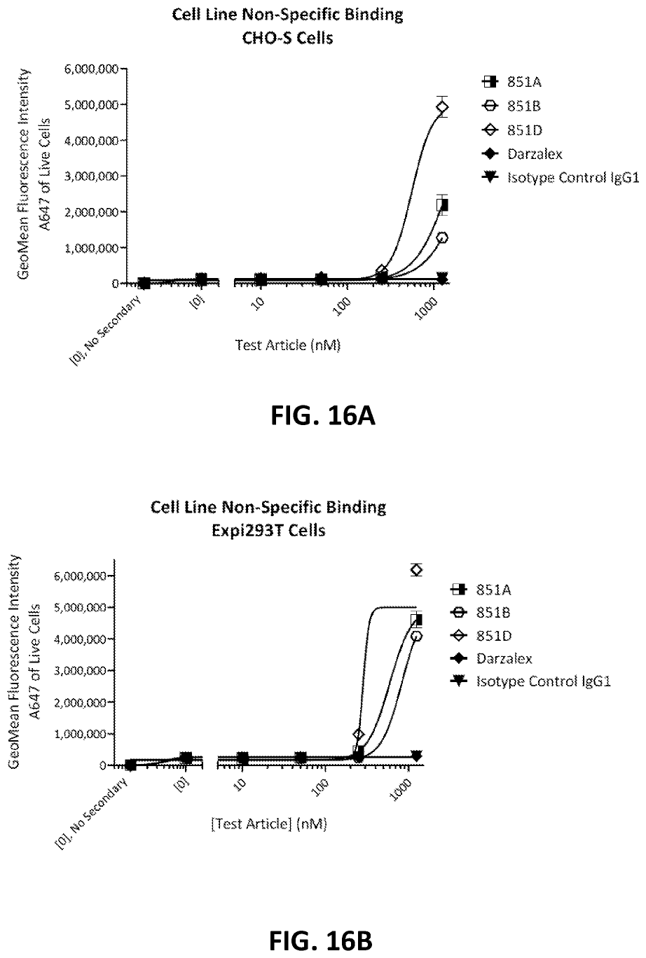

FIG. 16A shows binding to non-transfected CHO-S cells of the parental antibodies (851A, 851B, 851D). Non-specific binding was seen beginning at 250 nM for all three parental antibodies and was more pronounced for anti-CD38 851D.

FIG. 16B shows binding to non-transfected Expi293T cells of the parental antibodies (851A, 851B, 851D). Non-specific binding was seen beginning at 250 nM for all three parental antibodies and was more pronounced for anti-CD38 851D.

### Example 3: Direct and Cross-Linked Apoptosis

For assessment of direct apoptosis, cells were treated with test articles and incubated for 48 hours at 37° C./5% CO2. For assessment of cross-linking induced apoptosis, cells were incubated with test articles on ice for 30 minutes prior to the addition of rabbit anti-human Fc gamma specific F(ab′)₂ at 5 μg/mL. Cells were then incubated for 48 hours at 37C/5% CO2. After incubation, cells were washed and stained with Annexin V, then resuspended in Annexin V buffer containing a viability dye (propidium iodide; PI) prior to flow cytometry acquisition. Early apoptotic cells were defined as Annexin V+/PI− single cells, while late apoptotic/necrotic cells were defined as Annexin V+/PI+ single cells. The sum of Annexin V+/PI− and Annexin V+/PI− were defined as total apoptotic/necrotic cells. The percentages of Annexin V+/PI− cells or Annexin V+/PI+ were plotted to compare the various apoptosis conditions.

For direct apoptosis assessment, test articles were each tested at a final top concentration of 33 nM, followed by a 7-point five-fold dilution series, in addition to an untreated control, in triplicate. For cross-linking induced apoptosis, individual test articles (BS1, BS2, BS4, 851A, 851B, and 851D) and combinations of test articles (851A and 851D; 851B and 851D; and 38K-VZVH and 19H-VZVK), in addition to daratumumab and IgG1 isotype control, were each tested at a final top concentration of 33 nM, followed by a 7-point five-fold dilution series, in addition to an untreated control, in triplicate. As a positive control for Annexin V staining, cells were treated with 5 mM staurosporine.

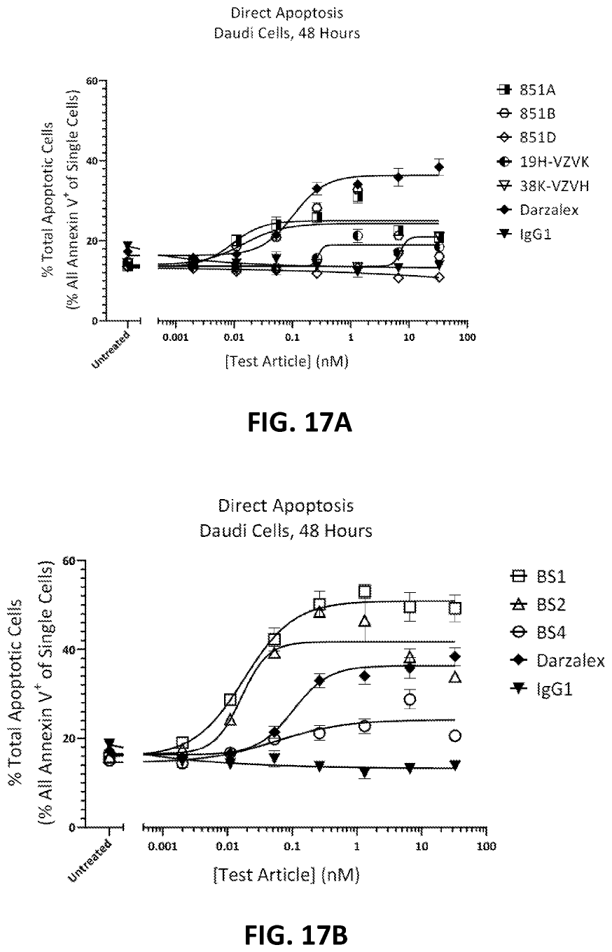

FIG. 17A shows direct apoptosis on Daudi cells for the parental antibodies (851A, 851B, 851D), two control bispecific antibodies (38K-VZVH, 19H-VZVK), daratumumab and IgG1 isotype control. Daratumumab exhibited the highest level of apoptosis. Both anti-CD19 parents (851A, 851B) exhibited a lower level of apoptosis compared to daratumumab. The two bispecific controls and the anti-CD38 parental antibody 851D did not show appreciable direct apoptosis.

FIG. 17B shows direct apoptosis on Daudi cells for bispecific antibodies BS1, BS2, BS4, daratumumab and IgG1 isotype control. BS1 and BS2 formats showed a significantly higher level of direct apoptosis compared to daratumumab. Bispecific format BS4 showed a level of direct apoptosis comparable to the parental anti-CD19 851A/851B antibodies (compare to FIG. 17A); this may be due to the BS4 format not being able to bring the CD19 and CD38 into close proximity in order to initiate apoptosis.

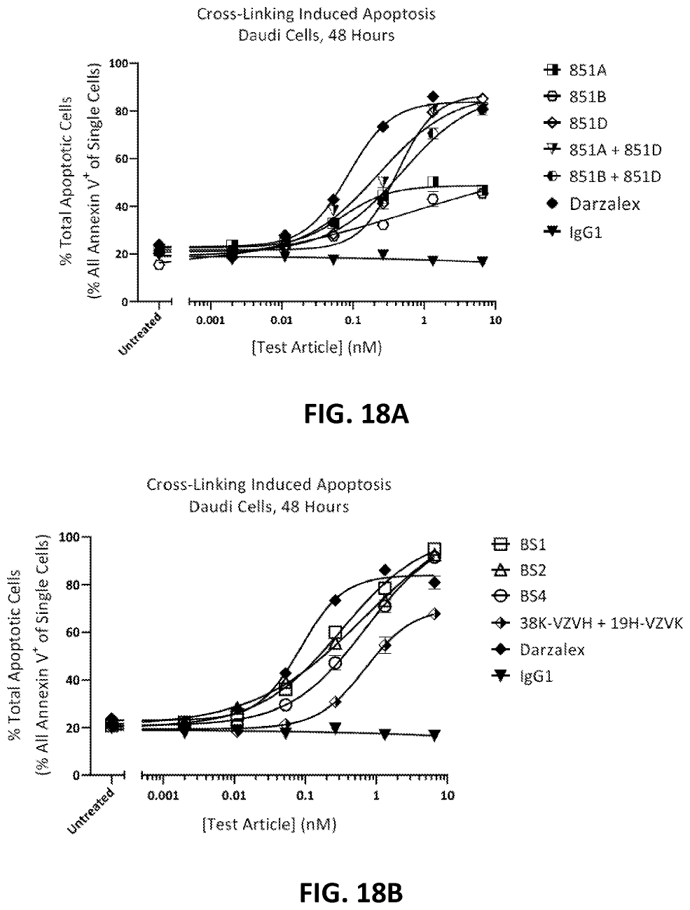

FIG. 18A shows cross-linking induced apoptosis on Daudi cells for the parental antibodies (851A, 851B, 851D), two combinations of parental antibodies (851A+851D; 851B+851D), daratumumab and IgG1 isotype control. Cross-linking increased the level of daratumumab-driven apoptosis. Cross-linking significantly increased the level of apoptosis for anti-CD38 851D, which showed no direct apoptosis. The increase in level of apoptosis when cross-linking the anti-CD19 parent antibodies 851A and 851B was less than for CD38 antibodies, possibly due to the lower level of CD19, compared to CD38, on Daudi cells (see Table 9). Cross-linking combinations of anti-CD19 851A or 851B with anti-CD38 851D did not increase the level of apoptosis compared to 851D alone.

FIG. 18B shows cross-linking induced apoptosis on Daudi cells for bispecific antibodies BS1, BS2, BS4, (38K-VZVH+19H-VZVK), daratumumab and IgG1 isotype control. When cross-linked, BS1 and BS2 formats showed a level of apoptosis comparable to daratumumab. Notably, bispecific format BS4 showed a level of cross-linking induced apoptosis comparable to BS1, BS2 and daratumumab; without cross-linking, BS4 showed no apoptosis (see FIG. 6B). The combination of the two control antibodies, 38K-VZVH and 19H-VZVK, exhibited significant apoptosis but less than any of the bispecific formats, showing that including the anti-CD19 and anti-CD38 binding sites in a single antibody is more advantageous than in independent antibodies.

### Example 4: Cytotoxicity

Daudi target cells were treated with a dose response of test articles and incubated for 15 minutes at 37C/5% CO2. Test articles were tested at a final top concentration of 133 nM, followed by a 7-point five-fold dilution series, in addition to 0 nM control. Daratumumab and IgG1 isotype control were used as a positive and negative control.

Pre-treated target cells were co-cultured with human PBMCs from n=3 donors (E:T 25:1). PBMCs had been “primed” overnight with 100 U/mL of IL-2. PBMCs were ViaFluor 405 (VF405)-labeled. Samples were incubated for 4 hours at 37C/5% CO2 prior to flow cytometry analysis for cytotoxicity. For cytotoxicity analysis, cells were stained with Propidium Iodide (P.I.) and analyzed by high throughput flow cytometry. The percentage of P.I.+ cells within the VF405-population was analyzed as a measure of target cell cytotoxicity.

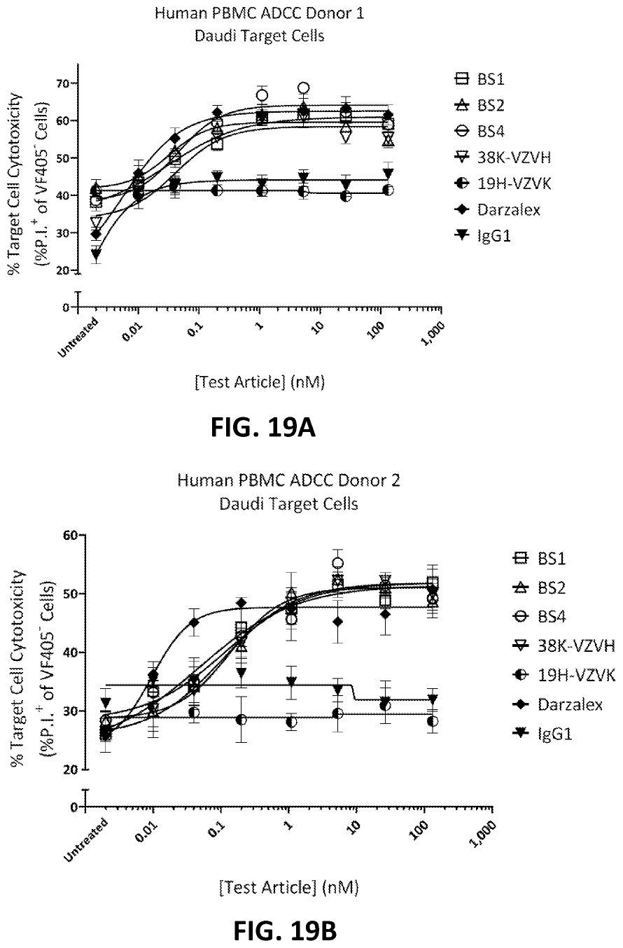

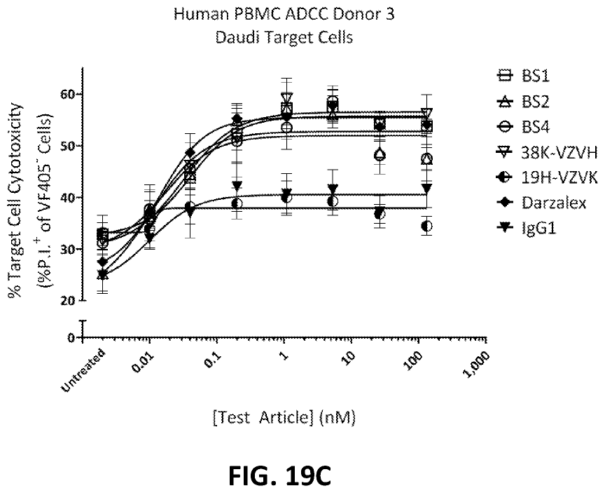

FIGS. 19A, 19B, and 19C show Antibody-Dependent Cellular Cytotoxicity (ADCC) for three donors. For all three donors, the results were similar. The three bispecific formats—BS1, BS2, BS4—and daratumumab exhibited similar levels of ADCC. The anti-CD19 bispecific control 19H-VZVK did not induce ADCC and was equivalent to the IgG1 control antibody, possibly due to low levels of CD19 on the target Daudi cells (see Table 9). In contrast, the anti-CD38 bispecific control 38K-VZVH exhibited ADCC equivalent to the bispecifics and daratumumab, likely due to the much higher level of CD38 on the Daudi cells compared to CD19.

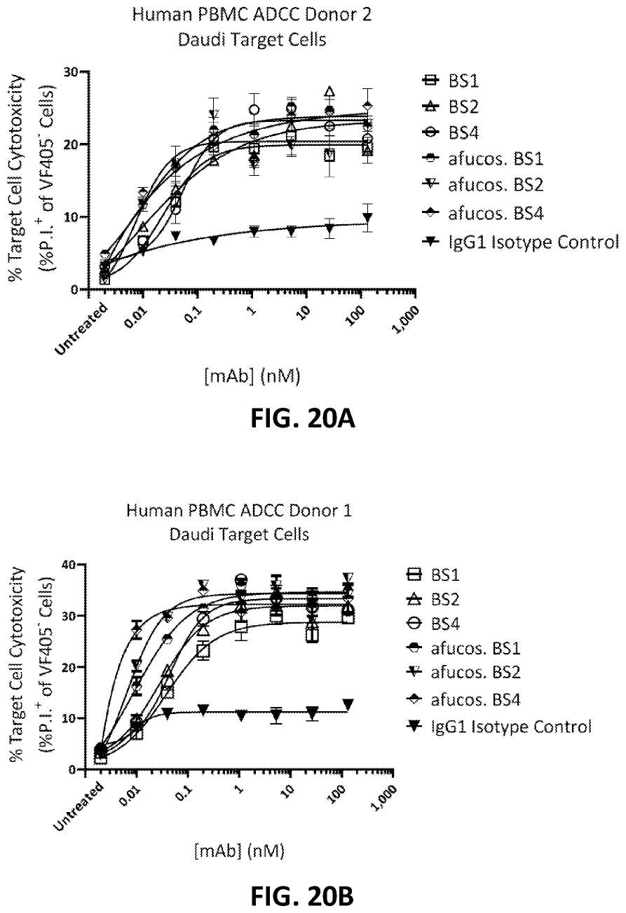

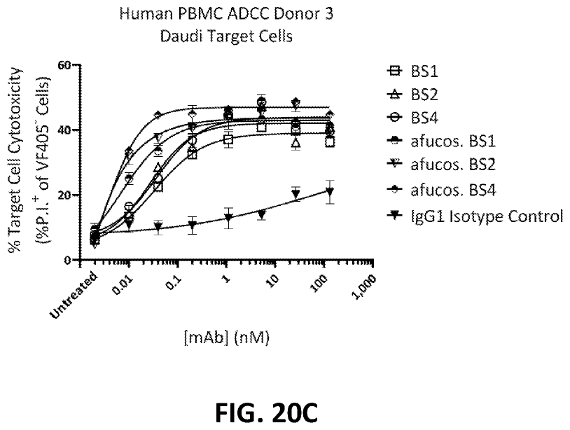

FIGS. 20A. 20B, and 20C show ADCC for three donors. For all three donors, the results were similar. The three bispecific formats—BS1, BS2, BS4—exhibited similar levels of ADCC. Afucosylated versions of BS1, BS2, BS4 showed increased ADCC of about 10-fold compared to the fucosylated versions.

Complement-Dependent Cytotoxicity (CDC) assays were also performed. Target cells were treated with a dose response of the following test articles: BS1, BS2, 38K-VZVH, 19H-VZVH, 38K-VZVH/19H-VZVH combination, as well as controls of Darzalex, anti-CD20, WT IgG1 Tafasitimab, and human IgG1 isotype control. All were tested at a top concentration of 133 nM, followed by a five-fold dilution series, 7 points total, in addition to no treatment controls. After 15 minutes of incubation at 37C, 5% CO2, complement was added to treated cells at a final concentration of 25%. Cells were then incubated with complement for an additional 2 hours at 37C, 5% CO2. After complement incubation, cells were washed and resuspended with 5 μg/mL of a viability dye, propidium iodide (P.I.), and acquired via high throughput flow cytometry.

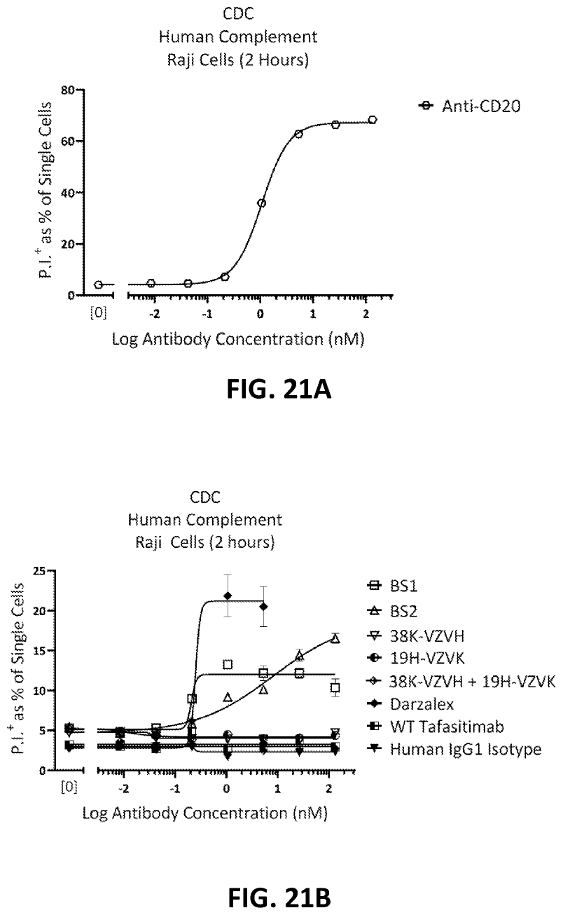

FIGS. 21A and 21B show results of complement-dependent cytotoxicity (CDC) assays. The positive technical control, anti-CD20, induced robust, dose-dependent CDC activity. 38K-VZVH and 19H-VZVH (either alone or in combination), anti-CD19 tafasitimab (wt IgG1), and human IgG1 isotype control did not induce any CDC activity. Darzalex, BS1, and BS2 all showed CDC activity (though not to the same magnitude as anti-CD20, which is expected from the literature). The maximum cytotoxicity of Darzalex was higher than that of both BS1 and BS2.

Antibody-dependent cellular phagocytosis (ADCP) was further assayed by pHrodo Green AM (pHG) labeled Raji cells treated with a dose response of test articles and incubated for 15 minutes at 37C, 5% CO2. pHG is a pH sensitive dye, only weakly fluorescent at neutral pH, but highly fluorescent at low pH in the mature phagosomes of macrophages. pHG labeled Raji target cells with anti-CD20 antibody and IgG1 isotype control were used as a positive control and negative control, with a top concentration of 133 nM, 7-point five-fold dilution series, and 0 nM control. Pre-treated target cells were co-cultured with human macrophages (in vitro differentiated from monocytes) from n=3 donors (E:T1:2). Macrophages were labeled with Cell Trace Violet (CTV). Samples were incubated for 4 hours at 37C, 5% CO2 prior to flow cytometry analysis for phagocytosis. The percentage of pHGhi/CTV+ cells was analyzed as a measure of target cell phagocytosis. Percentages were plotted on an XY chart against the log of the test article concentration, and the data fit to a four-parameter non-linear regression curve from which the EC50 was calculated.

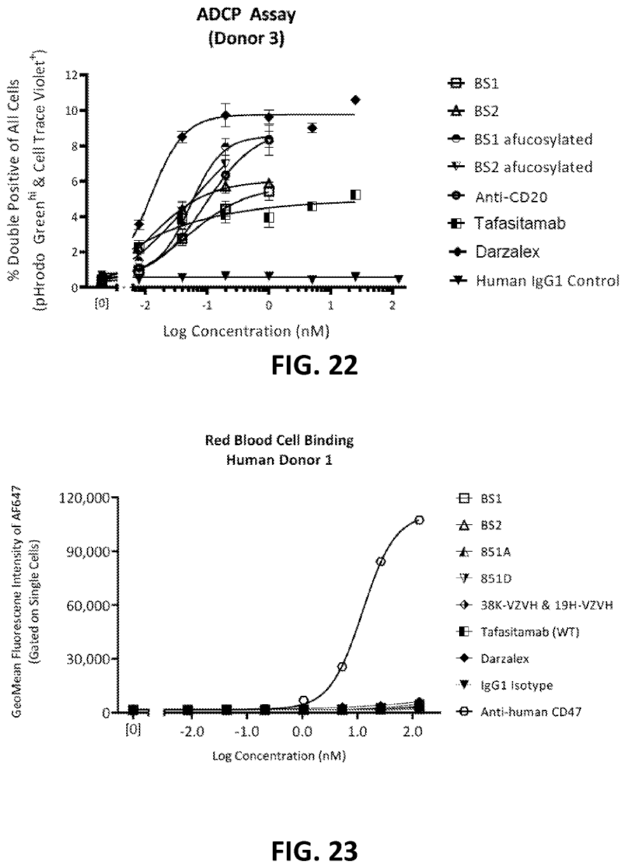

FIG. 22 shows results of antibody-dependent cellular phagocytosis (ADCP) assays using Raji cells as target and donor macrophages. The positive control, anti-CD20, demonstrated dose-dependent phagocytosis for all three of the donors after 4 hours (between 5-10% max phagocytosis). The negative control, IgG1 isotype control, demonstrated no dose-dependent phagocytosis for all three of the donors after 4 hours. Darzalex demonstrated dose-dependent phagocytosis for all three of the donors after 4 hours (between 4-10% max phagocytosis). BS-1, BS-2, afucosylated BS-1, and afucosylated BS-2 showed slight dose-dependent phagocytosis, with afucosylated formats resulting in an increase in ADCP.

### Example 5: Interactions with RBCs

A flow-cytometry based Red Blood Cell (RBC) binding study was performed to evaluate binding of test articles to red blood cells from n=3 cynomolgus monkey and n=3 human donors. Whole blood was washed with 1×PBS and then diluted 20-fold with PBS, prior to treatment with test articles. Bispecifics (BS1, BS2), parental monoclonals (851A, 851D) and controls (anti-CD38 Darzalex, recombinant anti-CD19 tafasitamab, IgG1 isotype control, anti-CD47 conjugated to Alexa Fluor 647) were tested at a top final concentration of 133 nM followed by a five-fold serial dilution of seven points total, in addition to 0 nM control, in triplicate. Single-arm controls (38K-VZVH, 19H-VZVK) were tested in combination, with both at a top concentration of 133 nM and the same dose response.

After incubation with primary antibodies for 30 minutes on ice, cells were washed and stained with 5 μg/mL of a secondary antibody (goat anti-human Fcγ F(ab′)₂ labeled with Alexa Fluor 647) to detect test article binding on red blood cells. Secondary was not used for anti-CD47-A647 stained cells. After incubation with secondary for an additional 30 minutes on ice, stained cells were washed, diluted, and acquired by high-throughput flow cytometry. The AlexaFluor 647 GeoMean Fluorescence Intensity (MFI) of the single cell population was calculated. MFI of AF647 was plotted on an XY chart, graphing MFI against the log of the concentration, and the data fit to a non-linear regression curve from which the EC50 was calculated.

FIG. 23 shows that AF647-conjugated anti-CD47 showed a dose-response binding curve with all three human donors of red blood cells. Darzalex also showed a dose-dependent increase in binding with all three donors, although the maximum MFI was an order of magnitude less than anti-CD47. Anti-CD38 851D showed the next highest maximum MFI, after Darzalex, followed by BS1, BS2, 38K-VZVH & 19H-VZVK together, and anti-CD19 tafasitamab. Finally, anti-CD19 851A and IgG1 isotype showed only a slight increase in MFI at the highest concentration only.

An in vitro hemagglutination assay was performed on red blood cells from a total of three healthy (n=3) cynomolgus monkey (Cyno) donors and three healthy (n=3) human donors. Whole blood was acquired the day of the study and inspected for coagulation. Blood was then washed with PBS and diluted 1:50 to obtain the “whole blood substrate”. Whole blood substrate was plated in 96-well round bottom plates and treated with test articles (BS1, BS2, 38K-VZVH+19H-VZVK, 851A, and 851D), controls (tafasitimab with wild-type IgG1), Darzalex, and human IgG1 isotype control), or a positive technical control (IGM-55.5), in PBS at a top final concentration of 133 nM followed by a five-fold serial dilution of six points, in addition to 0 nM control, in triplicate. After 1 hour of incubation at 37C, 5% CO2, the plate(s) were photographed to ascertain the level of hemagglutination. Each well was scored on a specific hemagglutination scale from 0-5, using the photographs as a reference. The specific manifestation of each score is somewhat relative to the individual donor.

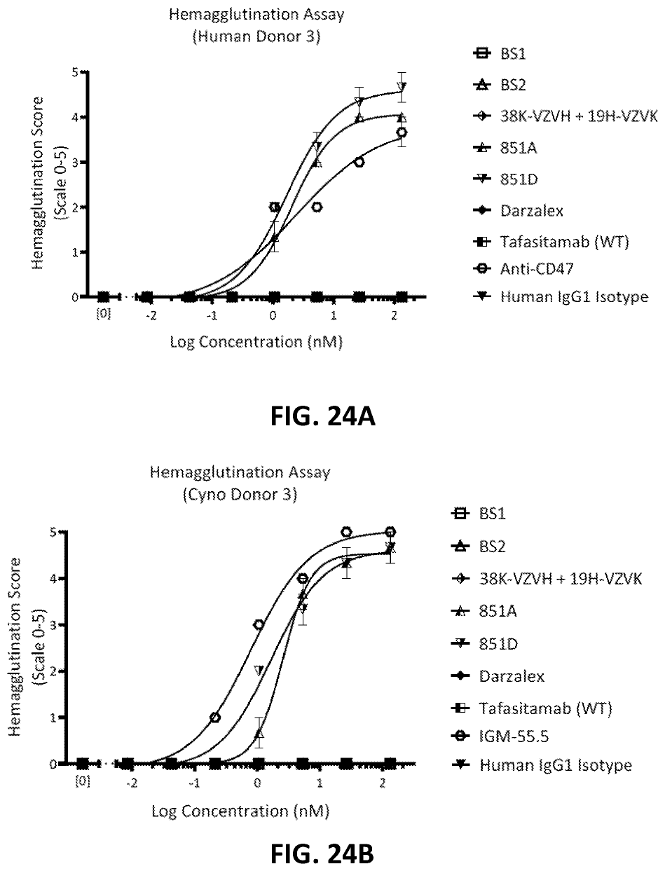

FIG. 24A shows results of the hemagglutination assay for human donor 3. The positive control, anti-CD47, induced hemagglutination for all three human donors, starting between 0.04 and 1.1 nM. BS1, BS2, 38K-VZVH+19H-VZVK, Darzalex, tafasitimab, and human IgG1 isotype control all showed no induction of hemagglutination at any concentration for all three donors. Monoclonal antibodies 851A (anti-CD19) and 851D (anti-CD38) both induced hemagglutination for all three donors, starting at 0.2 or 1.1 nM for each, with a response similar in magnitude to the technical control (anti-CD47). In contrast to the parent monoclonal antibodies, BS1 and BS2 did not show any induction of hemagglutination at any concentration.

FIG. 24B shows results of the hemagglutination assay for cynomolgus donor 3. The positive control, IGM-55.5 (anti-little i antigen IgM antibody) induced hemagglutination for all three cyano donors starting at 0.04 or 0.2 nM. BS1, BS2, 38K-VZVH+19H-VZVK, Darzalex, tafasitimab, and human IgG1 isotype control all showed no induction of hemagglutination at any concentration for all three donors. Monoclonal antibodies 851A (anti-CD19) and 851D (anti-CD38) both induced hemagglutination for all three donors, starting at 1.1 nM for each. In contrast to the parent monoclonal antibodies, BS1 and BS2 did not show any no induction of hemagglutination at any concentration.

An in vitro hemolysis assay was also performed on red blood cells from three (n=3) healthy cynomolgus monkey (cyano) and three (n=3) healthy human donors. Whole blood was acquired the day of the study and inspected for coagulation. Blood was washed with PBS and diluted 1:10 to obtain the “whole blood substrate”. The whole blood substrate was treated with test articles and controls in PBS. Bispecifics (BS1, BS2), parental monoclonals (851A, 851D) and controls (anti-CD38 Darzalex, recombinant anti-CD19 Tafasitamab, IgG1 isotype control) were tested at a top final concentration of 133 nM followed by a five-fold serial dilution of seven points total, in addition to 0 nM control, in triplicate. Single-arm controls (38K-VZVH, 19H-VZVK) were tested in combination, with both at a top concentration of 133 nM and the same dose response. Saponin was tested at a top concentration of 0.1% with a three-fold serial dilution of seven points total. After 1 hour of incubation at 37C, 5% CO2, plates were centrifuged, and supernatant was collected. Supernatant was analyzed via plate reader for optical density (OD) at 540 nm. The positive control, Saponin, induced dose-dependent hemolysis starting at 0.001% thru 0.10%, for all species and donors. No test articles induced any hemolysis at any concentration tested.

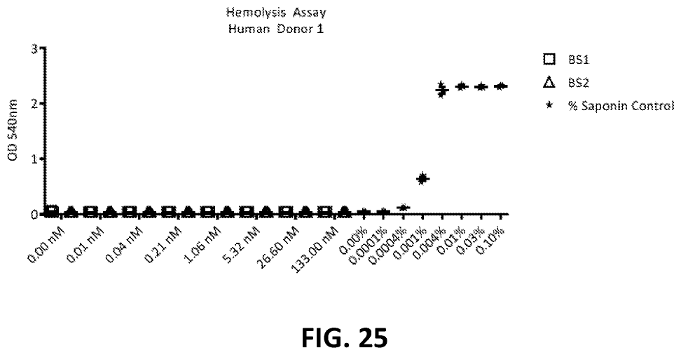

FIG. 25 shows that none of the test articles induced any hemolysis at any concentration tested. The positive control, Saponin, induced dose-dependent hemolysis starting at 0.001% thru 0.10%, for all species and donors.

### Example 6: FcR Variant Lowers ADCC in a CD38 and CD19 Binding Bispecific Antibody

B cells isolated from healthy Human peripheral blood mononuclear cells (PBMCs) were treated with a dose response of test articles and incubated for 15 mins. Raji and Daudi target cells were also treated with a dose response of Rituxan, Darzalex, or IgG1 isotype controls and incubated for 15 minutes at 37C, 5% CO2. N=5 test articles and n=3 controls (Rituxan, Darzalex, and Human IgG1 isotype) were tested at a final top concentration of 133 nM, followed by a seven point five-fold dilution series, in addition to 0 nM control.

Pre-treated target cells were co-cultured with human PBMCs from n=3 donors (E:T 25:1). PBMCs were primed overnight with 100 U/mL of IL-2. PBMCs were ViaFluor 405-labeled. Samples were incubated for 4 hours at 37C, 5% CO2. Test articles included BS1, BS1 afucosylated, BS1 having a Fc variant (“dead Fc”, e.g., SEQ ID NOs: 301 and 302), and controls Rituxan, Darzalex, and Human IgG1 isotype. BS1 already exhibited a favorably low ADCC profile, therefore it was interesting in that in each case, the variant Fc (dead Fc) further lowered and/or reduced

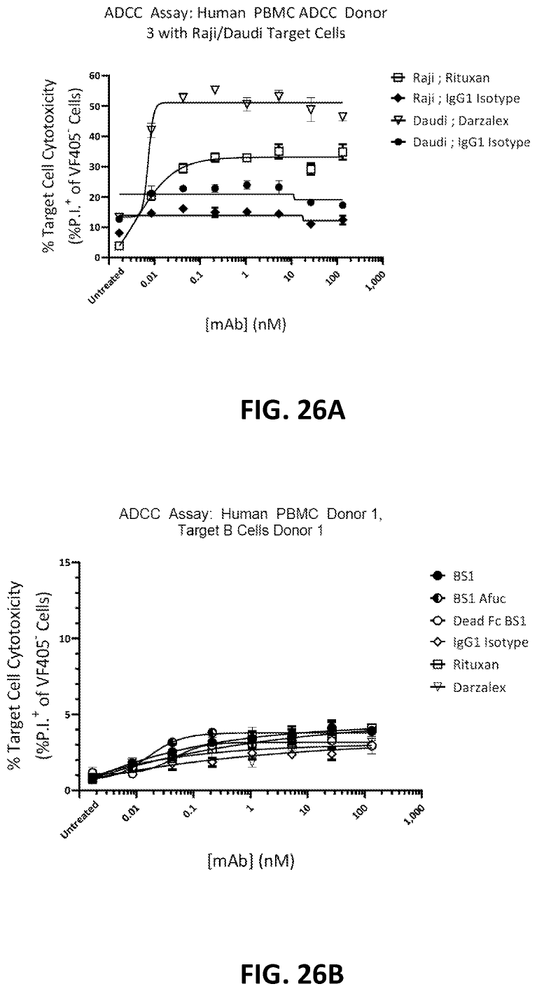

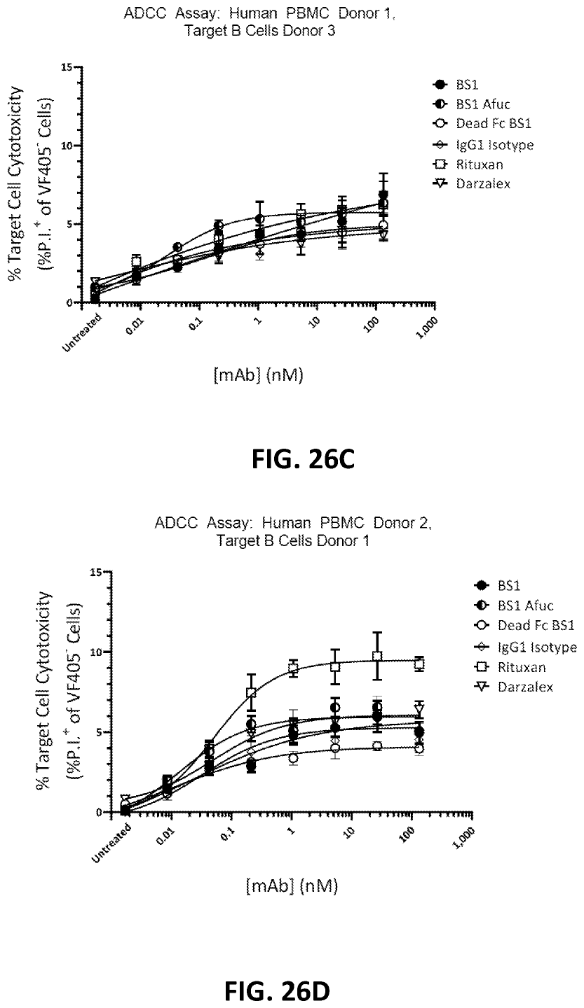

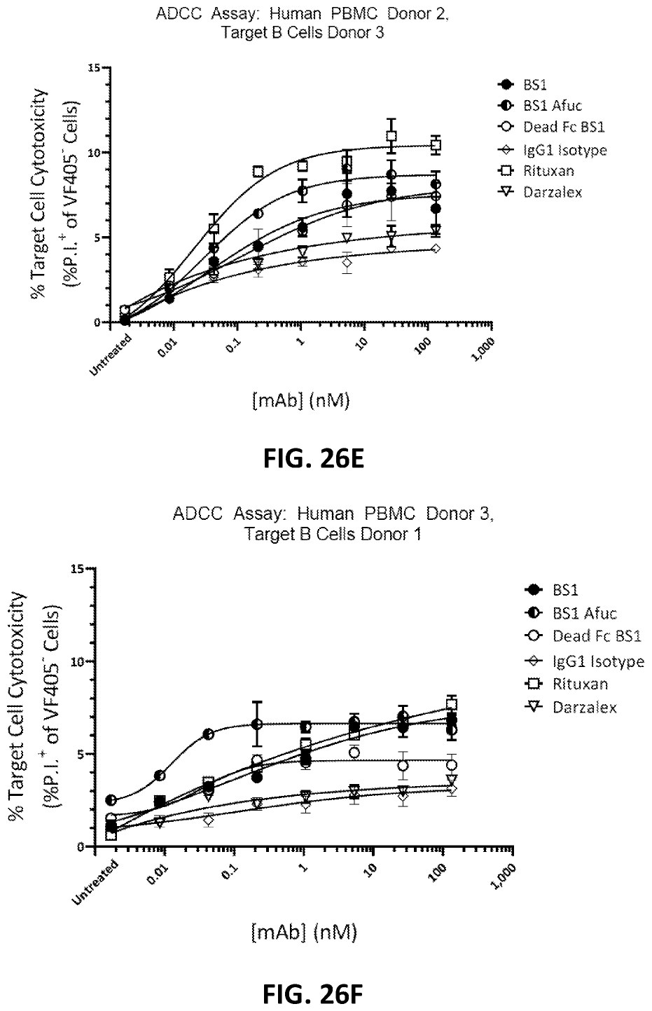

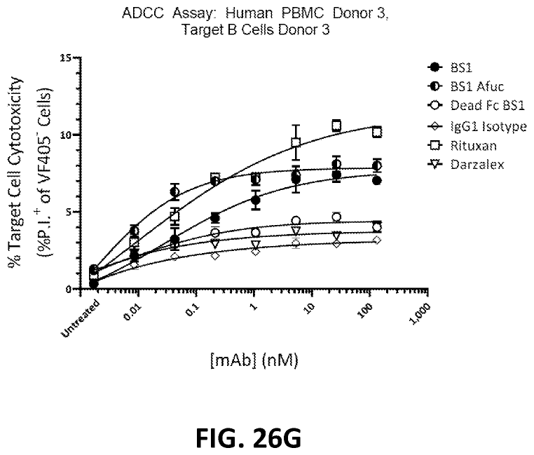

For cytotoxicity analysis, cells were stained with Propidium Iodide (P.I.) and analyzed by high-throughput flow cytometry. The percentage of P.I.+ cells within the VF405-population was analyzed as a measure of target cell cytotoxicity. FIG. 26A shows the level of ADCC on the Raji and Daudi control target cells using PMBCs from Donor 3. FIG. 26B shows the level of ADCC on target B cells from Donor 1 using PMBCs from Donor 1. FIG. 26C shows the level of ADCC on target B cells from Donor 3 using PMBCs from Donor 1. FIG. 26D shows the level of ADCC on target B cells from Donor 1 using PMBCs from Donor 2. FIG. 26E shows the level of ADCC on target B cells from Donor 3 using PMBCs from Donor 2. FIG. 26F shows the level of ADCC on target B cells from Donor 1 using PMBCs from Donor 3. FIG. 26G shows the level of ADCC on target B cells from Donor 3 using PMBCs from Donor 3. BS1 demonstrated a favorable ADCC profile (e.g., low ADCC) and it was therefore unexpected that the BS1 ADCC could be further reduced using a variant Fc (e.g., a “dead” Fc). Such further reductions could be advantageous in therapeutic treatment by lowering the possibility of immunological adverse events even lower. This is especially true for a possible mechanism of action where unwanted cells non-tumor cells (i.e., CD19×CD38 suppressive B cells) may be specifically targeted.

### Example 7: Cancer Patients Show Increased Immunosuppressive CD19 Positive, CD38 Positive CD20 Negative B Cells in Peripheral Blood and Tumor Samples

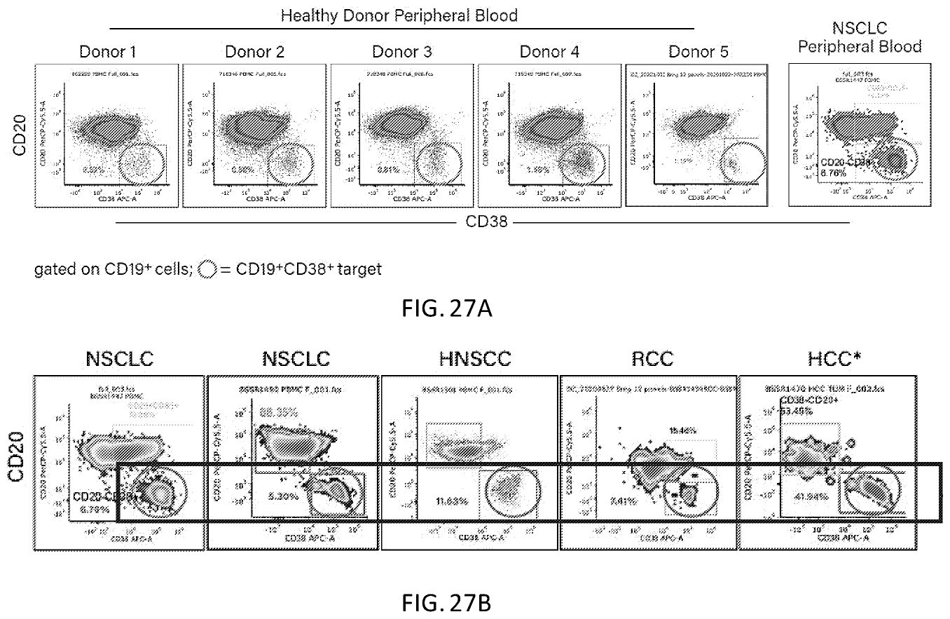

First the presence of CD19 positive, CD38 positive, CD20 low or negative B cells were assayed in the peripheral blood of healthy donors and cancer patients. As shown in FIG. 27A peripheral blood samples from patients with Non-small cell lung cancer showed increased amounts of CD19 positive, CD38 positive, CD20 negative B cells compared to healthy donors (e.g., 6.76% for NSCLC vs. 0.59% donor 1, 0.59% donor 2, 0.81% donor 3, 1.56% donor 4, and 1.19% donor 5). FIG. 27B shows that similar levels of CD19 positive, CD38 positive, CD20 negative or low B cells were observed in other cancers 6.76% and 5.30% for two different NSCL patients, 11.63% head and neck squamous cell carcinoma, 7.41% for renal cell carcinoma, and 41.94% for hepatocellular carcinoma (this last sample is from a tumor biopsy).

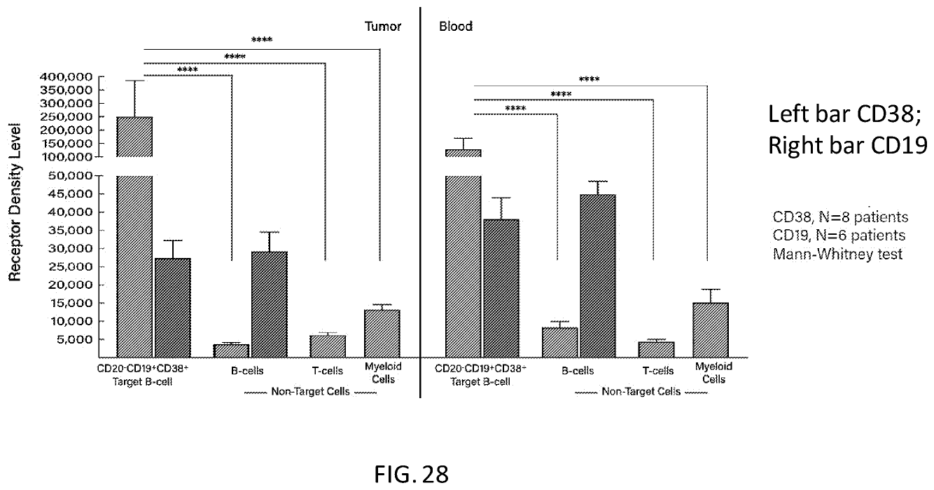

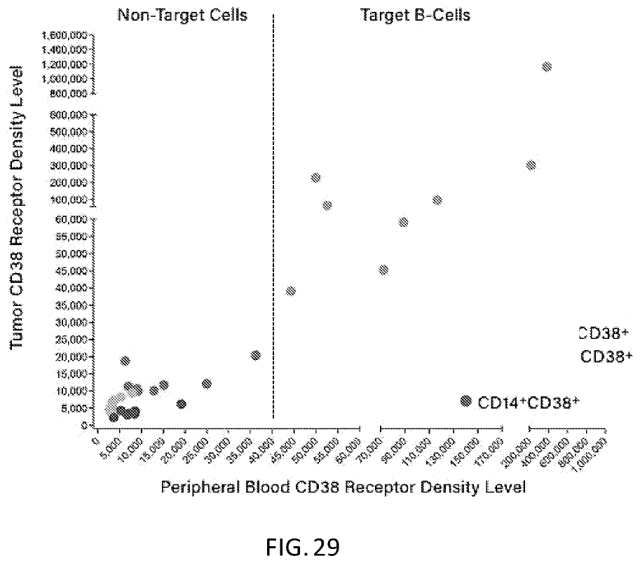

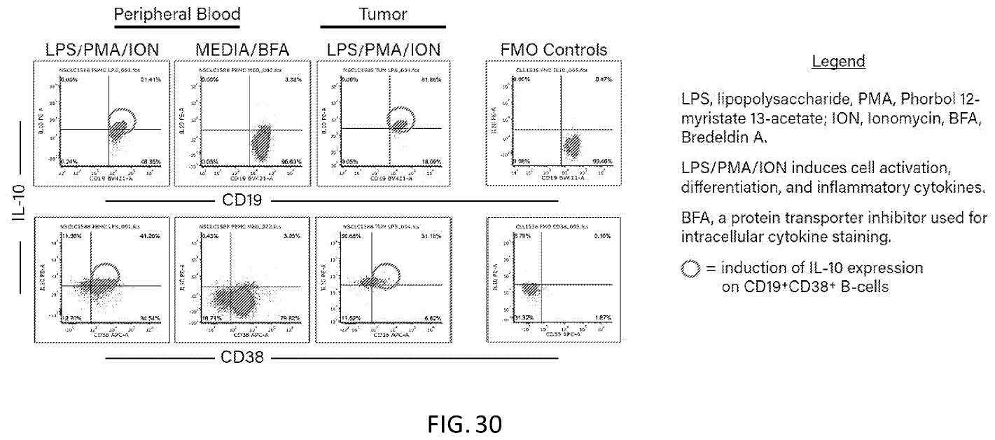

CD38 receptor density levels were measured in 8 patients with matched PBMCs and tumor samples (N=2 Renal Cell carcinoma; N=4 non-small cell lung cancer; N=2 head and neck squamous cell carcinoma), and in 6 of the 8 patients for CD19. FIG. 28 shows that CD38 was at least 10× more prevalent on CD20 negative, CD19 positive B cells in both tumors and peripheral blood compared to other cell-types such as T cells or myeloid cells. A receptor density of approximately 30,000 to 35,000 for CD38 was shown in the peripheral blood of cancer patients. FIG. 29 shows a strong correlation (Spearman's Rho=0.7870; p<0.0001 Mann-Whitney test) for CD38 receptor levels between tumor and peripheral blood. FIG. 30 shows that tumor infiltrating B cells and those in the periphery expressed high levels of the immune suppressive cytokine IL-10.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCES

| # | SEQUENCE | ANNOTATION |
|---|---|---|
| 1 | QVQLVQSGAEVKKPGSSVKVSCKASGGTFSSYTI | Anti-CD19_VH |
| | NWVRQAPGQGLEWMGGIIPIFGIPNYAQKFQGR | |
| | VTITADESTNTAYMELSSLRAEDTAVYYCARAS | |
| | GGSADYSYGMDVWGQGTAVTVSS | |
| 2 | DIQMTQSPSSLSASVGDRVTITCRASQGISSWLA | Anti-CD19_VL |
| | WYQQKPEKAPKSLIYAASSLQSGVPSRFSGSGSG | |
| | TDFTLTISSLQPEDFATYYCQQYKRYPYTFGQGT | |
| | KLEIK | |
| 3 | QVQLVQSGAEVKKPGSSVKVSCKASGGTFSSYA | Anti-CD38_VH |
| | FSWVRQAPGQGLEWMGRVIPFLGIANSAQKFQG | |
| | RVTITADKSTSTAYMDLSSLRSEDTAVYYCARD | |
| | DIAALGPFDYWGQGTLVTVSS | |
| 4 | DIQMTQSPSSLSASVGDRVTITCRASQGISSWLA | Anti-CD38_VL |
| | WYQQKPEKAPKSLIYAASSLQSGVPSRFSGSGSG | |
| | TDFTLTISSLQPEDFATYYCQQYNSYPRTFGQGT | |
| | KVEIK | |
| 5 | QVQLVQSGAEVKKPGSSVKVSCKASGGTFS | Anti-CD38_VH ver 2 |
| | SYAFSWVRQAPGQGLEWMGRVIPFLGIANS | |
| | AQKFQGRVTITADKSTSTAYMELSSLRSEDT | |
| | AVYYCARDDIAALGPFDYWGQGTLVTVSS | |
| 6 | QVQLVQSGAEVKKPGSSVKVSCKASGGTFS | Anti-CD19_Ver2 |
| | SYTINWVRQAPGQGLEWMGGIIPIFGIPNYA | |
| | QKFQGRVTITADESTNTAYMELSSLRSEDTA | |
| | VYYCARASGGSADYSYGMDVWGQGTLVT | |
| | VSS | |
| 7 | QVQLVQSGAEVKKPGSSVKVSCKASGGTFS | Anti-CD19_Ver 3 |
| | SYTINWVRQAPGQGLEWMGGIIPIFGIPNYA | |
| | QKFQGRVTITADESTNTAYMELSSLRSEDTA | |
| | VYYCARASGGSADYSYGMDVWGGGTLVT | |
| | VSS | |
| 11 | GGTFSSYT | Anti-CD19_VH_CDR1_IMGT |
| 12 | SYTIN | Anti-CD19_VH_CDR1_Kabat |
| 13 | GGTFSSY | Anti-CD19_VH_CDR1_Chothia |
| 14 | SSYTIN | Anti-CD19_VH_CDR1_Contact |
| 15 | GGTFSSYTIN | Anti-CD19_VH_CDR1_AbM |
| 21 | IIPIFGIP | Anti-CD19_VH_CDR2_IMGT |
| 22 | GIIPIFGIPNYAQKFQG | Anti-CD19_VH_CDR2_Kabat |
| 23 | PIFG | Anti-CD19_VH_CDR2_Chothia |
| 24 | WMGGIIPIFGIPN | Anti-CD19_VH_CDR2_Contact |
| 25 | GIIPIFGIPN | Anti-CD19_VH_CDR2_AbM |
| 31 | ARASGGSADYSYGMDV | Anti-CD19_VH_CDR3_IMGT |
| 32 | ASGGSADYSYGMDV | Anti-CD19_VH_CDR3_Kabat |
| 33 | SGGSADYSYGMD | Anti-CD19_VH_CDR3_Chothia |
| 34 | ARASGGSADYSYGMD | Anti-CD19_VH_CDR3_Contact |
| 35 | ASGGSADYSYGMDV | Anti-CD19_VH_CDR3_AbM |
| 41 | QGISSWLA | Anti-CD19_VL_CDR1_IMGT |
| 42 | RASQGISSWLA | Anti-CD19_VL_CDR1_Kabat |
| 43 | SQGISSW | Anti-CD19_VL_CDR1_Chothia |
| 44 | SSWLAWY | Anti-CD19_VL_CDR1_Contact |
| 45 | RASQGISSWLA | Anti-CD19_VL_CDR1_AbM |
| 51 | AAS | Anti-CD19_VL_CDR2_IMGT |
| 51 | AASSLQS | Anti-CD19_VL_CDR2_Kabat |
| 53 | AAS | Anti-CD19_VL_CDR2_Chothia |
| 54 | SLIYAASSLQ | Anti-CD19_VL_CDR2_Contact |
| 55 | AASSLQS | Anti-CD19_VL_CDR2_AbM |
| 61 | QQYKRYPYT | Anti-CD19_VL_CDR3_IMGT |
| 62 | QQYKRYPYT | Anti-CD19_VL_CDR3_Kabat |
| 63 | YKRYPY | Anti-CD19_VL_CDR3_Chothia |
| 64 | QQYKRYPY | Anti-CD19_VL_CDR3_Contact |
| 65 | QQYKRYPYT | Anti-CD19_VL_CDR3_AbM |
| 71 | GGTFSSYA | Anti-CD38_VH_CDR1_IMGT |
| 72 | SYAFS | Anti-CD38_VH_CDR1_Kabat |
| 73 | GGTFSSY | Anti-CD38_VH_CDR1_Chothia |
| 74 | SSYAFS | Anti-CD38_VH_CDR1_Contact |
| 75 | GGTFSSYAFS | Anti-CD38_VH_CDR1_AbM |
| 81 | VIPFLGIA | Anti-CD38_VH_CDR2_IMGT |
| 82 | RVIPFLGIANSAQKFQG | Anti-CD38_VH_CDR2_Kabat |
| 83 | PFLG | Anti-CD38_VH_CDR2_Chothia |
| 84 | WMGRVIPFLGIAN | Anti-CD38_VH_CDR2_Contact |
| 85 | RVIPFLGIAN | Anti-CD38_VH_CDR2_AbM |
| 91 | ARDDIAALGPFDY | Anti-CD38_VH_CDR3_IMGT |
| 92 | DDIAALGPFDY | Anti-CD38_VH_CDR3_Kabat |
| 93 | DIAALGPFD | Anti-CD38_VH_CDR3_Chothia |
| 94 | ARDDIAALGPFD | Anti-CD38_VH_CDR3_Contact |
| 95 | DDIAALGPFDY | Anti-CD38_VH_CDR3_AbM |
| 101 | QGISSWLA | Anti-CD38_VL_CDR1_IMGT |
| 102 | RASQGISSWLA | Anti-CD38_VL_CDR1_Kabat |
| 103 | SQGISSW | Anti-CD38_VL_CDR1_Chothia |
| 104 | SSWLAWY | Anti-CD38_VL_CDR1_Contact |
| 105 | RASQGISSWLA | Anti-CD38_VL_CDRI_AbM |
| 111 | AAS | Anti-CD38_VL_CDR2_IMGT |
| 112 | AASSLQS | Anti-CD38_VL_CDR2_Kabat |
| 113 | AAS | Anti-CD38_VL_CDR2_Chothia |
| 114 | SLIYAASSLQ | Anti-CD38_VL_CDR2_Contact |
| 115 | AASSLQS | Anti-CD38_VL_CDR2_AbM |
| 121 | QQYNSYPRT | Anti-CD38_VL_CDR3_IMGT |
| 122 | QQYNSYPRT | Anti-CD38_VL_CDR3_Kabat |
| 123 | YNSYPR | Anti-CD38_VH_CDR3_Chothia |
| 124 | QQYNSYPR | Anti-CD38_VH_CDR3_Contact |
| 125 | QQYNSYPRT | Anti-CD38_VH_CDR3_AbM |
| 150 | P-X1-L-G-X2-A; wherein X1 and | Anti-CD38_VH_CDR2 |
| | X2 is any amino acid | |
| 151 | PFLGTA | Anti-CD38 VH CDR2 |
| 152 | PHLGIA | Anti-CD38_VH_CDR2 |
| 153 | PHLGTA | Anti-CD38_VH_CDR2 |
| 154 | PQLGIA | Anti-CD38 VH CDR2 |
| 155 | PQLGTA | Anti-CD38_VH_CDR2 |
| 201 | QVQLVQSGAEVKKPGSSVKVSCKASGGTFSSYTI | BS1-19H |
| | NWVRQAPGQGLEWMGGIIPIFGIPNYAQKFQGR | |
| | VTITADESTNTAYMELSSLRAEDTAVYYCARAS | |
| | GGSADYSYGMDVWGQGTAVTVSSASTKGPSVF | |
| | PLAPSSKSTSGGTAALGCLVKDYFPEPVTVSWNS | |
| | GALTSGVHTFPAVLQSSGLYSLSSVVTVPSSSLG | |
| | TQTYICNVNHKPSNTKVDKKVEPKSCDKTHTCP | |
| | PCPAPELLGGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPAPIEKTISKAKGQPREPQVYTLPPSRDELTKN | |
| | QVSLSCAVKGFYPSDIAVEWESNGQPENNYKTT | |
| | PPVLDSDGSFFLVSKLTVDKSRWQQGNVFSCSV | |
| | MHEALHNHYTQKSLSLSPGK | |
| 202 | QVQLVQSGAEVKKPGSSVKVSCKASGGTFSSYA | BS1-38K |
| | FSWVRQAPGQGLEWMGRVIPFLGIANSAQKFQG | |
| | RVTITADKSTSTAYMDLSSLRSEDTAVYYCARD | |
| | DIAALGPFDYWGQGTLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQT | |
| | YICNVNHKPSNTKVDKKVEPKSCDKTHTCPPCP | |
| | APELLGGPSVFLFPPKPKDTLMISRTPEVTCVVV | |
| | DVSHEDPEVKFNWYVDGVEVHNAKTKPREEQY | |
| | NSTYRVVSVLTVLHQDWLNGKEYKCKVSNKAL | |
| | PAPIEKTISKAKGQPREPQVYTLPPSRDELTKNQV | |
| | SLWCLVKGFYPSDIAVEWESNGQPENNYKTTPP | |
| | VLDSDGSFFLYSKLTVDKSRWQQGNVFSCSVMH | |
| | EALHNHYTQKSLSLSPGK | |
| 203 | QVQLVQSGAEVKKPGSSVKVSCKASGGTFSSYTI | BS2-19H1 |
| | NWVRQAPGQGLEWMGGIIPIFGIPNYAQKFQGR | |
| | VTITADESTNTAYMELSSLRAEDTAVYYCARAS | |
| | GGSADYSYGMDVWGQGTAVTVSSGGGGSGGG | |
| | GSGGGGSDIQMTQSPSSLSASVGDRVTITCRASQ | |
| | GISSWLAWYQQKPEKAPKSLIYAASSLQSGVPSR | |
| | FSGSGSGTDFTLTISSLQPEDFATYYCQQYKRYP | |
| | YTFGQGTKLEIKAAEPKSSDKTHTCPPCPAPELL | |
| | GGPSVFLFPPKPKDTLMISRTPEVTCVVVDVSHE | |
| | DPEVKFNWYVDGVEVHNAKTKPREEQYNSTYR | |
| | VVSVLTVLHQDWLNGKEYKCKVSNKALPAPIEK | |
| | TISKAKGQPREPQVYTLPPSRDELTKNQVSLSCA | |
| | VKGFYPSDIAVEWESNGQPENNYKTTPPVLDSD | |
| | GSFFLVSKLTVDKSRWQQGNVFSCSVMHEALH | |
| | NHYTQKSLSLSPGK | |
| 204 | QVQLVQSGAEVKKPGSSVKVSCKASGGTFSSYTI | BS2-19H2 |
| | NWVRQAPGQGLEWMGGIIPIFGIPNYAQKFQGR | |
| | VTITADESTNTAYMELSSLRAEDTAVYYCARAS | |
| | GGSADYSYGMDVWGQGTAVTVSSGGGGSGGG | |
| | GSGGGGSDIQMTQSPSSLSASVGDRVTITCRASQ | |
| | GISSWLAWYQQKPEKAPKSLIYAASSLQSGVPSR | |
| | FSGSGSGTDFTLTISSLQPEDFATYYCQQYKRYP | |
| | YTFGQGTKLEIKGGGGDKTHTCPPCPAPELLGG | |
| | PSVFLFPPKPKDTLMISRTPEVTCVVVDVSHEDP | |
| | EVKFNWYVDGVEVHNAKTKPREEQYNSTYRVV | |
| | SVLTVLHQDWLNGKEYKCKVSNKALPAPIEKTI | |
| | SKAKGQPREPQVYTLPPSRDELTKNQVSLSCAV | |
| | KGFYPSDIAVEWESNGQPENNYKTTPPVLDSDG | |
| | SFFLVSKLTVDKSRWQQGNVFSCSVMHEALHN | |
| | HYTQKSLSLSPGK | |
| 205 | QVQLVQSGAEVKKPGSSVKVSCKASGGTFSSYA | BS2X-19H1 |
| | FSWVRQAPGQGLEWMGRVIPFLGIANSAQKFQG | |
| | RVTITADKSTSTAYMDLSSLRSEDTAVYYCARD | |
| | DIAALGPFDYWGQGTLVTVSSGGGGSGGGGSG | |
| | GGGSDIQMTQSPSSLSASVGDRVTITCRASQGISS | |
| | WLAWYQQKPEKAPKSLIYAASSLQSGVPSRFSG | |
| | SGSGTDFTLTISSLQPEDFATYYCQQYNSYPRTF | |
| | GQGTKVEIKAAEPKSSDKTHTCPPCPAPELLGGP | |
| | SVFLFPPKPKDTLMISRTPEVTCVVVDVSHEDPE | |
| | VKFNWYVDGVEVHNAKTKPREEQYNSTYRVVS | |
| | VLTVLHQDWLNGKEYKCKVSNKALPAPIEKTIS | |
| | KAKGQPREPQVYTLPPSRDELTKNQVSLWCLVK | |
| | GFYPSDIAVEWESNGQPENNYKTTPPVLDSDGSF | |
| | FLYSKLTVDKSRWQQGNVFSCSVMHEALHNHY | |
| | TQKSLSLSPGK | |
| 206 | QVQLVQSGAEVKKPGSSVKVSCKASGGTFSSYA | BS2X-19H2 |
| | FSWVRQAPGQGLEWMGRVIPFLGIANSAQKFQG | |
| | RVTITADKSTSTAYMDLSSLRSEDTAVYYCARD | |
| | DIAALGPFDYWGQGTLVTVSSGGGGSGGGGSG | |
| | GGGSDIQMTQSPSSLSASVGDRVTITCRASQGISS | |
| | WLAWYQQKPEKAPKSLIYAASSLQSGVPSRFSG | |
| | SGSGTDFTLTISSLQPEDFATYYCQQYNSYPRTF | |
| | GQGTKVEIKGGGGDKTHTCPPCPAPELLGGPSV | |
| | FLFPPKPKDTLMISRTPEVTCVVVDVSHEDPEVK | |
| | FNWYVDGVEVHNAKTKPREEQYNSTYRVVSVL | |
| | TVLHQDWLNGKEYKCKVSNKALPAPIEKTISKA | |
| | KGQPREPQVYTLPPSRDELTKNQVSLWCLVKGF | |
| | YPSDIAVEWESNGQPENNYKTTPPVLDSDGSFFL | |
| | YSKLTVDKSRWQQGNVFSCSVMHEALHNHYTQ | |
| | KSLSLSPGK | |
| 207 | QVQLVQSGAEVKKPGSSVKVSCKASGGTFSSYTI | BS3-19H38 |
| | NWVRQAPGQGLEWMGGIIPIFGIPNYAQKFQGR | |
| | VTITADESTNTAYMELSSLRAEDTAVYYCARAS | |
| | GGSADYSYGMDVWGQGTAVTVSSASTKGPSVF | |
| | PLAPSSKSTSGGTAALGCLVKDYFPEPVTVSWNS | |
| | GALTSGVHTFPAVLQSSGLYSLSSVVTVPSSSLG | |
| | TQTYICNVNHKPSNTKVDKKVEPKSCDKTHTCP | |
| | PCPAPELLGGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPAPIEKTISKAKGQPREPQVYTLPPSRDELTKN | |
| | QVSLWCLVKGFYPSDIAVEWESNGQPENNYKTT | |
| | PPVLDSDGSFFLYSKLTVDKSRWQQGNVFSCSV | |
| | MHEALHNHYTQKSLSLSPGGGGSGGGSQVQLV | |
| | QSGAEVKKPGSSVKVSCKASGGTFSSYAFSWVR | |
| | QAPGQGLEWMGRVIPFLGIANSAQKFQGRVTIT | |
| | ADKSTSTAYMDLSSLRSEDTAVYYCARDDIAAL | |
| | GPFDYWGQGTLVTVSSASTKGPSVFPLAPSSKST | |
| | SGGTAALGCLVKDYFPEPVTVSWNSGALTSGVH | |
| | TFPAVLQSSGLYSLSSVVTVPSSSLGTQTYICNVN | |
| | HKPSNTKVDKKVEPKSCDKTH | |
| 208 | DKTHTCPPCPAPELLGGPSVFLFPPKPKDTLMISR | BS3-Fc |
| | TPEVTCVVVDVSHEDPEVKFNWYVDGVEVHNA | |
| | KTKPREEQYNSTYRVVSVLTVLHQDWLNGKEY | |
| | KCKVSNKALPAPIEKTISKAKGQPREPQVYTLPP | |
| | SRDELTKNQVSLSCAVKGFYPSDIAVEWESNGQ | |
| | PENNYKTTPPVLDSDGSFFLVSKLTVDKSRWQQ | |
| | GNVFSCSVMHEALHNHYTQKSLSLSPGK | |
| 209 | QVQLVQSGAEVKKPGSSVKVSCKASGGTFSSYA | BS4-38K19 |
| | FSWVRQAPGQGLEWMGRVIPFLGIANSAQKFQG | |
| | RVTITADKSTSTAYMDLSSLRSEDTAVYYCARD | |
| | DIAALGPFDYWGQGTLVTVSSASTKGPSVFPLAP | |
| | SSKSTSGGTAALGCLVKDYFPEPVTVSWNSGAL | |
| | TSGVHTFPAVLQSSGLYSLSSVVTVPSSSLGTQT | |
| | YICNVNHKPSNTKVDKKVEPKSCDKTHTCPPCP | |
| | APELLGGPSVFLFPPKPKDTLMISRTPEVTCVVV | |
| | DVSHEDPEVKFNWYVDGVEVHNAKTKPREEQY | |
| | NSTYRVVSVLTVLHQDWLNGKEYKCKVSNKAL | |
| | PAPIEKTISKAKGQPREPQVYTLPPSRDELTKNQV | |
| | SLWCLVKGFYPSDIAVEWESNGQPENNYKTTPP | |
| | VLDSDGSFFLYSKLTVDKSRWQQGNVFSCSVMH | |
| | EALHNHYTQKSLSLSPGGGGSGGGSQVQLVQSG | |
| | AEVKKPGSSVKVSCKASGGTFSSYTINWVRQAP | |
| | GQGLEWMGGIIPIFGIPNYAQKFQGRVTITADEST | |
| | NTAYMELSSLRAEDTAVYYCARASGGSADYSY | |
| | GMDVWGQGTAVTVSSGGGGSGGGGSGGGGSDI | |
| | QMTQSPSSLSASVGDRVTITCRASQGISSWLAWY | |
| | QQKPEKAPKSLIYAASSLQSGVPSRFSGSGSGTD | |
| | FTLTISSLQPEDFATYYCQQYKRYPYTFGQGTKL | |
| | EIK | |
| 210 | DIQMTQSPSSLSASVGDRVTITCRASQGISSWLA | 19VL-CL |
| | WYQQKPEKAPKSLIYAASSLQSGVPSRFSGSGSG | |
| | TDFTLTISSLQPEDFATYYCQQYKRYPYTFGQGT | |
| | KLEIKRTVAAPSVFIFPPSDEQLKSGTASVVCLLN | |
| | NFYPREAKVQWKVDNALQSGNSQESVTEQDSK | |
| | DSTYSLSSTLTLSKADYEKHKVYACEVTHQGLS | |
| | SPVTKSFNRGEC | |
| 211 | | |
| 212 | QVQLVQSGAEVKKPGSSVKVSCKASGGTFS | 3C10 scFv-003Fab-Fc Knob |
| | SYTINWVRQAPGQGLEWMGGIIPIFGIPNYA | (BS3-19F38) |
| | QKFQGRVTITADESTNTAYMELSSLRAEDTA | |
| | VYYCARASGGSADYSYGMDVWGQGTAVT | |
| | VSSGGGGSGGGGSGGGGSDIQMTQSPSSLSA | |
| | SVGDRVTITCRASQGISSWLAWYQQKPEKA | |
| | PKSLIYAASSLQSGVPSRFSGSGSGTDFTLTIS | |
| | SLQPEDFATYYCQQYKRYPYTFGQGTKLEIK | |
| | GGGGGQVQLVQSGAEVKKPGSSVKVSCKA | |
| | SGGTFSSYTINWVRQAPGQGLEWMGGIIPIF | |
| | GIPNYAQKFQGRVTITADESTNTAYMELSSL | |
| | RAEDTAVYYCARASGGSADYSYGMDVWG | |
| | QGTAVTVSSASTKGPSVFPLAPSSKSTSGGT | |
| | AALGCLVKDYFPEPVTVSWNSGALTSGVHT | |
| | FPAVLQSSGLYSLSSVVTVPSSSLGTQTYICN | |
| | VNHKPSNTKVDKKVEPKSCDKTHTCPPCPA | |
| | PELLGGPSVFLFPPKPKDTLMISRTPEVTCVV | |
| | VDVSHEDPEVKFNWYVDGVEVHNAKTKPR | |
| | EEQYNSTYRVVSVLTVLHQDWLNGKEYKC | |
| | KVSNKALPAPIEKTISKAKGQPREPQVYTLPP | |
| | SRDELTKNQVSLWCLVKGFYPSDIAVEWES | |
| | NGQPENNYKTTPPVLDSDGSFFLYSKLTVDK | |
| | SRWQQGNVFSCSVMHEALHNHYTQKSLSLS | |
| | PGK | |
| 213 | DIQMTQSPSSLSASVGDRVTITCRASQGISSW | 003 VL (38VL-CL) |
| | LAWYQQKPEKAPKSLIYAASSLQSGVPSRFS | |
| | GSGSGTDFTLTISSLQPEDFATYYCQQYNSY | |
| | PRTFGQGTKVEIKRTVAAPSVFIFPPSDEQLK | |
| | SGTASVVCLLNNFYPREAKVQWKVDNALQ | |
| | SGNSQESVTEQDSKDSTYSLSSTLTLSKADY | |
| | EKHKVYACEVTHQGLSSPVTKSFNRGEC | |
| 214 | DKTHTCPPCPAPELLGGPSVFLFPPKPKDTL | Fc Hole (BS3-Fc) |
| | MISRTPEVTCVVVDVSHEDPEVKFNWYVDG | |
| | VEVHNAKTKPREEQYNSTYRVVSVLTVLHQ | |
| | DWLNGKEYKCKVSNKALPAPIEKTISKAKG | |
| | QPREPQVYTLPPSRDELTKNQVSLSCAVKGF | |
| | YPSDIAVEWESNGQPENNYKTTPPVLDSDGS | |
| | FFLVSKLTVDKSRWQQGNVFSCSVMHEALH | |
| | NHYTQKSLSLSPGK | |
| 215 | QVQLVQSGAEVKKPGSSVKVSCKASGGTFS | BS1-38K ver2 |
| | SYAFSWVRQAPGQGLEWMGRVIPFLGIANS | |
| | AQKFQGRVTITADKSTSTAYMELSSLRSEDT | |
| | AVYYCARDDIAALGPFDYWGQGTLVTVSSA | |
| | STKGPSVFPLAPSSKSTSGGTAALGCLVKDY | |
| | FPEPVTVSWNSGALTSGVHTFPAVLQSSGLY | |
| | SLSSVVTVPSSSLGTQTYICNVNHKPSNTKV | |
| | DKKVEPKSCDKTHTCPPCPAPELLGGPSVFL | |
| | FPPKPKDTLMISRTPEVTCVVVDVSHEDPEV | |
| | KFNWYVDGVEVHNAKTKPREEQYNSTYRV | |
| | VSVLTVLHQDWLNGKEYKCKVSNKALPAPI | |
| | EKTISKAKGQPREPQVYTLPPSRDELTKNQV | |
| | SLWCLVKGFYPSDIAVEWESNGQPENNYKT | |
| | TPPVLDSDGSFFLYSKLTVDKSRWQQGNVFS | |
| | CSVMHEALHNHYTQKSLSLSPGK | |
| 216 | QVQLVQSGAEVKKPGSSVKVSCKASGGTFS | BS1-19H ver2 |
| | SYTINWVRQAPGQGLEWMGGIIPIFGIPNYA | |
| | QKFQGRVTITADESTNTAYMELSSLRSEDTA | |
| | VYYCARASGGSADYSYGMDVWGQGTLVT | |
| | VSSASTKGPSVFPLAPSSKSTSGGTAALGCL | |
| | VKDYFPEPVTVSWNSGALTSGVHTFPAVLQ | |
| | SSGLYSLSSVVTVPSSSLGTQTYICNVNHKPS | |
| | NTKVDKKVEPKSCDKTHTCPPCPAPELLGGP | |
| | SVFLFPPKPKDTLMISRTPEVTCVVVDVSHE | |
| | DPEVKFNWYVDGVEVHNAKTKPREEQYNS | |
| | TYRVVSVLTVLHQDWLNGKEYKCKVSNKA | |
| | LPAPIEKTISKAKGQPREPQVYTLPPSRDELT | |
| | KNQVSLSCAVKGFYPSDIAVEWESNGQPEN | |
| | NYKTTPPVLDSDGSFFLVSKLTVDKSRWQQ | |
| | GNVFSCSVMHEALHNHYTQKSLSLSPGK | |
| 217 | QVQLVQSGAEVKKPGSSVKVSCKASGGTFS | BS2-19H ver2 |
| | SYTINWVRQAPGQGLEWMGGIIPIFGIPNYA | |
| | QKFQGRVTITADESTNTAYMELSSLRSEDTA | |
| | VYYCARASGGSADYSYGMDVWGGGTLVT | |
| | VSSGGGGSGGGGSGGGGSDIQMTQSPSSLSA | |
| | SVGDRVTITCRASQGISSWLAWYQQKPEKA | |
| | PKSLIYAASSLQSGVPSRFSGSGSGTDFTLTIS | |
| | SLQPEDFATYYCQQYKRYPYTFGQGTKLEIK | |
| | AAEPKSSDKTHTCPPCPAPELLGGPSVFLFPP | |
| | KPKDTLMISRTPEVTCVVVDVSHEDPEVKFN | |
| | WYVDGVEVHNAKTKPREEQYNSTYRVVSV | |
| | LTVLHQDWLNGKEYKCKVSNKALPAPIEKT | |
| | ISKAKGQPREPQVYTLPPSRDELTKNQVSLS | |
| | CAVKGFYPSDIAVEWESNGQPENNYKTTPP | |
| | VLDSDGSFFLVSKLTVDKSRWQQGNVFSCS | |
| | VMHEALHNHYTQKSLSLSPGK | |
| 218 | QVQLVQSGAEVKKPGSSVKVSCKASGGTFS | BS1M-1 |
| | SYAFSWVRQAPGQGLEWMGRVIPFLGTANS | |
| | AQKFQGRVTITADKSTSTAYMELSSLRSEDT | |
| | AVYYCARDDIAALGPFDYWGQGTLVTVSS | |
| 219 | QVQLVQSGAEVKKPGSSVKVSCKASGGTFS | BS1M-3 |
| | SYAFSWVRQAPGQGLEWMGRVIPHLGIANS | |
| | AQKFQGRVTITADKSTSTAYMELSSLRSEDT | |
| | AVYYCARDDIAALGPFDYWGQGTLVTVSS | |
| 220 | QVQLVQSGAEVKKPGSSVKVSCKASGGTFS | BS1M-4 |
| | SYAFSWVRQAPGQGLEWMGRVIPHLGTANS | |
| | AQKFQGRVTITADKSTSTAYMELSSLRSEDT | |
| | AVYYCARDDIAALGPFDYWGQGTLVTVSS | |
| 221 | QVQLVQSGAEVKKPGSSVKVSCKASGGTFS | BS1M-6 |
| | SYAFSWVRQAPGQGLEWMGRVIPQLGIANS | |
| | AQKFQGRVTITADKSTSTAYMELSSLRSEDT | |
| | AVYYCARDDIAALGPFDYWGQGTLVTVSS | |
| 222 | QVQLVQSGAEVKKPGSSVKVSCKASGGTFS | BS1M-7 |
| | SYAFSWVRQAPGQGLEWMGRVIPQLGTANS | |
| | AQKFQGRVTITADKSTSTAYMELSSLRSEDT | |
| | AVYYCARDDIAALGPFDYWGQGTLVTVSS | |
| 223 | DIQMTQSPSSLSASVGDRVTITCRASQGISSH | BSM-10 |
| | LAWYQQKPEKAPKSLIYAASSLQSGVPSRFS | |
| | GSGSGTDFTLTISSLQPEDFATYYCQQYNSY | |
| | PRTFGQGTKVEIK | |
| 301 | QVQLVQSGAEVKKPGSSVKVSCKASGGTFSSYTINWV | BS1-19H-dFc |
| | RQAPGQGLEWMGGIIPIFGIPNYAQKFQGRVTITADES | |
| | TNTAYMELSSLRAEDTAVYYCARASGGSADYSYGMDV | |
| | WGQGTAVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEPK | |
| | SCDKTHTCPPCPAPEAEGAPSVFLFPPKPKDTLMISRT | |
| | PEVTCVVVDVSHEDPEVKFNWYVDGVEVHNAKTKPR | |
| | EEQYNSTYRVVSVLTVLHQDWLNGKEYKCKVSNKAL | |
| | PSSIEKTISKAKGQPREPQVYTLPPSRDELTKNQVSLSC | |
| | AVKGFYPSDIAVEWESNGQPENNYKTTPPVLDSDGSF | |
| | FLVSKLTVDKSRWQQGNVFSCSVMHEALHNHYTQKS | |
| | LSLSPGK | |
| 302 | QVQLVQSGAEVKKPGSSVKVSCKASGGTFSSYAFSWV | BS1-38K-dFc |
| | RQAPGQGLEWMGRVIPFLGIANSAQKFQGRVTITADK | |
| | STSTAYMDLSSLRSEDTAVYYCARDDIAALGPFDYWG | |
| | QGTLVTVSSASTKGPSVFPLAPSSKSTSGGTAALGCLV | |
| | KDYFPEPVTVSWNSGALTSGVHTFPAVLQSSGLYSLSS | |
| | VVTVPSSSLGTQTYICNVNHKPSNTKVDKKVEPKSCD | |
| | KTHTCPPCPAPEAEGAPSVFLFPPKPKDTLMISRTPEV | |
| | TCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 303 | QVQLVQSGAEVKKPGSSVKVSCKASGGTFSSYAFSWV | BS1-38K-dFc ver2 |
| | RQAPGQGLEWMGRVIPFLGIANSAQKFQGRVTITADK | |
| | STSTAYMELSSLRSEDTAVYYCARDDIAALGPFDYWG | |
| | QGTLVTVSSASTKGPSVFPLAPSSKSTSGGTAALGCLV | |
| | KDYFPEPVTVSWNSGALTSGVHTFPAVLQSSGLYSLSS | |
| | VVTVPSSSLGTQTYICNVNHKPSNTKVDKKVEPKSCD | |
| | KTHTCPPCPAPEAEGAPSVFLFPPKPKDTLMISRTPEV | |
| | TCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 304 | QVQLVQSGAEVKKPGSSVKVSCKASGGTFSSYTINWV | BS1-19H-dFc ver2 |
| | RQAPGQGLEWMGGIIPIFGIPNYAQKFQGRVTITADES | |
| | TNTAYMELSSLRSEDTAVYYCARASGGSADYSYGMDV | |
| | WGQGTLVTVSSASTKGPSVFPLAPSSKSTSGGTAALGC | |
| | LVKDYFPEPVTVSWNSGALTSGVHTFPAVLQSSGLYS | |
| | LSSVVTVPSSSLGTQTYICNVNHKPSNTKVDKKVEPKS | |
| | CDKTHTCPPCPAPEAEGAPSVFLFPPKPKDTLMISRTP | |
| | EVTCVVVDVSHEDPEVKFNWYVDGVEVHNAKTKPRE | |
| | EQYNSTYRVVSVLTVLHQDWLNGKEYKCKVSNKALP | |
| | SSIEKTISKAKGQPREPQVYTLPPSRDELTKNQVSLSC | |
| | AVKGFYPSDIAVEWESNGQPENNYKTTPPVLDSDGSF | |
| | FLVSKLTVDKSRWQQGNVFSCSVMHEALHNHYTQKS | |
| | LSLSPGK | |
| 305 | QVQLVQSGAEVKKPGSSVKVSCKASGGTFSSYAFSWV | BS1M-1-BS1-38K-dFc |
| | RQAPGQGLEWMGRVIPFLGTANSAQKFQGRVTITAD | |
| | KSTSTAYMDLSSLRSEDTAVYYCARDDIAALGPFDYW | |
| | GQGTLVTVSSASTKGPSVFPLAPSSKSTSGGTAALGCL | |
| | VKDYFPEPVTVSWNSGALTSGVHTFPAVLQSSGLYSL | |
| | SSVVTVPSSSLGTQTYICNVNHKPSNTKVDKKVEPKSC | |
| | DKTHTCPPCPAPEAEGAPSVFLFPPKPKDTLMISRTPE | |
| | VTCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 306 | QVQLVQSGAEVKKPGSSVKVSCKASGGTFSSYAFSWV | BS1M-3-BS1-38K-dFc |
| | RQAPGQGLEWMGRVIPHLGIANSAQKFQGRVTITAD | |
| | KSTSTAYMDLSSLRSEDTAVYYCARDDIAALGPFDYW | |
| | GQGTLVTVSSASTKGPSVFPLAPSSKSTSGGTAALGCL | |
| | VKDYFPEPVTVSWNSGALTSGVHTFPAVLQSSGLYSL | |
| | SSVVTVPSSSLGTQTYICNVNHKPSNTKVDKKVEPKSC | |
| | DKTHTCPPCPAPEAEGAPSVFLFPPKPKDTLMISRTPE | |
| | VTCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 307 | QVQLVQSGAEVKKPGSSVKVSCKASGGTFSSYAFSWV | BS1M-4-BS1-38K-dFc |
| | RQAPGQGLEWMGRVIPQLGIANSAQKFQGRVTITADK | |
| | STSTAYMDLSSLRSEDTAVYYCARDDIAALGPFDYWG | |
| | QGTLVTVSSASTKGPSVFPLAPSSKSTSGGTAALGCLV | |
| | KDYFPEPVTVSWNSGALTSGVHTFPAVLQSSGLYSLSS | |
| | VVTVPSSSLGTQTYICNVNHKPSNTKVDKKVEPKSCD | |
| | KTHTCPPCPAPEAEGAPSVFLFPPKPKDTLMISRTPEV | |
| | TCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 308 | QVQLVQSGAEVKKPGSSVKVSCKASGGTFSSYAFSWV | BS1M-1-BS1-38K-dFc-ver2 |
| | RQAPGQGLEWMGRVIPFLGTANSAQKFQGRVTITAD | |
| | KSTSTAYMELSSLRSEDTAVYYCARDDIAALGPFDYW | |
| | GQGTLVTVSSASTKGPSVFPLAPSSKSTSGGTAALGCL | |
| | VKDYFPEPVTVSWNSGALTSGVHTFPAVLQSSGLYSL | |
| | SSVVTVPSSSLGTQTYICNVNHKPSNTKVDKKVEPKSC | |
| | DKTHTCPPCPAPEAEGAPSVFLFPPKPKDTLMISRTPE | |
| | VTCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 309 | QVQLVQSGAEVKKPGSSVKVSCKASGGTFSSYAFSWV | BS1M-3-BS1-38K-dFc-ver2 |
| | RQAPGQGLEWMGRVIPHLGIANSAQKFQGRVTITAD | |
| | KSTSTAYMELSSLRSEDTAVYYCARDDIAALGPFDYW | |
| | GQGTLVTVSSASTKGPSVFPLAPSSKSTSGGTAALGCL | |
| | VKDYFPEPVTVSWNSGALTSGVHTFPAVLQSSGLYSL | |
| | SSVVTVPSSSLGTQTYICNVNHKPSNTKVDKKVEPKSC | |
| | DKTHTCPPCPAPEAEGAPSVFLFPPKPKDTLMISRTPE | |
| | VTCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 310 | QVQLVQSGAEVKKPGSSVKVSCKASGGTFSSYAFSWV | BS1M-4-BS1-38K-dFc-ver2 |
| | RQAPGQGLEWMGRVIPQLGIANSAQKFQGRVTITADK | |
| | STSTAYMELSSLRSEDTAVYYCARDDIAALGPFDYWG | |
| | QGTLVTVSSASTKGPSVFPLAPSSKSTSGGTAALGCLV | |
| | KDYFPEPVTVSWNSGALTSGVHTFPAVLQSSGLYSLSS | |
| | VVTVPSSSLGTQTYICNVNHKPSNTKVDKKVEPKSCD | |
| | KTHTCPPCPAPEAEGAPSVFLFPPKPKDTLMISRTPEV | |
| | TCVVVDVSHEDPEVKFNWYVDGVEVHNAKTKPREE | |
| | QYNSTYRVVSVLTVLHQDWLNGKEYKCKVSNKALPS | |
| | SIEKTISKAKGQPREPQVYTLPPSRDELTKNQVSLWCL | |
| | VKGFYPSDIAVEWESNGQPENNYKTTPPVLDSDGSFF | |
| | LYSKLTVDKSRWQQGNVFSCSVMHEALHNHYTQKSL | |
| | SLSPGK | |
| 311 | ASTKGPSVFPLAPSSKSTSGGTAALGCLVKDYFPEPVT | dFc |
| | VSWNSGALTSGVHTFPAVLQSSGLYSLSSVVTVPSSSL | |
| | GTQTYICNVNHKPSNTKVDKKVEPKSCDKTHTCPPCP | |
| | APEAEGAPSVFLFPPKPKDTLMISRTPEVTCVVVDVS | |
| | HEDPEVKFNWYVDGVEVHNAKTKPREEQYNSTYRV | |
| | VSVLTVLHQDWLNGKEYKCKVSNKALPSSIEKTISKA | |
| | KGQPREPQVYTLPPSRDELTKNQVSLSCAVKGFYPSDI | |
| | AVEWESNGQPENNYKTTPPVLDSDGSFFLVSKLTVDK | |
| | SRWQQGNVFSCSVMHEALHNHYTQKSLSLSPGK | |

1. A method of treating a cancer or tumor associated with CD19 positive, CD38 high immunosuppressive B cells in an individual comprising administering to the individual a bispecific antibody that binds CD19 and CD38, thereby treating the cancer or tumor associated with CD19 positive, CD38 high immunosuppressive B cells.

2. The method of claim 1, wherein the bispecific antibody comprises a variant Fc region comprising one or more mutations relative to a wildtype Fc region, wherein the variant Fc region exhibits altered effector function compared to the wildtype Fc region.

3. The method of claim 2, wherein the reduced effector function is selected from the list consisting of reduced antibody-dependent cell-mediated cytotoxicity (ADCC), reduced complement mediated cytotoxicity (CDC), reduced affinity for C1q, and any combination thereof.

4. The method of any one of claim 2 or 3, wherein the variant Fc region comprises IgG1 Fc region, and wherein the one or more mutations comprise: (a) 297A, 297Q, 297G, or 297D, (b) 279F, 279K, or 279L, (c) 228P, (d) 235A, 235E, 235G, 235Q, 235R, or 235S, (e) 237A, 237E, 237K, 237N, or 237R, (f) 234A, 234V, or 234F, (g) 233P, (h) 328A, (i) 327Q or 327T, (j) 329A, 329G, 329Y, or 329R (k) 331S, (1) 236F or 236R, (m) 238A, 238E, 238G, 238H, 238I, 238V, 238W, or 238Y, (n) 248A, (o) 254D, 254E, 254G, 254H, 254I, 254N, 254P, 254Q, 254T, or 254V, (p) 255N, (q) 256H, 256K, 256R, or 256V, (r) 264S, (s) 265H, 265K, 265S, 265Y, or 265A, (t) 267G, 267H, 267I, or 267K, (u) 268K, (v) 269N or 269Q, (w) 270A, 270G, 270M, or 270N, (x) 271T, (y) 272N, (z) 292E, 292F, 292G, or 292I, (aa) 293S, (bb) 301W, (cc) 304E, (dd) 311E, 311G, or 311S, (ee) 316F, (ff) 328V, (gg) 330R, (hh) 339E or 339L, (ii) 343I or 343V, (jj) 373A, 373G, or 373S, (kk) 376E, 376W, or 376Y, (ll) 380D, (mm) 382D or 382P, (nn) 385P, (00) 424H, 424M, or 424V, (pp) 434I, (qq) 438G, (rr) 439E, 439H, or 439Q, (ss) 440A, 440D, 440E, 440F, 440M, 440T, or 440V, (tt) K322A, (uu) L235E, (vv) L234A and L235A, (ww) L234A, L235A, and G237A, (xx) L234A, L235A, and P329G, (yy) L234F, L235E, and P331S, (zz) L234A, L235E, and G237A, (aaa), L234A, L235E, G237A, and P331S (bbb) L234A, L235A, G237A, P238S, H268A, A330S, and P331S, (ccc) L234A, L235A, and P329A, (ddd) G236R and L328R, (eee) G237A, (fff) F241A, (ggg) V264A, (hhh) D265A, (iii) D265A and N297A, (jjj) D265A and N297G, (kkk) D270A, (lll) A330L, (mmm) P331A or P331S, or (nnn) E233P, (000) L234A, L235E, G237A, A330S, and P331S or (ppp) any combination of (a)-(uu), per EU numbering.

5. The method of any one of claim 2 or 3, wherein the variant Fc region is selected from Table 1.

6. The method of any one of claims 2 to 5, wherein the one or more mutations relative to a wildtype Fc region comprises one or more substitutions at L234, L235, G237, A330, or P331 by EU Numbering.

7. The method of any one of claims 2 to 5, wherein the one or more mutations relative to a wildtype Fc region comprises L234A, L235E, G237A, A330S, or P331S by EU Numbering.

8. The method of any one of claims 2 to 5, wherein the one or more mutations relative to a wildtype Fc region comprises K322A by EU Numbering.

9. The method of any one of claims 2 to 5, wherein the one or more mutations relative to a wildtype Fc region consists of K322A by EU Numbering.

10. The method of any one of claims 2 to 5, wherein the one or more mutations relative to a wildtype Fc region comprises or consists of S329D and 1332E by EU numbering.

11. The method of any one of claims 2 to 5, wherein the one or more mutations relative to a wildtype Fc region comprises or consists of L234A, L235E, G237A, A330S, and P331S by EU numbering.

12. The method of any one of claims 2 to 5, wherein the one or more mutations relative to a wildtype Fc region comprises or consists of L234A, L235A, and P329G by EU numbering.

13. The method of any one of claims 2 to 5, wherein the one or more mutations relative to a wildtype Fc region is selected from the group consisting of:
N297A/Q/G; L235A/G237A/E318A; L234A/L235A; G236R/L328R; S298G/T299A;
L234F/L235E/P331S; H268Q/V309L/A330S/P331S; L234A/L235A/P329G;
V234A/G237A/P238S/H268A/V309L/A330S/P331S; and L234F/L235E/D265A.

14. The method of any one of claims 1 to 13, wherein the bispecific antibody that binds CD19 and CD38 comprises a CD38 antigen binding component that comprises:
a) a heavy chain complementarity determining region 1 (HCDR1) comprising an amino acid sequence set forth in any one of SEQ ID NOs: 71-75;
b) a heavy chain complementarity determining region 2 (HCDR2) comprising an amino acid sequence set forth in any one of SEQ ID NOs: 81-85, or 151-155;
c) a heavy chain complementarity determining region 3 (HCDR3) comprising an amino acid sequence set forth in any one of SEQ ID NOs: 91-95;
d) a light chain complementarity determining region 1 (LCDR1) comprising an amino acid sequence set forth in any one of SEQ ID NOs: 101-105;
e) a light chain complementarity determining region 2 (LCDR2) comprising an amino acid sequence set forth in any one of SEQ ID NOs: 111-115; and
f) a light chain complementarity determining region 3 (LCDR3) comprising an amino acid sequence set forth in any one of SEQ ID NOs: 121-125; and wherein a CD19 antigen binding component comprises:
g) a heavy chain complementarity determining region 1 (HCDR1) comprising an amino acid sequence set forth in any one of SEQ ID NOs: 11-15;
h) a heavy chain complementarity determining region 2 (HCDR2) comprising an amino acid sequence set forth in any one of SEQ ID NOs: 21-25;
i) a heavy chain complementarity determining region 3 (HCDR3) comprising an amino acid sequence set forth in any one of SEQ ID NOs: 31-35;
j) a light chain complementarity determining region 1 (LCDR1) comprising an amino acid sequence set forth in any one of SEQ ID NOs: 101-105;
k) a light chain complementarity determining region 2 (LCDR2) comprising an amino acid sequence set forth in any one of SEQ ID NOs: 111-115; and
l) a light chain complementarity determining region 3 (LCDR3) comprising an amino acid sequence set forth in any one of SEQ ID NOs: 121-125.

15. The method of claim 14, wherein the CD38 antigen binding component comprises an HCDR2 amino acid sequence comprising the amino acid sequence set forth in SEQ ID NO: 151 to 155.

16. The method of claim 14, wherein the CD38 antigen binding component comprises an HCDR2 amino acid sequence comprising the amino acid sequence set forth in SEQ ID NO: 154.

17. The method of any one of claims 1 to 16, wherein the bispecific antibody that binds CD19 and CD38 comprises a CD38 antigen binding component comprises an HCDR2 amino acid sequence comprising any one of the amino acid sequences set forth in SEQ ID NO: 81 to 85.

18. The method of claims 1 to 17, wherein the bispecific antibody that binds CD19 and CD38 comprises an anti-CD38 immunoglobulin heavy chain variable region that comprises an amino acid sequence having at least about 90% identity to SEQ ID NO: 3 or 5; and the anti-CD38 immunoglobulin light chain variable region comprises an amino acid sequence having at least about 90% identity to SEQ ID NO: 4.

19. The method of claim 18, wherein the bispecific antibody that binds CD19 and CD38 comprises an anti-CD38 immunoglobulin heavy chain variable region that comprises an amino acid sequence identical to SEQ ID NO: 3 or 5; and the anti-CD38 immunoglobulin light chain variable region comprises an amino acid sequence identical to SEQ ID NO: 4.

20. The method of any one of claims 1 to 19, wherein the bispecific antibody that binds CD19 and CD38 comprises an anti-CD19 immunoglobulin heavy chain variable region that comprises an amino acid sequence having at least about 90% identity to SEQ ID NO: 1, 6, or 7; and the anti-CD19 immunoglobulin light chain variable region comprises an amino acid sequence having at least about 90% identity to SEQ ID NO: 2.

21. The method of claim 20, wherein the bispecific antibody that binds CD19 and CD38 comprises an anti-CD19 immunoglobulin heavy chain variable region that comprises an amino acid sequence identical to SEQ ID NO: 1, 6, or 7; and the anti-CD19 immunoglobulin light chain variable region comprises an amino acid sequence identical to SEQ ID NO: 2.

22. The method of any one of claims 1 to 21, wherein the bispecific antibody that binds CD19 and CD38 comprises a anti-CD38 immunoglobulin heavy chain variable region further comprises an immunoglobulin heavy chain constant region, wherein the anti-CD38 immunoglobulin heavy chain constant region comprises one or more amino acid substitutions that disfavors homodimerization of the anti-CD38 immunoglobulin heavy chain constant region and promotes heterodimerization of the anti-CD38 immunoglobulin heavy chain constant region with a non-anti-CD38 immunoglobulin heavy chain constant region.

23. The method of claim 22, wherein the bispecific antibody that binds CD19 and CD38 comprises an anti-CD38 immunoglobulin heavy chain constant region comprises a T366W substitution (EU numbering) or T366S/L368A/Y407V substitution (EU numbering), such that the heterodimerization of the anti-CD38 immunoglobulin heavy chain constant region and the non-anti-CD38 immunoglobulin heavy chain constant region is favored compared to homodimerization of the anti-CD38 immunoglobulin heavy chain.

24. The method of any one of claims 1 to 23, wherein the bispecific antibody comprises an anti-CD19 immunoglobulin heavy chain variable region further comprises an immunoglobulin heavy chain constant region, wherein the anti-CD19 immunoglobulin heavy chain constant region comprises one or more amino acid substitutions that disfavors homodimerization of the anti-CD19 immunoglobulin heavy chain constant region and promotes heterodimerization of the second heavy chain constant region with a non-anti-CD19 immunoglobulin heavy chain constant region.

25. The method of claim 24, wherein the anti-CD19 immunoglobulin heavy chain constant region comprises a T366W substitution (EU numbering) or a T366S/L368A/Y407V substitution (EU numbering), such that the heterodimerization of the anti-CD19 immunoglobulin heavy chain constant region and the non-anti-CD19 immunoglobulin heavy chain immunoglobulin heavy chain constant region is favored compared to homodimerization of the anti-CD19 immunoglobulin heavy chain.

26. The method of any one of claims 1 to 25, wherein the anti-CD38 immunoglobulin light chain variable region further comprises an immunoglobulin light chain constant region.

27. The method of any one of claims 1 to 25, wherein the bispecific antibody that binds CD19 and CD38 comprises a CD19 antigen binding component comprises a heavy chain immunoglobulin sequence set forth in SEQ ID NO: 301 or 304 and a light chain immunoglobulin sequence set forth in SEQ ID NO: 213, and the CD38 binding component comprises a heavy chain immunoglobulin sequence set forth in SEQ ID NO: 302, 303, 305-310 and a light chain immunoglobulin sequence set forth in SEQ ID NO: 213.

28. The method of any one of claims 1 to 26, wherein the bispecific antibody that binds CD19 and CD38 comprises an anti-CD19 immunoglobulin heavy chain variable region that comprises an A84S or an A108L substitution according to Kabat numbering.

29. The method of any one of claims 1 to 27, wherein the bispecific antibody that binds CD19 and CD38 comprises an anti-CD38 immunoglobulin light chain variable region comprises a W32H substitution according to Kabat numbering.

30. The method of any one of claims 1 to 28, wherein a single bispecific binding molecule is formed from the CD38 antigen binding component and the CD19 antigen binding component.

31. The method of any one of claims 1 to 30, wherein the bispecific antibody that binds CD19 and CD38 is a common light chain bispecific antibody.

32. The method of any one of claims 1 to 31, wherein the bispecific antibody that binds CD19 and CD38 is included in a formulation comprising a pharmaceutically acceptable diluent, carrier, or excipient.

33. The method of any one of claims 1 to 32, wherein the cancer or tumor is a solid-tissue cancer.

34. The method of claim 33, wherein the solid-tissue cancer comprises breast cancer, prostate cancer, pancreatic cancer, lung cancer, kidney cancer, stomach cancer, esophageal cancer, skin cancer, colorectal cancer, or head and neck cancer.

35. The method of claim 34, wherein the breast cancer is triple negative breast cancer, the lung cancer is non-small cell lung cancer, the head and neck cancer is head and neck squamous cell cancer, the kidney cancer is renal cell carcinoma, the brain cancer is glioblastoma multiforme, or the skin cancer is melanoma.

36. The method of any one of claims 1 to 32, wherein the cancer or tumor is a blood cancer

37. The method of claim 36, wherein the blood cancer is diffuse large B cell lymphoma.

38. The method of claim 36, wherein the blood cancer is myeloma.

39. The method of claim 36, wherein the blood cancer is Burkitt's lymphoma.

40. The method of claim 36, wherein the blood cancer is aggressive B cell lymphoma.

41. The method of claim 40, wherein the aggressive B cell lymphoma comprises double hit lymphoma, double expressor lymphoma, or triple hit lymphoma.

42. The method of any one of claims 37 to 41, wherein the blood cancer is relapsed or refractory.

43. The method of any one of claims 1 to 42, wherein the cancer or tumor associated with CD19 positive, CD38 high, immunosuppressive B cells is a cancer or tumor that comprises CD19 positive, CD38 high, B cell infiltrates.

44. The method of claim 43, wherein the CD19 positive, CD38 high immunosuppressive B cells express a B cell activation marker.

45. The method of claim 44, wherein the B cell activation marker comprises CD30.

46. The method of any one of claims 1 to 45, wherein the cancer or tumor associated with CD19 positive, CD38 high B cells expresses PD-L1.

47. The method of any one of claims 1 to 46, wherein the cancer or tumor associated with CD19 positive, CD38 high B cells is associated with CD20 low or CD20 negative B cells.

48. The method of claim 47, wherein the CD38 high B cells express at least about 30,000 CD38 proteins on the cell surface.

49. The method of claim 47, wherein the CD38 high B cells express at least about 35,000 CD38 proteins on the cell surface.

50. The method of claim 47, wherein the CD38 high B cells express at least about 40,000 CD38 proteins on the cell surface.

51. A method of treating an individual afflicted with a tumor or cancer, the method comprising performing an assay on the B cells of a biological sample of the individual for a CD38 high phenotype; and administering a bispecific antibody that binds CD19 and CD38 to the individual afflicted with the tumor or the cancer based on results of the assay on the B cells of a biological sample from the individual.

52. A method of treating an individual afflicted with a tumor or cancer, the method comprising administering a bispecific antibody that binds CD19 and CD38 to the individual afflicted with the tumor or the cancer based on results of an assay on B cells of a biological sample of the individual.

53. The method of claim 51 or 52, wherein the results of the assay of the B cells of the biological sample of the individual indicate a CD38 high phenotype.

54. The method of any one of claims 51 to 53, wherein the biological sample of the individual is a peripheral blood sample.

55. The method of any one of claims 51 to 53, wherein the biological sample of the individual is a tumor biopsy.

56. The method of any one of claims 51 to 55, wherein the assay the B cells of the individual comprises contacting the biological sample with an anti-CD38 antibody.

57. The method of any one of claims 51 to 56, wherein the assay comprises flow cytometry.

58. The method of any one of claims 51 to 56, wherein the assay comprises immunohistochemistry.

59. The method of any one of claims 51 to 58, wherein the individual is administered a bispecific antibody that binds CD38 and CD19 to the individual afflicted with the tumor or the cancer if greater than about 2% of the B cells of the individual exhibit a CD38 high phenotype.

60. The method of any one of claims 51 to 59, wherein the B cells of the biological sample of the individual indicate a CD38 high phenotype if the B cells express greater than about 30,000 cell surface CD38 molecules.

61. The method of any one of claims 51 to 59, wherein the B cells of the biological sample of the individual indicate a CD38 high phenotype if the B cells express greater than about 35,000 cell surface CD38 molecules.

62. The method of any one of claims 51 to 59, wherein the B cells of the biological sample of the individual indicate a CD38 high phenotype if the B cells express greater than about 40,000 cell surface CD38 molecules.

63. The method of any one of claims 51 to 62, wherein the bispecific antibody that binds CD19 and CD38 comprises a CD38 antigen binding component that comprises:
a) a heavy chain complementarity determining region 1 (HCDR1) comprising an amino acid sequence set forth in any one of SEQ ID NOs: 71-75;
b) a heavy chain complementarity determining region 2 (HCDR2) comprising an amino acid sequence set forth in any one of SEQ ID NOs: 81-85, or 151-155;
c) a heavy chain complementarity determining region 3 (HCDR3) comprising an amino acid sequence set forth in any one of SEQ ID NOs: 91-95;
d) a light chain complementarity determining region 1 (LCDR1) comprising an amino acid sequence set forth in any one of SEQ ID NOs: 101-105;
e) a light chain complementarity determining region 2 (LCDR2) comprising an amino acid sequence set forth in any one of SEQ ID NOs: 111-115; and
f) a light chain complementarity determining region 3 (LCDR3) comprising an amino acid sequence set forth in any one of SEQ ID NOs: 121-125; and wherein a CD19 antigen binding component comprises:
g) a heavy chain complementarity determining region 1 (HCDR1) comprising an amino acid sequence set forth in any one of SEQ ID NOs: 11-15;
h) a heavy chain complementarity determining region 2 (HCDR2) comprising an amino acid sequence set forth in any one of SEQ ID NOs: 21-25;
i) a heavy chain complementarity determining region 3 (HCDR3) comprising an amino acid sequence set forth in any one of SEQ ID NOs: 31-35;
j) a light chain complementarity determining region 1 (LCDR1) comprising an amino acid sequence set forth in any one of SEQ ID NOs: 101-105;
k) a light chain complementarity determining region 2 (LCDR2) comprising an amino acid sequence set forth in any one of SEQ ID NOs: 111-115; and
l) a light chain complementarity determining region 3 (LCDR3) comprising an amino acid sequence set forth in any one of SEQ ID NOs: 121-125.

64. The method of claim 63, wherein the CD38 antigen binding component comprises an HCDR2 amino acid sequence comprising the amino acid sequence set forth in SEQ ID NO: 151 to 155.

65. The method of claim 63, wherein the CD38 antigen binding component comprises an HCDR2 amino acid sequence comprising the amino acid sequence set forth in SEQ ID NO: 154.

66. The method of any one of claims 51 to 65, wherein the bispecific antibody that binds CD19 and CD38 comprises a CD38 antigen binding component comprises an HCDR2 amino acid sequence comprising any one of the amino acid sequences set forth in SEQ ID NO: 81 to 85.

67. The method of claims 51 to 66, wherein the bispecific antibody that binds CD19 and CD38 comprises an anti-CD38 immunoglobulin heavy chain variable region that comprises an amino acid sequence having at least about 90% identity to SEQ ID NO: 3 or 5; and the anti-CD38 immunoglobulin light chain variable region comprises an amino acid sequence having at least about 90% identity to SEQ ID NO: 4.

68. The method of claim 67, wherein the bispecific antibody that binds CD19 and CD38 comprises a anti-CD38 immunoglobulin heavy chain variable region that comprises an amino acid sequence identical to SEQ ID NO: 3 or 5; and the anti-CD38 immunoglobulin light chain variable region comprises an amino acid sequence identical to SEQ ID NO: 4.

69. The method of any one of claims 51 to 68, wherein the bispecific antibody that binds CD19 and CD38 comprises an anti-CD19 immunoglobulin heavy chain variable region that comprises an amino acid sequence having at least about 90% identity to SEQ ID NO: 1, 6; or 7 and the anti-CD19 immunoglobulin light chain variable region comprises an amino acid sequence having at least about 90% identity to SEQ ID NO: 2.

70. The method of claim 69, wherein the bispecific antibody that binds CD19 and CD38 comprises an anti-CD19 immunoglobulin heavy chain variable region that comprises an amino acid sequence identical to SEQ ID NO: 1, 6; or 7 and the anti-CD19 immunoglobulin light chain variable region comprises an amino acid sequence identical to SEQ ID NO: 2.

71. The method of any one of claims 51 to 70, wherein the bispecific antibody that binds CD19 and CD38 comprises a anti-CD38 immunoglobulin heavy chain variable region further comprises an immunoglobulin heavy chain constant region, wherein the anti-CD38 immunoglobulin heavy chain constant region comprises one or more amino acid substitutions that disfavors homodimerization of the anti-CD38 immunoglobulin heavy chain constant region and promotes heterodimerization of the anti-CD38 immunoglobulin heavy chain constant region with a non-anti-CD38 immunoglobulin heavy chain constant region.

72. The method of claim 71, wherein the bispecific antibody that binds CD19 and CD38 comprises an anti-CD38 immunoglobulin heavy chain constant region comprises a T366W substitution (EU numbering) or T366S/L368A/Y407V substitution (EU numbering), such that the heterodimerization of the anti-CD38 immunoglobulin heavy chain constant region and the non-anti-CD38 immunoglobulin heavy chain constant region is favored compared to homodimerization of the anti-CD38 immunoglobulin heavy chain.

73. The method of any one of claims 51 to 72, wherein the bispecific antibody comprises an anti-CD19 immunoglobulin heavy chain variable region further comprises an immunoglobulin heavy chain constant region, wherein the anti-CD19 immunoglobulin heavy chain constant region comprises one or more amino acid substitutions that disfavors homodimerization of the anti-CD19 immunoglobulin heavy chain constant region and promotes heterodimerization of the second heavy chain constant region with a non-anti-CD19 immunoglobulin heavy chain constant region.

74. The method of claim 73, wherein the anti-CD19 immunoglobulin heavy chain constant region comprises a T366W substitution (EU numbering) or a T366S/L368A/Y407V substitution (EU numbering), such that the heterodimerization of the anti-CD19 immunoglobulin heavy chain constant region and the non-anti-CD19 immunoglobulin heavy chain immunoglobulin heavy chain constant region is favored compared to homodimerization of the anti-CD19 immunoglobulin heavy chain.

75. The method of any one of claims 51 to 74, wherein the anti-CD38 immunoglobulin light chain variable region further comprises an immunoglobulin light chain constant region.

76. The method of any one of claims 51 to 75, wherein the bispecific antibody that binds CD19 and CD38 comprises a CD19 antigen binding component comprises a heavy chain immunoglobulin sequence set forth in SEQ ID NO: 301 or 304 and a light chain immunoglobulin sequence set forth in SEQ ID NO: 213, and the CD38 binding component comprises a heavy chain immunoglobulin sequence set forth in SEQ ID NO: 302, 303, 305-310 and a light chain immunoglobulin sequence set forth in SEQ ID NO: 213.

77. The method of any one of claims 51 to 76, wherein the bispecific antibody that binds CD19 and CD38 comprises an anti-CD19 immunoglobulin heavy chain variable region that comprises an A84S or an A108L substitution according to Kabat numbering.

78. The method of any one of claims 51 to 77, wherein the bispecific antibody that binds CD19 and CD38 comprises an anti-CD38 immunoglobulin light chain variable region comprises a W32H substitution according to Kabat numbering.

79. The method of any one of claims 51 to 78, wherein a single bispecific binding molecule is formed from the CD38 antigen binding component and the CD19 antigen binding component.

80. The method of any one of claims 51 to 79, wherein the bispecific antibody that binds CD19 and CD38 is a common light chain bispecific antibody.

81. The method of any one of claims 51 to 80, wherein the bispecific antibody that binds CD19 and CD38 is included in a formulation comprising a pharmaceutically acceptable diluent, carrier, or excipient.


SEQUENCE LISTING (311 sequences)

SEQ ID NO: 1 - protein, 123 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSS

SEQ ID NO: 2 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIK

SEQ ID NO: 3 - protein, 120 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSS

SEQ ID NO: 4 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 5 - protein, 120 aa, synthetic construct
QVQLVQSGAEVKKPGSSVKVSCKASGGTFSSYAFSWVRQAPGQGLEWMGRVIPFLGIANSAQKFQGRVTITADKSTSTAYMELSSLRSEDTAVYYCARDDIAALGPFDYWGQGTLVTVSS

SEQ ID NO: 6 - protein, 123 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRSEDTAVYYCARASGGSADYSYGMDVWGQGTLVTVSS

SEQ ID NO: 7 - protein, 123 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRSEDTAVYYCARASGGSADYSYGMDVWGGGTLVTVSS

SEQ ID NO: 8-10 - skipped (3 entries)

SEQ ID NO: 11 - protein, 8 aa, synthetic construct
GGTFSSYT

SEQ ID NO: 12 - protein, 5 aa, synthetic construct
SYTIN

SEQ ID NO: 13 - protein, 7 aa, synthetic construct
GGTFSSY

SEQ ID NO: 14 - protein, 6 aa, synthetic construct
SSYTIN

SEQ ID NO: 15 - protein, 10 aa, synthetic construct
GGTFSSYTIN

SEQ ID NO: 16-20 - skipped (5 entries)

SEQ ID NO: 21 - protein, 8 aa, synthetic construct
IIPIFGIP

SEQ ID NO: 22 - protein, 17 aa, synthetic construct
GIIPIFGIPNYAQKFQG

SEQ ID NO: 23 - protein, 4 aa, synthetic construct
PIFG

SEQ ID NO: 24 - protein, 13 aa, synthetic construct
WMGGIIPIFGIPN

SEQ ID NO: 25 - protein, 10 aa, synthetic construct
GIIPIFGIPN

SEQ ID NO: 26-30 - skipped (5 entries)

SEQ ID NO: 31 - protein, 16 aa, synthetic construct
ARASGGSADYSYGMDV

SEQ ID NO: 32 - protein, 14 aa, synthetic construct
ASGGSADYSYGMDV

SEQ ID NO: 33 - protein, 12 aa, synthetic construct
SGGSADYSYGMD

SEQ ID NO: 34 - protein, 15 aa, synthetic construct
ARASGGSADYSYGMD

SEQ ID NO: 35 - protein, 14 aa, synthetic construct
ASGGSADYSYGMDV

SEQ ID NO: 36-40 - skipped (5 entries)

SEQ ID NO: 41 - protein, 8 aa, synthetic construct
QGISSWLA

SEQ ID NO: 42 - protein, 11 aa, synthetic construct
RASQGISSWLA

SEQ ID NO: 43 - protein, 7 aa, synthetic construct
SQGISSW

SEQ ID NO: 44 - protein, 7 aa, synthetic construct
SSWLAWY

SEQ ID NO: 45 - protein, 11 aa, synthetic construct
RASQGISSWLA

SEQ ID NO: 46-51 - skipped (6 entries)

SEQ ID NO: 52 - protein, 7 aa, synthetic construct
AASSLQS

SEQ ID NO: 53 - skipped

SEQ ID NO: 54 - protein, 10 aa, synthetic construct
SLIYAASSLQ

SEQ ID NO: 55 - protein, 7 aa, synthetic construct
AASSLQS

SEQ ID NO: 56-60 - skipped (5 entries)

SEQ ID NO: 61 - protein, 9 aa, synthetic construct
QQYKRYPYT

SEQ ID NO: 62 - protein, 9 aa, synthetic construct
QQYKRYPYT

SEQ ID NO: 63 - protein, 6 aa, synthetic construct
YKRYPY

SEQ ID NO: 64 - protein, 8 aa, synthetic construct
QQYKRYPY

SEQ ID NO: 65 - protein, 9 aa, synthetic construct
QQYKRYPYT

SEQ ID NO: 66-70 - skipped (5 entries)

SEQ ID NO: 71 - protein, 8 aa, synthetic construct
GGTFSSYA

SEQ ID NO: 72 - protein, 5 aa, synthetic construct
SYAFS

SEQ ID NO: 73 - protein, 7 aa, synthetic construct
GGTFSSY

SEQ ID NO: 74 - protein, 6 aa, synthetic construct
SSYAFS

SEQ ID NO: 75 - protein, 10 aa, synthetic construct
GGTFSSYAFS

SEQ ID NO: 76-80 - skipped (5 entries)

SEQ ID NO: 81 - protein, 8 aa, synthetic construct
VIPFLGIA

SEQ ID NO: 82 - protein, 17 aa, synthetic construct
RVIPFLGIANSAQKFQG

SEQ ID NO: 83 - protein, 4 aa, synthetic construct
PFLG

SEQ ID NO: 84 - protein, 13 aa, synthetic construct
WMGRVIPFLGIAN

SEQ ID NO: 85 - protein, 10 aa, synthetic construct
RVIPFLGIAN

SEQ ID NO: 86-90 - skipped (5 entries)

SEQ ID NO: 91 - protein, 13 aa, synthetic construct
ARDDIAALGPFDY

SEQ ID NO: 92 - protein, 11 aa, synthetic construct
DDIAALGPFDY

SEQ ID NO: 93 - protein, 9 aa, synthetic construct
DIAALGPFD

SEQ ID NO: 94 - protein, 12 aa, synthetic construct
ARDDIAALGPFD

SEQ ID NO: 95 - protein, 11 aa, synthetic construct
DDIAALGPFDY

SEQ ID NO: 96-100 - skipped (5 entries)

SEQ ID NO: 101 - protein, 8 aa, synthetic construct
QGISSWLA

SEQ ID NO: 102 - protein, 11 aa, synthetic construct
RASQGISSWLA

SEQ ID NO: 103 - protein, 7 aa, synthetic construct
SQGISSW

SEQ ID NO: 104 - protein, 7 aa, synthetic construct
SSWLAWY

SEQ ID NO: 105 - protein, 11 aa, synthetic construct
RASQGISSWLA

SEQ ID NO: 106-111 - skipped (6 entries)

SEQ ID NO: 112 - protein, 7 aa, synthetic construct
AASSLQS

SEQ ID NO: 113 - skipped

SEQ ID NO: 114 - protein, 10 aa, synthetic construct
SLIYAASSLQ

SEQ ID NO: 115 - protein, 7 aa, synthetic construct
AASSLQS

SEQ ID NO: 116-120 - skipped (5 entries)

SEQ ID NO: 121 - protein, 9 aa, synthetic construct
QQYNSYPRT

SEQ ID NO: 122 - protein, 9 aa, synthetic construct
QQYNSYPRT

SEQ ID NO: 123 - protein, 6 aa, synthetic construct
YNSYPR

SEQ ID NO: 124 - protein, 8 aa, synthetic construct
QQYNSYPR

SEQ ID NO: 125 - protein, 9 aa, synthetic construct
QQYNSYPRT

SEQ ID NO: 126-149 - skipped (24 entries)

SEQ ID NO: 150 - protein, 6 aa, synthetic construct
  VARIANT 2: note = Any amino acid
  VARIANT 5: note = Any amino acid
PXLGXA

SEQ ID NO: 151 - protein, 6 aa, synthetic construct
PFLGTA

SEQ ID NO: 152 - protein, 6 aa, synthetic construct
PHLGIA

SEQ ID NO: 153 - protein, 6 aa, synthetic construct
PHLGTA

SEQ ID NO: 154 - protein, 6 aa, synthetic construct
PQLGIA

SEQ ID NO: 155 - protein, 6 aa, synthetic construct
PQLGTA

SEQ ID NO: 156 - protein, 6 aa, synthetic construct
  VARIANT 2: note = histidine, glutamine, threonine, asparagine, serine, glycine, alanine, arginine, lysine, aspartic acid, or glutamic acid
  VARIANT 5: note = histidine, glutamine, threonine, asparagine, serine, glycine, alanine, arginine, lysine, aspartic acid, or glutamic acid
PXLGXA

SEQ ID NO: 157-200 - skipped (44 entries)

SEQ ID NO: 201 - protein, 453 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 202 - protein, 450 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 479 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 204 - protein, 476 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIKGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 205 - protein, 476 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 473 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIKGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 687 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSQVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 208 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 209 - protein, 702 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGSQVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIK

SEQ ID NO: 210 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - skipped

SEQ ID NO: 212 - protein, 703 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIKGGGGGQVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 227 aa, synthetic construct; identical to SEQ ID NO: 208

SEQ ID NO: 215 - protein, 450 aa, synthetic construct
QVQLVQSGAEVKKPGSSVKVSCKASGGTFSSYAFSWVRQAPGQGLEWMGRVIPFLGIANSAQKFQGRVTITADKSTSTAYMELSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 453 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRSEDTAVYYCARASGGSADYSYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 217 - protein, 479 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRSEDTAVYYCARASGGSADYSYGMDVWGGGTLVTVSSGGGGSGGGGSGGGGSDIQMTQSPSSLSASVGDRVTITCRASQGISSWLAWYQQKPEKAPKSLIYAASSLQSGVPSRFSGSGSGTDFTLTISSLQPEDFATYYCQQYKRYPYTFGQGTKLEIKAAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 218 - protein, 120 aa, synthetic construct
QVQLVQSGAEVKKPGSSVKVSCKASGGTFSSYAFSWVRQAPGQGLEWMGRVIPFLGTANSAQKFQGRVTITADKSTSTAYMELSSLRSEDTAVYYCARDDIAALGPFDYWGQGTLVTVSS

SEQ ID NO: 219 - protein, 120 aa, synthetic construct
QVQLVQSGAEVKKPGSSVKVSCKASGGTFSSYAFSWVRQAPGQGLEWMGRVIPHLGIANSAQKFQGRVTITADKSTSTAYMELSSLRSEDTAVYYCARDDIAALGPFDYWGQGTLVTVSS

SEQ ID NO: 220 - protein, 120 aa, synthetic construct
QVQLVQSGAEVKKPGSSVKVSCKASGGTFSSYAFSWVRQAPGQGLEWMGRVIPHLGTANSAQKFQGRVTITADKSTSTAYMELSSLRSEDTAVYYCARDDIAALGPFDYWGQGTLVTVSS

SEQ ID NO: 221 - protein, 120 aa, synthetic construct
QVQLVQSGAEVKKPGSSVKVSCKASGGTFSSYAFSWVRQAPGQGLEWMGRVIPQLGIANSAQKFQGRVTITADKSTSTAYMELSSLRSEDTAVYYCARDDIAALGPFDYWGQGTLVTVSS

SEQ ID NO: 222 - protein, 120 aa, synthetic construct
QVQLVQSGAEVKKPGSSVKVSCKASGGTFSSYAFSWVRQAPGQGLEWMGRVIPQLGTANSAQKFQGRVTITADKSTSTAYMELSSLRSEDTAVYYCARDDIAALGPFDYWGQGTLVTVSS

SEQ ID NO: 223 - protein, 107 aa, synthetic construct
DIQMTQSPSSLSASVGDRVTITCRASQGISSHLAWYQQKPEKAPKSLIYAASSLQSGVPSRFSGSGSGTDFTLTISSLQPEDFATYYCQQYNSYPRTFGQGTKVEIK

SEQ ID NO: 224 - protein, 5 aa, synthetic construct
GSGGS

SEQ ID NO: 225 - protein, 5 aa, synthetic construct
GGGGS

SEQ ID NO: 226 - protein, 4 aa, synthetic construct
GGGS

SEQ ID NO: 227 - protein, 7 aa, synthetic construct
AAEPKSS

SEQ ID NO: 228 - protein, 17 aa, synthetic construct
AAEPKSSDKTHTCPPCP

SEQ ID NO: 229 - protein, 4 aa, synthetic construct
GGGG

SEQ ID NO: 230 - protein, 14 aa, synthetic construct
GGGGDKTHTCPPCP

SEQ ID NO: 231-300 - skipped (70 entries)

SEQ ID NO: 301 - protein, 453 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRAEDTAVYYCARASGGSADYSYGMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 302 - protein, 450 aa, synthetic construct
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 303 - protein, 450 aa, synthetic construct
QVQLVQSGAEVKKPGSSVKVSCKASGGTFSSYAFSWVRQAPGQGLEWMGRVIPFLGIANSAQKFQGRVTITADKSTSTAYMELSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 304 - protein, 453 aa, synthetic construct
QVQLVQSGAEVKKPGSSVKVSCKASGGTFSSYTINWVRQAPGQGLEWMGGIIPIFGIPNYAQKFQGRVTITADESTNTAYMELSSLRSEDTAVYYCARASGGSADYSYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 305 - protein, 450 aa, synthetic construct
QVQLVQSGAEVKKPGSSVKVSCKASGGTFSSYAFSWVRQAPGQGLEWMGRVIPFLGTANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 450 aa, synthetic construct
QVQLVQSGAEVKKPGSSVKVSCKASGGTFSSYAFSWVRQAPGQGLEWMGRVIPHLGIANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 450 aa, synthetic construct
QVQLVQSGAEVKKPGSSVKVSCKASGGTFSSYAFSWVRQAPGQGLEWMGRVIPQLGIANSAQKFQGRVTITADKSTSTAYMDLSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 450 aa, synthetic construct
QVQLVQSGAEVKKPGSSVKVSCKASGGTFSSYAFSWVRQAPGQGLEWMGRVIPFLGTANSAQKFQGRVTITADKSTSTAYMELSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 309 - protein, 450 aa, synthetic construct
QVQLVQSGAEVKKPGSSVKVSCKASGGTFSSYAFSWVRQAPGQGLEWMGRVIPHLGIANSAQKFQGRVTITADKSTSTAYMELSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 310 - protein, 450 aa, synthetic construct
QVQLVQSGAEVKKPGSSVKVSCKASGGTFSSYAFSWVRQAPGQGLEWMGRVIPQLGIANSAQKFQGRVTITADKSTSTAYMELSSLRSEDTAVYYCARDDIAALGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 311 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK